(19) United States
(12) Patent Application Publication
(10) Pub. No.: US 2006/0002921 A1
(43) Pub. Date: Jan. 5, 2006

(54) Optimized dosing with anti-CD4 antibodies for tolerance induction in primates

(75) Inventors: Dawn Winsor-Hines, Framingham, MA (US); Patricia Rao, Acton, MA (US); Douglas J. Ringler V, Boston, MA (US); Paul Ponath, San Francisco, CA (US)

Correspondence Address: LAHIVE & COCKFIELD, LLP., 28 STATE STREET, BOSTON, MA, 02109, US

(73) Assignee: TolerRX, Inc., Cambridge, MA (US)

(21) Appl. No.: 11/158,505

(22) Filed: Jun. 21, 2005

Related U.S. Application Data
(60) Provisional application No. 60/582,181, filed on Jun. 22, 2004.

(51) Int. Cl. A61K 39/395

(52) U.S. Cl. 424/141.1; 424/144.1

(57) ABSTRACT

The present invention is based, at least in part, on the finding that tolerance can be induced by inhibition of CD4+ cells (and optionally CD8+ cells). Accordingly, the optimized dosing methods of the invention are useful in treating a primate, e.g., a human, by inhibiting CD4+ T cells to induce tolerance to at least one antigen, e.g., self or foreign, such as for inducting tolerance in a primate against a soluble or a cell bound antigen (e.g., an allogeneic or xenogeneic transplanted antigen).

### RELATED APPLICATIONS

This application claims the benefit of U.S. Provisional Application 60/582,181, filed Jun. 22, 2004, titled “Optimized Dosing of Anti-CD4 Antibodies for Tolerance Inducing Induction in Primates”. This application is related to U.S. Provisional Application 60/431,839, filed Dec. 9, 2002, titled “Introducing Tolerance to Proteins in Primates,” and U.S. Ser. No. 10/731,984 “Introducing Tolerance in Primates filed Dec. 9, 2003. The entire contents of each of these applications are incorporated herein by reference.

### BACKGROUND OF THE INVENTION

This invention relates to tolerance induction and more particularly to inducing tolerance in a primate against an antigen(s) and in particular a foreign antigen.

There have been numerous attempts to induce tolerance against foreign and self antigens in primates. For example, in the field of transplantation, there is a need to induce tolerance to foreign antigens in a transplant so as to prevent its rejection. At present, rejection can only be prevented by the use of long-term (chronic) immunosuppression which carries risks of infection, cancer and drug toxicity.

In addition, in the treatment of a patient with a therapeutic protein, in many cases, treatment becomes less effective or totally ineffective as a result of an immune response to that foreign protein.

Although success has been demonstrated using anti-CD4 antibodies in rodents, tolerance induction with anti-CD4 antibodies has yet to be demonstrated in primates. As a result, there is a need for a treatment that induces tolerance to antigen(s), e.g., cell bound or soluble proteins that reduces or eliminates the need for immunosuppressive drugs and long term immune suppression in a primate against an antigen(s) and in particular a foreign protein(s).

### SUMMARY OF THE INVENTION

The present invention advances the art by providing optimized doses of anti-CD4 that are able to reduce immune responses to foreign proteins without long term immunosuppression. In accordance with an aspect of the present invention, there is provided a process for tolerizing a primate against an antigen(s) by use of a CD4 antibody or fragment thereof in an amount and for a time effective to induce tolerance against at least one antigen. In one embodiment, the anti-CD4 antibody is administered in combination with a second agent that promotes tolerance. In one embodiment, the second agent is an anti-CD8 antibody or other agent that inhibits the activity of CD8+ cells.

In one aspect, the invention is directed to a method for treating a primate to induce tolerance to at least one foreign antigen comprising, administering to the primate at least one anti-CD4 antibody or CD4 binding fragment thereof, wherein the at least one anti-CD4 antibody is administered at a dose of between about 20 mg/kg to 40 mg/kg in at least three separate doses.

In one embodiment, the at least one anti-CD4 antibody is administered at a dose of about 20 mg/kg.

In another embodiment, the at least one anti-CD4 antibody is administered in at least four separate doses. In another embodiment, the at least one anti-CD4 antibody is administered in at least five separate doses.

In one embodiment, the at least one anti-CD4 antibody is administered on at least days −1, 3 or 4, 8 and 12 relative to administration of the foreign antigen.

In one embodiment, wherein the at least one anti-CD4 antibody is administered on at least days −1, 1, and 3 relative to administration of the foreign antigen.

In one embodiment, the foreign antigen is a soluble antigen.

In one embodiment, the first dose of the at least one anti-CD4 antibody is administered prior to administration of the foreign antigen.

In one embodiment, the at least one anti-CD4 antibody is TRX1.

In another aspect, the invention is directed to a method for treating a primate to induce tolerance to at least one foreign antigen comprising, administering to the primate at least one anti-CD4 antibody or CD4 binding fragment thereof, wherein at least one dose of the at least one anti-CD4 antibody is administered at least one day prior to administration of the foreign antigen.

In yet another aspect, the invention is directed to a method for treating a primate to induce tolerance to at least one foreign antigen comprising, administering to the primate at least one anti-CD4 antibody or CD4 binding fragment thereof, wherein the at least one anti-CD4 antibody is administered at a dose sufficient to maintain a serum concentration of anti-CD4 antibody at a level of about 20 μg/ml during the tolerance induction phase.

In one embodiment, at least one dose of the at least one anti-CD4 antibody is administered one day prior to administration of the foreign antigen.

In another embodiment, the at least one anti-CD4 antibody is administered at a dose of between about 20 mg/kg and 40 mg/kg.

In another aspect, the invention is directed to a method for treating a primate to induce tolerance to at least one foreign antigen comprising, administering to the primate at least one anti-CD4 antibody or CD4 binding fragment thereof, wherein the at least one anti-CD4 antibody is administered at a dose sufficient to achieve about 85% saturation of CD4 sites on T cells in the primate during the tolerance induction phase.

In one embodiment, the at least one anti-CD4 antibody is not administered for more than about two weeks.

In one embodiment, at least one dose of the at least one anti-CD4 antibody is administered one day prior to administration of the foreign antigen.

In another embodiement, the at least one anti-CD4 antibody is administered at a dose of between about 20 mg/kg and 40 mg/kg.

In one embodiment, the at least one anti-CD4 antibody is TRX1.

In another aspect, the invention pertains to a method for inducing tolerance in a primate to a soluble antigen, comprising, administering to a primate at least one anti-CD4 antibody or CD4 binding fragment thereof, wherein the at least one anti-CD4 antibody is administered at a dose of between about 20-40 mg/kg in at least three separate doses, such that tolerance to the soluble antigen is induced.

In one embodiment, at least one dose of the at least one anti-CD4 antibody is administered one day prior to administration of the soluble antigen.

In another embodiment, the at least one anti-CD4 antibody is administered at a dose of about 20 mg/kg.

### Brief Description of the Drawings

FIG. 1A shows the amino acid sequence of the first embodiment of TRX1 antibody light chain. FIG. 1B shows the nucleotide sequence of the first embodiment of TRX1 antibody light chain. FIG. 1C shows the amino acid sequence of the first embodiment of TRX1 antibody light chain with and without a leader sequence. FIG. 1D shows the amino acid sequence of the first embodiment of TRX1 antibody heavy chain. FIG. 1E is a continuation of the sequence shown in FIG. 1D. FIG. 1F shows the nucleotide sequence of the first embodiment of TRX1 antibody heavy chain. FIG. 1G shows the amino acid sequence of the first embodiment of TRX1 antibody heavy chain with and without a leader sequence.

FIG. 2A shows the amino acid sequence of another embodiment of TRX1 antibody light chain. FIG. 2B shows the nucleotide sequence of another embodiment of TRX1 antibody light chain. FIG. 2C shows the amino acid sequence of another embodiment of TRX1 antibody light chain with and without a leader sequence. FIG. 2D shows the amino acid sequence of another embodiment of TRX1 antibody heavy chain. FIG. 2E is a continuation of the sequence shown in FIG. 2D. FIG. 2F shows the nucleotide sequence of another embodiment of TRX1 antibody heavy chain. FIG. 2G shows the amino acid sequence of another embodiment of TRX1 antibody heavy chain with and without a leader sequence.

FIG. 3A shows the amino acid sequence of another embodiment of TRX1 antibody light chain. FIG. 3B shows the nucleotide sequence of another embodiment of TRX1 antibody light chain. FIG. 3C shows the amino acid sequence of another embodiment of TRX1 antibody light chain with and without a leader sequence. FIG. 3D shows the amino acid sequence of another embodiment of TRX1 antibody heavy chain. FIG. 3E is a continuation of the sequence shown in FIG. 3D. FIG. 3F shows the nucleotide sequence of another embodiment of TRX1 antibody heavy chain. FIG. 3G shows the amino acid sequence of another embodiment of TRX1 antibody heavy chain with and without a leader sequence.

FIG. 4A shows the amino acid sequence of another embodiment of TRX1 antibody light chain. FIG. 4B shows the nucleotide sequence of another embodiment of TRX1 antibody light chain. FIG. 4C shows the amino acid sequence of another embodiment of TRX1 antibody light chain with and without a leader sequence. FIG. 4D shows the amino acid sequence of another embodiment of TRX1 antibody heavy chain. FIG. 4E is a continuation of the sequence shown in FIG. 4D. FIG. 4F shows the nucleotide sequence of another embodiment of TRX1 antibody heavy chain. FIG. 4G shows the amino acid sequence of another embodiment of TRX1 antibody heavy chain with and without a leader sequence.

FIGS. 5A-5C show the sequence of the heavy chains of the humanized CD8 antibody used in Example 5.

FIG. 6 shows the sequence of the light chains of the humanized CD8 antibody used in Example 5.

FIGS. 7A-7C show another embodiment of a TRX1 heavy chain. In this embodiment, the amino acid at position 236 is a Leu, the amino acid at position 238 is a Gly, and the amino acid at position 297 is an Ala.

FIGS. 8A-8B show another embodiment of a TRX1 light chain. In this embodiment, the amino acid at position 117 is a Leu.

FIGS. 9A-9B show a schematic overview of the tolerance induction and antigen challenge protocol. 9A, The protocol was divided into 3 phases: induction, washout, and challenge. During the induction phase TRX1 antibody or saline was infused on days −1, 3 or 4, 8 and 12. Antigen (equine Ig or saline) was administered on days 0, 3 and 8. The induction phase was followed by a washout phase during which serum levels of TRX1 and equine Ig were monitored until no longer detectable. The challenge phase was initiated on day 68 by dosing all animals with equine Ig as well as a neoantigen, SRBC. Additional equine Ig challenges were administered on day 95 and day 130. 9B, Treatment groups consisted of 4 experimental TRX1 dosing cohorts and 2 control groups. The experimental groups received 4 infusions of TRX1 at 1, 10, 20, or 40 mg/kg and 3 doses of antigen. Control group I, antigen only, received 4 infusions of saline and 3 doses of equine Ig. Control group II, TRX1 only, was comprised of 2 cohorts with animals receiving 4 infusions of TRX1 at 20 or 40 mg/kg. Animals in control group II received 3 doses of saline rather than antigen. All animals were challenged three times with antigen and received a single immunization with SRBC at the time of the first equine Ig challenge.

FIGS. 10A-10C show pharmacokinetics and pharmacodynamics of TRX1 during the induction and washout phases. 10A, Group mean TRX1 serum concentrations (μg/ml). Experimental and control group II animals receiving equivalent TRX1 doses (20 and 40 mg/kg) are combined. The arrow indicates dosing with TRX1. Open symbols represent animals grouped according to the dose of TRX1 received, 1 mg/kg (n=3); 10 mg/kg (n=3); 20 mg/kg (n=4); or 40 mg/kg (n=6); 10B, Saturation of CD4 sites on CD3+ cells in peripheral blood as a function of TRX1 dose during tolerance induction and washout phases. TRX1-biotin staining of whole blood samples was used to detect free CD4 sites. The mean MCF value for each group is represented as a percent of the group mean MCF baseline value; Control group I, antigen only, closed circles (n=3); open symbols represent animals grouped according to the dose of TRX1 received, 1 mg/kg group (n=3); 10 mg/kg (n=3); 20 mg/kg; and 40 mg/kg (n=6); 10C, Total CD4⁺ T cells per ml of blood. Group mean absolute CD4⁺ lymphocyte counts are calculated as a percentage of group mean baseline values; Control group I, antigen only, closed circles (n=3); open symbols represent animals grouped according to the dose of TRX1 received, 1 mg/kg (n=3); 10 mg/kg (n=3); 20 mg/kg (n=4); 40 mg/kg (n=6).

FIGS. 11A-11C show immune response during induction and first challenge. 11A, Group mean antibody titers generated against equine Ig during the induction phase. Animals received 3 doses of antivenin indicated by arrows. Titer against antivenin is defined as the reciprocal of the dilution resulting in an OD value equivalent to twice the OD value of a 1:25,000 dilution of a positive control standard. Closed circles are control group I, antigen only (n=3); open symbols represent the TRX1 experimental dosing cohorts, 1 mg/kg TRX1 (n=3); 10 mg/kg TRX1 (n=3); 20 mg/kg TRX1 (n=2); and 40 mg/kg TRX1 (n=3); 11B, Group mean antibody titers generated against equine Ig after the first challenge given on day 68 (arrow). Closed circles are control group I, antigen only (n=3); gray symbols are control group II, TRX1 only cohorts, 20 mg/kg TRX1 (n=2); and 40 mg/kg (n=3); and open symbols represent the TRX1 experimental dosing cohorts, 1 mg/kg (n=3); 10 mg/kg (n=3); 20 mg/kg (n=3); and 40 mg/kg (n=3); 11C, Immune response to the neo-antigen, SRBC, administered at the time of first challenge on day 68 (arrow) and measured by hemolysis of SRBC. Group mean antibody titers for control group I, closed circles, (n=3); control group II cohorts, gray symbols, 20 mg/kg TRX1 (n=2); and 40 mg/kg (n=3); and TRX1 experimental dosing cohorts, open symbols, 1 mg/kg (n=3); 10 mg/kg (n=3); 20 mg/kg (n=3); and 40 mg/kg (n=3). Titer is defined as the reciprocal of the highest dilution of serum that did not cause obvious hemolysis.

FIGS. 12A-12B show immune response to equine Ig after multiple challenges. 12A, Group mean antibody titers for control group I, closed circles, (n=3); Control group II cohorts, gray symbols, 20 mg/kg TRX1 (n=2) and 40 mg/kg (n=3); and the TRX1 experimental group cohorts, open symbols, 20 mg/kg TRX1 (n=2) and 40 mg/kg TRX1 (n=3). 12B, Antibody titers to equine Ig of individual animals in the TRX1 experimental group 20 mg/kg (#16276 and #16096, open symbols, solid lines) and 40 mg/kg (#16178, #16192, and #16286, closed symbols, solid lines) cohorts plotted with the group mean antibody titers to equine Ig for control group I (gray circles, solid line) and control group II, 20 mg/kg (open triangle, dotted line, n=2) and 40 mg/kg (closed square, dotted line, n=3) cohorts.

FIGS. 13A-13B show immune response to equine Ig with modified TRX1 dosing. Closed circles represent saline control group I, or antigen only (n=3); open triangles represent the 20 mg/kg TRX1 treated experimental group (n=2). Titer is defined as the reciprocal of the dilution of serum resulting in an OD value equivalent to twice the OD value of a 1:25,000 dilution of positive control serum. 13A, Group mean antibody titers generated against equine Ig during the induction phase. Animals received 3 doses of TRX1 indicated by arrows on day −1, 1 and 3. Equine Ig was administered on days 0, 4 and 8. 13B, Group mean antibody titers generated against equine Ig during the challenge phase. Animals were challenged with 10 mg/kg antivenin s.c. on days 68 and 97 and with 1 mg/kg equine Ig s.c. on day 133.

### DETAILED DESCRIPTION OF THE INVENTION

The antigen(s) as to which tolerance is induced may be a self antigen or a foreign antigen and in particular a foreign antigen(s).

As used herein, the term “tolerize” or “tolerant” or “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2 upon subsequent exposure to the tolerizing antigen. Tolerance can occur to self antigens or to foreign antigens. In one embodiment, the a tolerant primate does not produce an adverse immune response to the antigen over a period of time after treatment with a tolerizing agent is stopped even when subsequently challenged with the antigen and/or when the antigen remains present in the primate, but is capable of providing an immune response against other antigens. In one embodiment, tolerance is induced in the absence of a therapeutic level of a general immunosuppressant.

For example, the foreign antigen may be one or more of the following types of antigens:
(i) a foreign antigen(s) present on transplanted tissue or cells, including tissue or cells present in an organ wherein the transplant may be allogeneic or xenogeneic;
(ii) a therapeutic agent (which also includes therapeutic agents used for disease prevention) that produces an immune response in a primate, which immune response diminishes the ability of the agent to function as a therapeutic agent. Such agents include, but are not limited to, delivery vehicles, such as vectors used in gene therapy; active agents such as proteins delivered to the primate (e.g., recombinant proteins such as monoclonal antibodies, enzymes, clotting factors) and some small molecule drugs or proteins produced from an agent delivered to the primate, such as in gene therapy.

The foreign antigens against which tolerance is induced in accordance with the present invention are not foreign antigens as present in disease-causing bacteria, fungi, viruses, etc. that infect a host, i.e., the term foreign antigen does not include a foreign antigen as part of an organism that infects a primate and causes a disease or disorder.

In one embodiment, the antigen is a soluble antigen.

The CD4 antibody or CD8 antibody in the case where a CD8 antibody is used, is preferably a monoclonal antibody (or fragment thereof that retains the ability to bind to CD4 or CD8, respectively). The antibody may be a human antibody or a non-human antibody, with non-human antibodies including humanized antibodies, chimeric antibodies, murine antibodies, etc.

The CD4 antibody or appropriate fragment thereof is administered to a primate in an amount and for a time effective to induce tolerance against a foreign or self antigen and preferably a foreign antigen. Anti-primate CD4 antibodies are known in the art as are methods of making such antibodies.

In one embodiment, the anti-CD4 antibody is administered prior to exposure (or systemic exposure) of the subject to the antigen to which tolerance is desired. In another embodiment, the anti-CD4 antibody is administered simultaneously with the antigen to which tolerance is desired.

In certain embodiments, in particular where tolerance to a transplant (e.g., a cell or tissue transplant) is desired, it may be desirable to additionally inhibit CD8+ cells. The compound that inhibits CD8+ T cells inhibits the activity of CD8+ T cells, e.g., by reducing their number or by inhibiting their effector function. In one embodiment, a compound that inhibits CD8+ T cells specifically inhibits CD8+ T cells. In another embodiment, a compound that inhibits CD8+ T cells does not significantly inhibit or deplete Treg cells. Such a compound may be an antibody that does or does not deplete CD8+ T cells. Anti-primate CD8 antibodies are known in the art as are methods for making such antibodies. The compound that inhibits CD8+ T-cells may be a compound (other than an antibody) that inhibits such CD8+ T cells (such compound other than an antibody may or may not deplete CD8+ T cells. Exemplary non-antibody compounds include, e.g., beta-galactoside-binding protein (Blaser et al. 1998. Eur J. Immunol. 28:2311).

In one embodiment, the compound that inhibits CD8+ T cells is administered prior to administration of the anti-CD4 antibody. In another embodiment, the compound that inhibits CD8+ T cells is administered simultaneously with the anti-CD4 antibody. In another embodiment, the compound that inhibits CD8+ T cells is administered subsequent to administration of the anti-CD4 antibody.

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4+ CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

Representative examples of compounds (other than antibodies) that inhibit CD8+ T cells include: Rapamycin (sirolimus) and CellCept® (mycophenolate mofetil). In one embodiment, a compound such as cyclosporin is preferably not used because, although it inhibits CD8+ T cells, such compound also inhibits Treg cells (e.g., by depletion).

The present invention has particular applicability to inducing tolerance in a primate with respect to a transplant. Preferably such a primate is a human. The transplant may be allogeneic or xenogeneic.

In accordance with a preferred embodiment, each of the CD4 antibody or appropriate fragment thereof and the CD8 inhibiting compound is administered over a period of time in order to maintain in the primate appropriate levels of such antibody or fragment and compound over a period of time that is sufficient to induce tolerance.

In one embodiment, at least one anti-CD4 antibody or CD4 binding fragment thereof, wherein the at least one anti-CD4 antibody is administered at a dose of about 20 mg/kg. In another embodiment, the at least one anti-CD4 antibody is administered at a dose of between about 20 and 40 mg/kg. In another embodiment, the at least one anti-CD4 antibody is administered at a dose of at least about 20 mg/kg. In one embodiment, at least three doses of the antibody are administered. Each dose may be administered over time, e.g., may be diluted and infused over a twenty four hour period, or portions of the dose may be administered over the course of a twenty four hour period, e.g., in separate inoculations.

In another embodiment, four separate doses of the anti-CD4 antibody are administered, e.g., the at least one anti-CD4 antibody is administered on at least four separate days.

In another embodiment, five separate doses of the anti-CD4 antibody are administered, e.g., the at least one anti-CD4 antibody is administered on at least five separate days.

In another embodiment, at least one dose of the at least one anti-CD4 antibody is administered at least about 7 days prior to administration of the foreign antigen. In another embodiment, at least one dose of the at least one anti-CD4 antibody is administered at least about 5 days prior to administration of the foreign antigen. In another embodiment, at least one dose of the at least one anti-CD4 antibody is administered at least about 4 days prior to administration of the foreign antigen. In another embodiment, at least one dose of the at least one anti-CD4 antibody is administered at least about 3 days prior to administration of the foreign antigen. In another embodiment, at least one dose of the at least one anti-CD4 antibody is administered at least about 2 days prior to administration of the foreign antigen. In another embodiment, at least one dose of the at least one anti-CD4 antibody is administered at least about 1 day prior to administration of the foreign antigen.

In one embodiment, administration of at least one anti-CD4 antibody continues for approximately one week after exposure to the foreign antigen. In yet another embodiment, administration of at least one anti-CD4 antibody continues for approximately two weeks after exposure to the foreign antigen. In another embodiment, administration of at least one anti-CD4 antibody continues for approximately one month after exposure to the foreign antigen.

In another embodiment, the at least one anti-CD4 antibody is administered on at least days −1, 3 or 4, 8 and 12 relative to administration of the foreign antigen.

In yet another embodiment, the at least one anti-CD4 antibody is administered on at least days −1, 1, and 3 relative to administration of the foreign antigen.

In another embodiment, the invention pertains to a process for treating a primate to induce tolerance to at least one foreign antigen comprising, administering to the primate at least one anti-CD4 antibody or CD4 binding fragment thereof, wherein the at least one anti-CD4 antibody is administered at a dose sufficient to achieve slightly less than complete saturation of CD4 sites, e.g., about 95%, about 90%, about 85%, about 80%, or about 75% saturation of CD4 sites on T cells in the primate during the tolerance induction phase. In another embodiment, at least one anti-CD4 antibody is administered at a dose sufficient to achieve slightly less than complete saturation of CD4 sites, e.g., about 95%, about 90%, about 85%, about 80%, or about 75% saturation of CD4 sites on T cells in the primate during the tolerance induction phase. In one embodiment, the level of saturation of CD4 sites does not exceed about 85%. CD4 saturation can be determined using methods known in the art. For example, in the appended examples, saturation was determined as a function of free CD4 sites on circulating lymphocytes. For example, free CD4 sites can be determined by staining with anti-CD4, e.g., comprising a detectable label.

In one embodiment, administration of anti-CD4 antibody is not necessary after the tolerance induction phase.

In still another embodiment, the invention pertains to a process for inducing tolerance in a primate to a transplanted antigen, comprising, administering to a primate at least one anti-CD4 antibody or CD4 binding fragment thereof and at least one compound that inhibits CD8+ T cells each in an amount and for a time effective to induce tolerance against the transplant, said anti-CD4 antibody or fragment being present in said primate when said transplanted antigen is present in said primate and said anti-CD4 antibody being administered in an initial dose of about 20 mg/kg, such that tolerance to the transplanted antigen is induced.

In one embodiment, the dose of anti-CD4 may vary at each of the times given during the induction phase. For example, the CD4 antibody (or fragment thereof) may be administered in an initial dose, e.g. as set forth herein, and subsequent doses may be greater than, equal to, or less than the initial dose.

Preferably, the dose of anti-CD4 administered (either the initial or subsequent doses) is at least about 20 mg/kg. In another embodiment, less than or equal to 40 mg/kg is administered in a dose. In another embodiment, between about 20 mg/kg and 40 mg/kg is administered in a dose. In yet another embodiment, between about 10 mg/kg and 40 mg/kg is administered in a dose. In yet another embodiment, between about 5 and 40 mg/kg is administered in a dose.

In one embodiment, the CD4 antibody (or fragment thereof) may be administered in a dose of less than or equal to about 20 mg/kg. In another embodiment, the CD4 antibody (or fragment thereof) may be administered in a dose of less than about 20 mg/kg. For example, in instances when the anti-CD4 antibody is administered to humans, it may be desirable to reduce the amount of anti-CD4 antibody administered given the reduced immunogenicity and improved pharmacokinetics of the TRX1 antibody in humans. For example, in one embodiment, the dose of anti-CD4 administered (either the initial or subsequent doses) is at least about 5 mg/kg. In another embodiment, the dose of anti-CD4 administered (either the initial or subsequent doses) is at least about 7.5 mg/kg. In another embodiment, the dose of anti-CD4 administered (either the initial or subsequent doses) is at least about 10 mg/kg. In another embodiment, the dose of anti-CD4 administered (either the initial or subsequent doses) is at least about 12.5 mg/kg. In another embodiment, the dose of anti-CD4 administered (either the initial or subsequent doses) is at least about 15 mg/kg. In another embodiment, the dose of anti-CD4 administered (either the initial or subsequent doses) is at least about 17.5 mg/kg.

The initial dose of the CD4 antibody may be administered in one or more parts over a twenty-four hour period and preferably in one dose over twenty-four hours.

As used herein in reference to a dosage amount, a dose is the total amount of the CD4 antibody administered over a twenty-four hour period, even if administered more than once in 24 hours.

As used herein, the term “tolerance induction phase” includes the time during which anti-CD4 antibody is administered to a primate to induce tolerance. For example, anti-CD4 antibody may be administered in three to four doses over a short time period (e.g., for about a month or less, such as about 10, about 13, about 15, about 20, about 25, or about 30 days) in close proximity to the time of exposure to the foreign antigen.

In most cases, after the initial dose, the CD4 antibody (or appropriate fragment thereof) is administered in one or more follow-up doses over several day(s), with each follow-up dose being administered in one or more doses in a twenty-four hour period. The follow-up dose(s) is generally provided in an amount to return serum levels of the CD4 antibody to those that were achieved by the initial dose.

In a preferred embodiment, the minimum follow-up dose or doses of the CD4 antibody is in an amount that is generally equal to the amounts hereinabove described and may or may not be identical to the dose given as the original or initial dose.

If there is more than one follow-up dose of the CD4 antibody, each such additional follow-up dose over a 24-hour period may be the same or different than another follow-up dose.

The number of follow-up doses of the CD4 antibody will vary. In one embodiment, there is at least one follow-up dose. In one embodiment, the total number of doses does not exceed eight daily doses.

In one embodiment, subsequent doses of anti-CD4 antibody are administered approximately every 4-5 days. In another embodiment, subsequent doses of anti-CD4 antibody are administered approximately every 2-3 days. In another embodiment, subsequent doses of anti-CD4 antibody are administered approximately every 1-2 days.

In one embodiment, the total period over which the CD4 antibody is administered does not exceed four weeks and more preferably does not exceed three weeks. In many cases, tolerance can be achieved by using an initial dose and one or more follow-up doses over a period that does not exceed two weeks.

Although, in accordance with the present invention, initial tolerance to an antigen(s) can be achieved in a primate in a tolerance induction period of no more than about four weeks, in some cases, periodic follow-up treatments with the CD4 antibody may be administered in order to maintain tolerance.

In one embodiment, the invention pertains to a process for treating a primate to induce tolerance to at least one foreign antigen comprising, administering to the primate at least one anti-CD4 antibody or CD4 binding fragment thereof, wherein the at least one anti-CD4 antibody is administered at a dose sufficient to maintain a serum concentration of anti-CD4 antibody at a level of about 20 μg/ml during the tolerance induction. In another embodiment, the serum concentration is maintained at a level of at least about 20 μg/ml.

As hereinabove described, at least one CD4 antibody (or appropriate fragment thereof) is delivered in an amount that is at least sufficient to induce tolerance in a primate against an antigen(s) and in a preferred embodiment against a foreign antigen. The maximum amount is of course limited by safety considerations. In general, the daily dosage of CD4 antibody would be less than 6000 mg.

The number of follow-up doses and the spacing thereof will be determined, in part, by the half life of the at least one CD4 antibody. Although the present invention is not to be limited, in one embodiment, the CD4 antibody will be initially delivered in an amount to achieve antibody serum levels that exceed the amount required to saturate all of the CD4 of the primate being treated, with follow-up doses being given at times to maintain such excess over a period that induces tolerance in the primate against the foreign antigen(s).

In a preferred embodiment, the CD4 antibody is a CD4 antibody that would have a reduced effector (i.e. lytic) function as compared to human IgG1. Examples of antibodies that would have reduced effector function, include antibodies that have an Fc portion that is aglycosylated and/or that has reduced binding to the Fc receptor and/or is non-lytic. For example, in one embodiment, an anti-CD4 antibody comprises at least one mutation in the constant region of the heavy chain. Exemplary mutations include Leu 236 to Ala (e.g., CTG to GCG), Gly 238 to Ala (e.g., GGA to GCA), Asn 297 to Ala (e.g., AAC to GCC). In one embodiment, one or more of these mutations may be made. For example, in a preferred embodiment, the mutation at position 297 is made to produce an aglycosyl anti-CD4 antibody with reduced effector function. In another embodiment, the mutations at positions 236 and 238 are made. This form is glycosylated, but Fc receptor and complement binding are reduced.

In one embodiment, a CD4 antibody with a reduced effector function is a non-depleting CD4 antibody. As used herein, “a non-depleting CD4 antibody” is a CD4 antibody that depletes less than 50% of CD4 cells and preferably less than 10% of CD4 cells.

In one embodiment, a cocktail comprising different anti-CD4 antibodies can be used. In another embodiment, different anti-CD4 antibodies can be administered to the same patient on different days.

In one embodiment, a CD8 cell inhibiting compound is further administered to enhance tolerance in a primate. The CD8 inhibiting compound is administered to the primate during the initial treatment with the CD4 antibody in an amount effective to reduce the action and/or level of CD8+ T cells in the primate. Such amounts may be lower than the amounts used for the CD4 antibody. The CD8 inhibiting compound may be used at the same time as the CD4 antibody or may be used at different times. The CD8 inhibiting compound may be administered on different days or on the same day as the CD4 antibody. As hereinabove described, the CD8 inhibiting compound may be an antibody (or fragment thereof) or a compound other than an antibody. The treatment with the CD8 inhibiting compound is performed during the initial treatment (including initial follow-up doses); however, if further treatment with CD4 antibody is used after the initial treatment period (including follow-up doses), such further treatment may be performed with or without treatment with the CD8 inhibiting compound.

In treating a primate and in particular a human, the CD4 antibody (and optionally the CD8 inhibiting compound) may be employed in combination with a pharmaceutically acceptable carrier, e.g., formulated for separate or joint administration. A composition that contains a CD4 antibody and/or CD8 inhibiting compound may include other ingredients, for example, stabilizers and/or other active agents.

The use of an anti-CD4 antibody to induce tolerance against an antigen(s) in a primate in accordance with the present invention provides tolerance against one or more antigens and the primate is capable of immunologically responding to other antigens. Thus, in this respect, the primate is made tolerant to one or more antigens, and the immune system is capable of providing an immune response against other foreign antigens whereby the primate is not immunocompromised.

In the preferred embodiment where tolerance is induced against a foreign antigen, each of the CD4 antibody (and optionally the CD8 inhibiting compound, alone or in combination with each other) is administered to the primate prior to, in conjunction with or subsequent to the foreign antigen being delivered to the primate. In a preferred embodiment, the primate is provided with the CD4 antibody and the CD8 inhibiting compound at a time such that both are present in the primate when the antigen(s) against which tolerance is to be induced is also present in the primate. In a particularly preferred embodiment, each of the CD4 antibody (or fragment thereof) and the CD8 inhibiting compound is delivered to the primate prior to the primate coming into contact with the foreign antigen(s) to which the primate is to be tolerized or within a few hours or less than one day thereafter.

In one embodiment, each of the CD4 antibody (and optionally the CD8 inhibiting compound is administered to the primate at least about 5, at least about 4, at least about 3, at least about 2, or at least about 1 day prior to the primate receiving the foreign antigen. In one embodiment, the anti-CD4 antibody is administered about 5 days prior to the primate receiving the foreign antigen.

As hereinabove indicated, in one embodiment, a primate is tolerized against a therapeutic protein that is to be used in treating the primate. Such therapeutic protein may be a soluble antigen, e.g., a therapeutic antibody (other than the CD4 antibody) (which therapeutic antibody may be a human antibody, humanized antibody, chimeric antibody or a non-human antibody); an enzyme such as one used for replacement therapy; a hormone; clotting factor; a protein produced in gene therapy; a gene therapy delivery vehicle such as a vector used in gene therapy (for example, an adenovirus vector); or other soluble protein. As used herein, the term “soluble” includes antigens which are not cell bound (such as proteins which are naturally secreted by cells or which have been engineered to be soluble, e.g., by removal of transmembrane and cytoplasmic domains and/or by incorporation of various domains, e.g., antibody Fc region domains.

The foreign antigen(s) may be present in a transplanted organ, or in transplanted cells used in cell therapy, or in other tissue transplants, such as skin.

In one embodiment, the primate has not been exposed to the antigen prior to treatment with anti-CD4 antibody. In another embodiment, the primate has been exposed to the antigen prior to treatment with anti-CD4 antibody.

The treatment of a primate, in particular, a human, in order to tolerize the primate against a foreign antigen by use of a CD4 antibody and a CD8 inhibiting compound may be accomplished in some cases without adjunct therapy, such as a bone marrow transplant to promote acceptance of a foreign antigen and/or immunosuppression.

In some cases, adjunct therapy may also be employed. For example, as part of a transplant procedure, immunosuppression with an appropriate immunosuppressant may be used but by employing the present invention, chronic immunosuppression is not required. In addition, if used after or during the tolerizing procedure, in some cases, the immunosuppressant may be used with less than the amount required to provide for effective immunosuppression.

In one non-limiting embodiment, the CD4 antibody is preferably a TRX1 antibody or one that binds to the same epitope as TRX1, and such CD4 antibody is preferably used with the dosing regimen as hereinabove described.

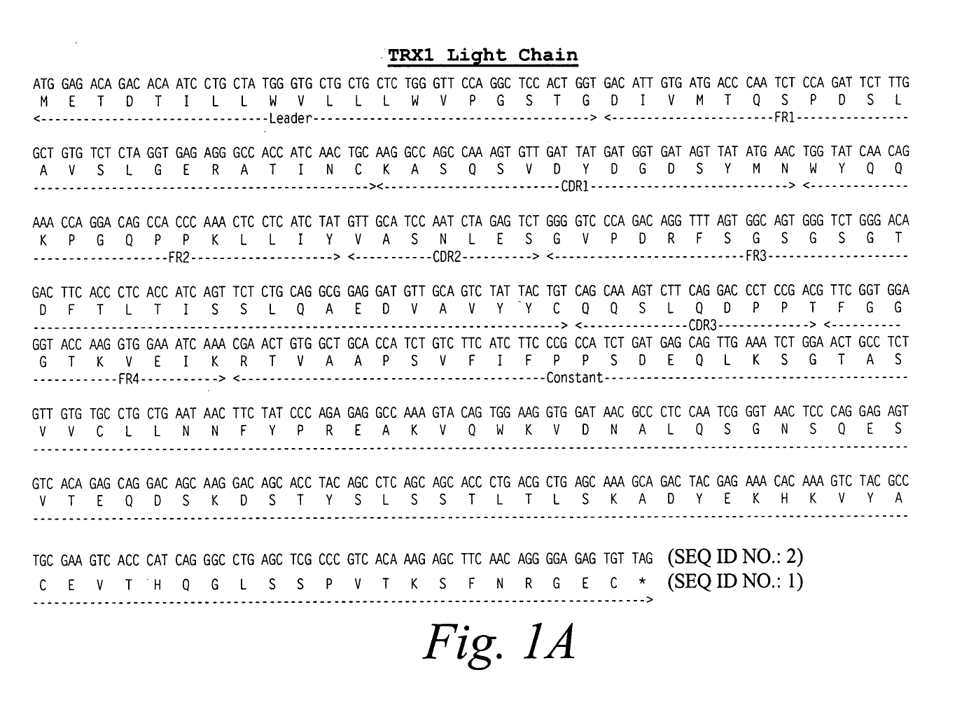

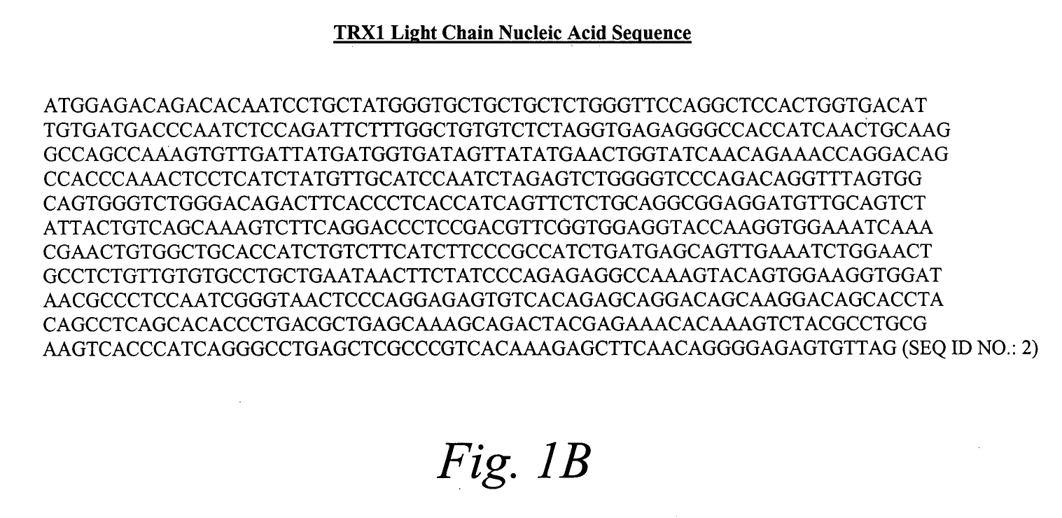

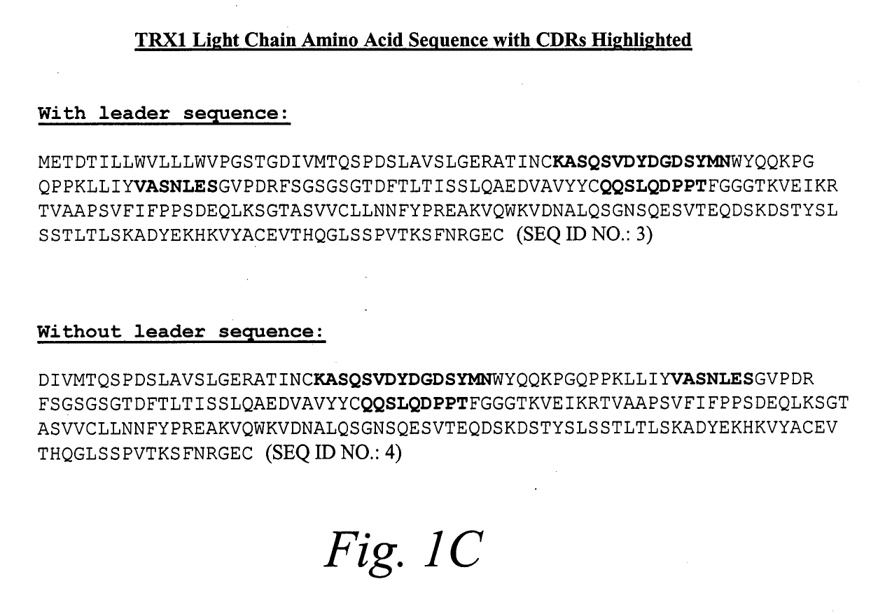

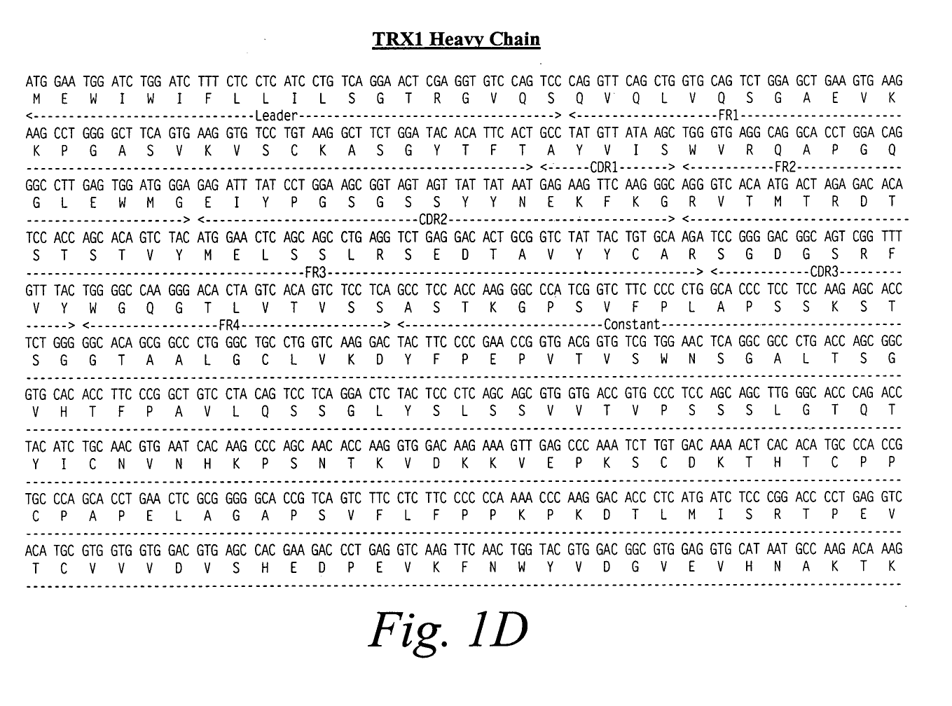

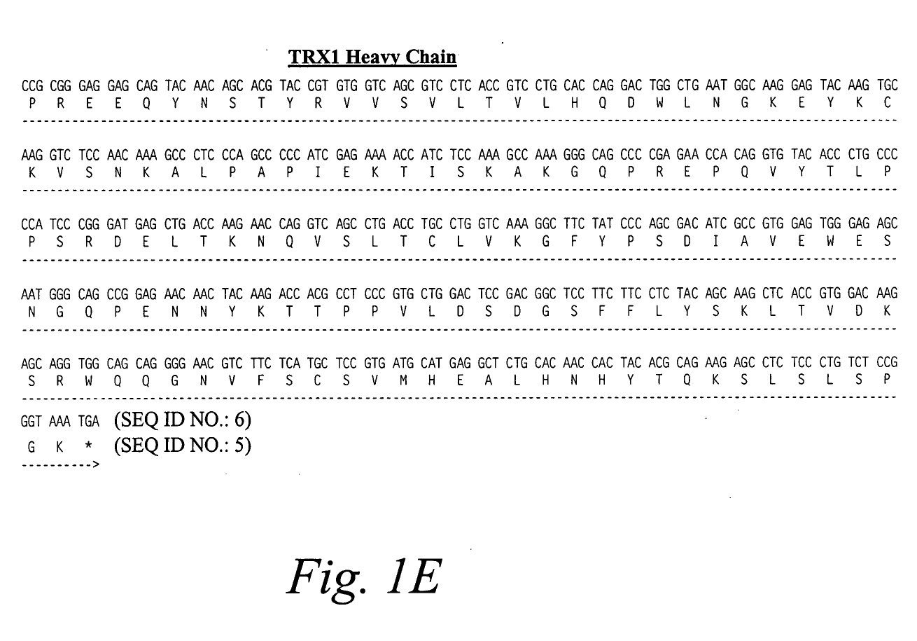

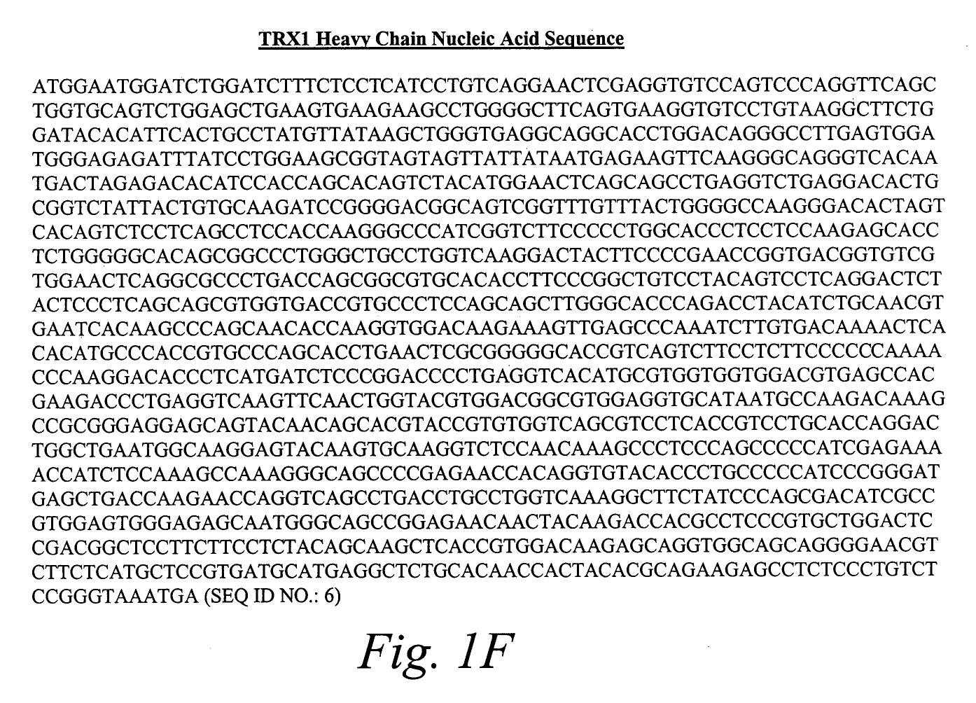

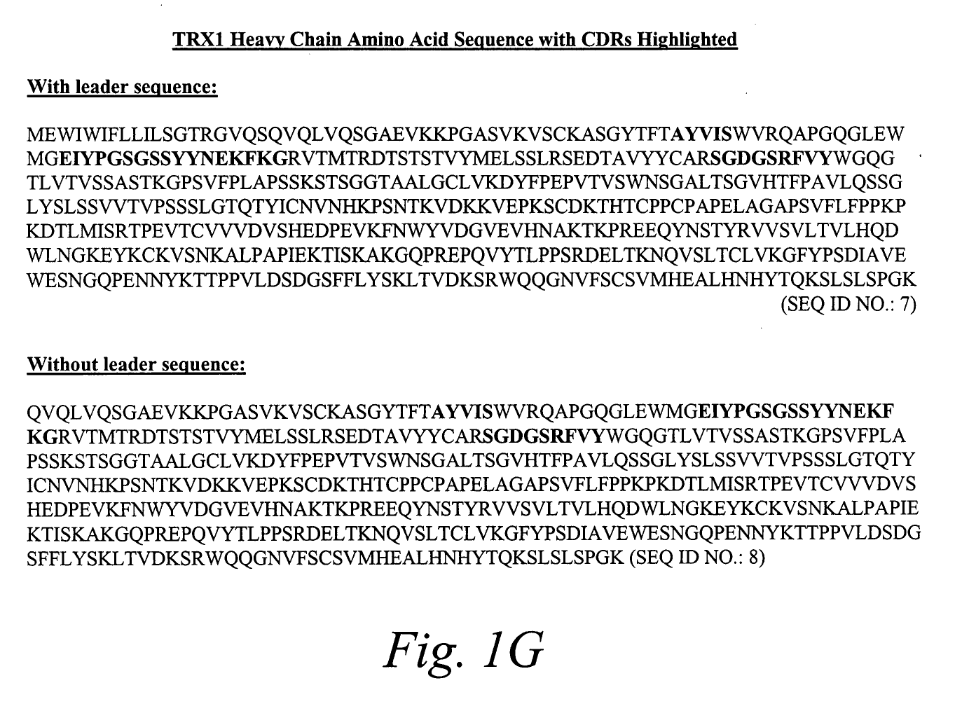

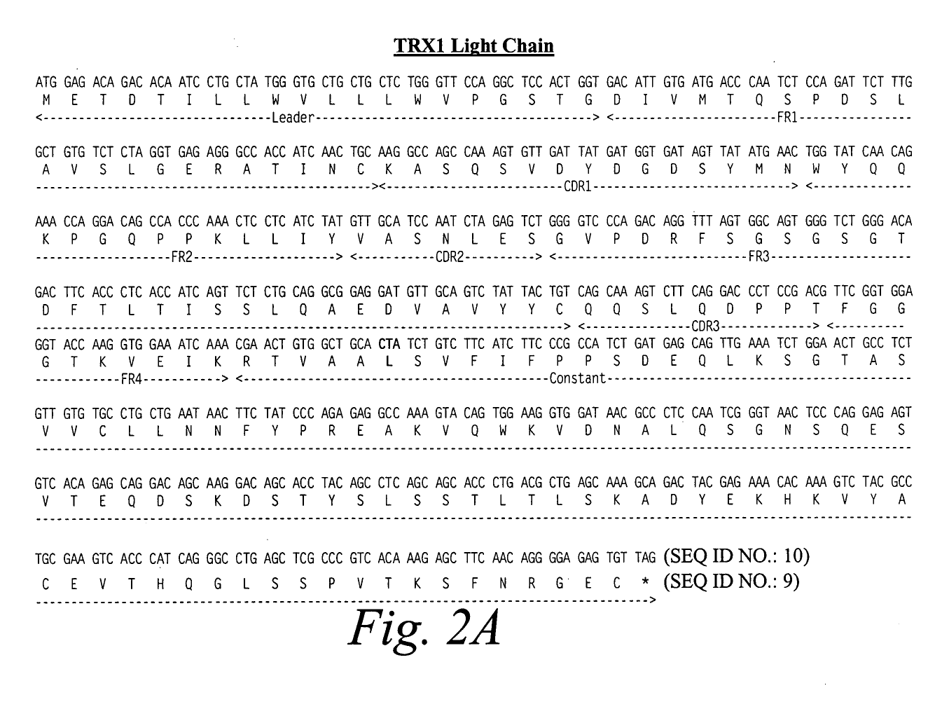

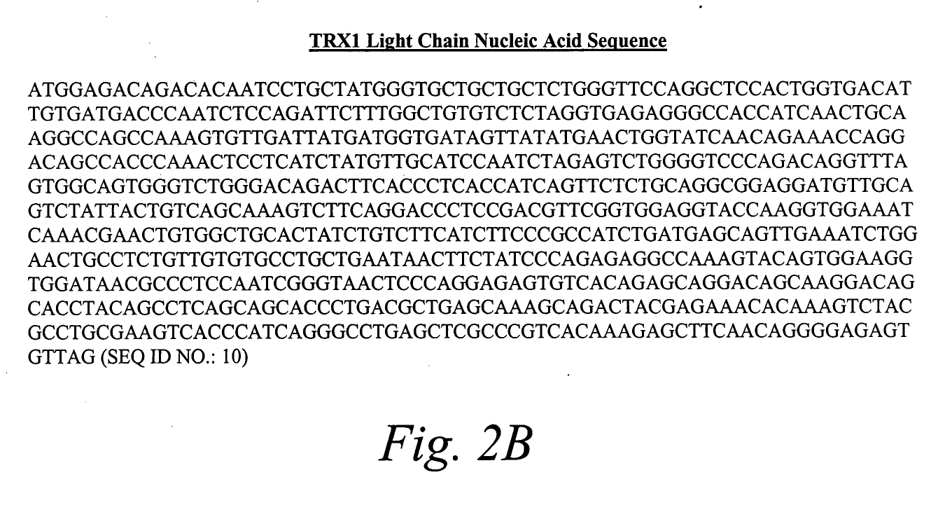

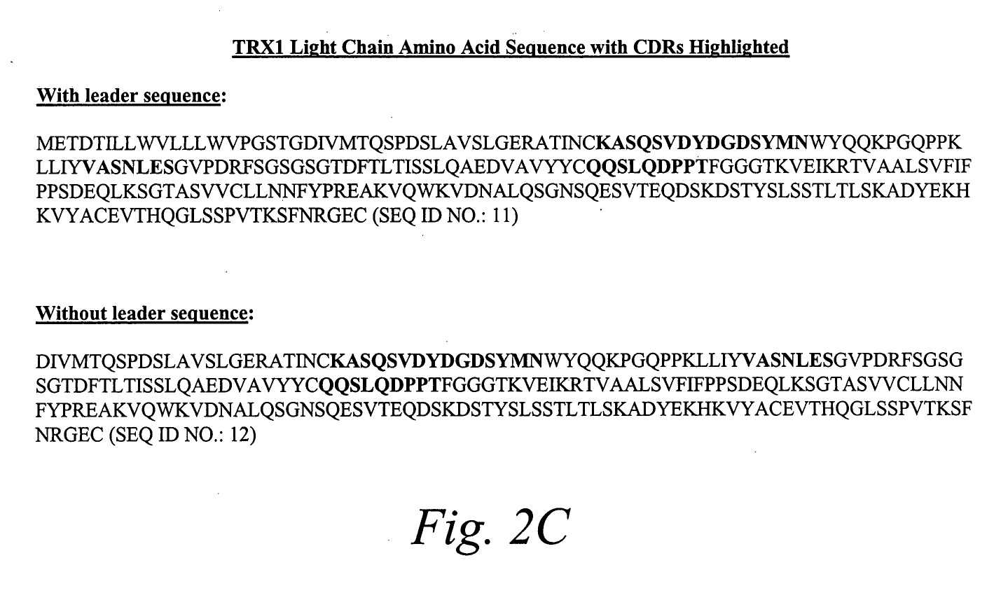

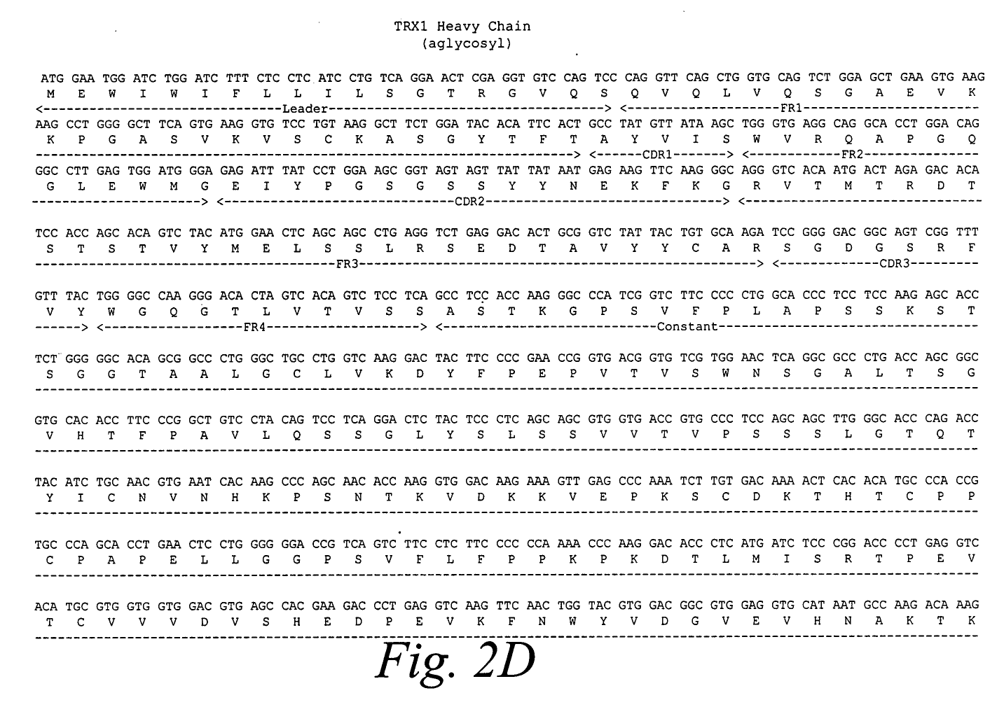

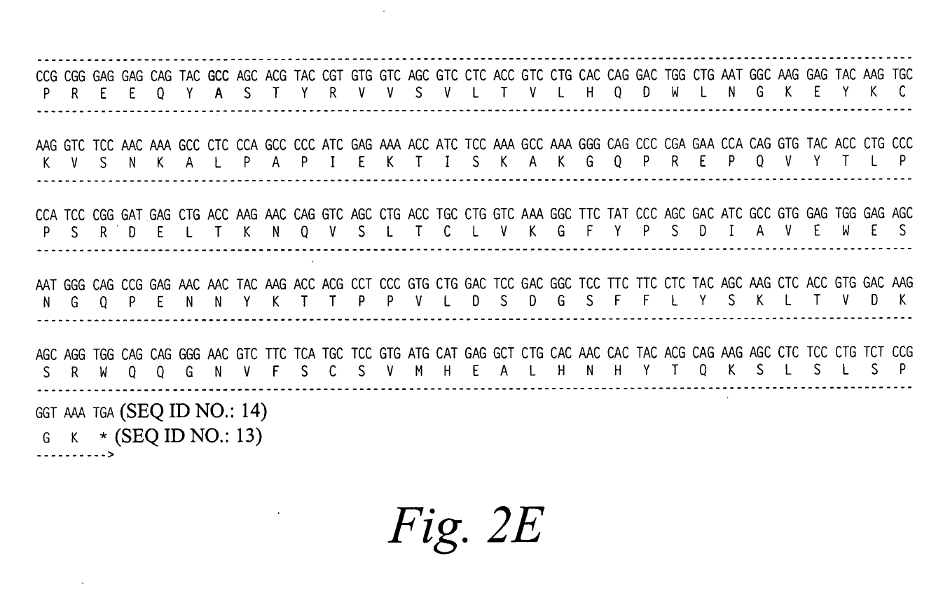

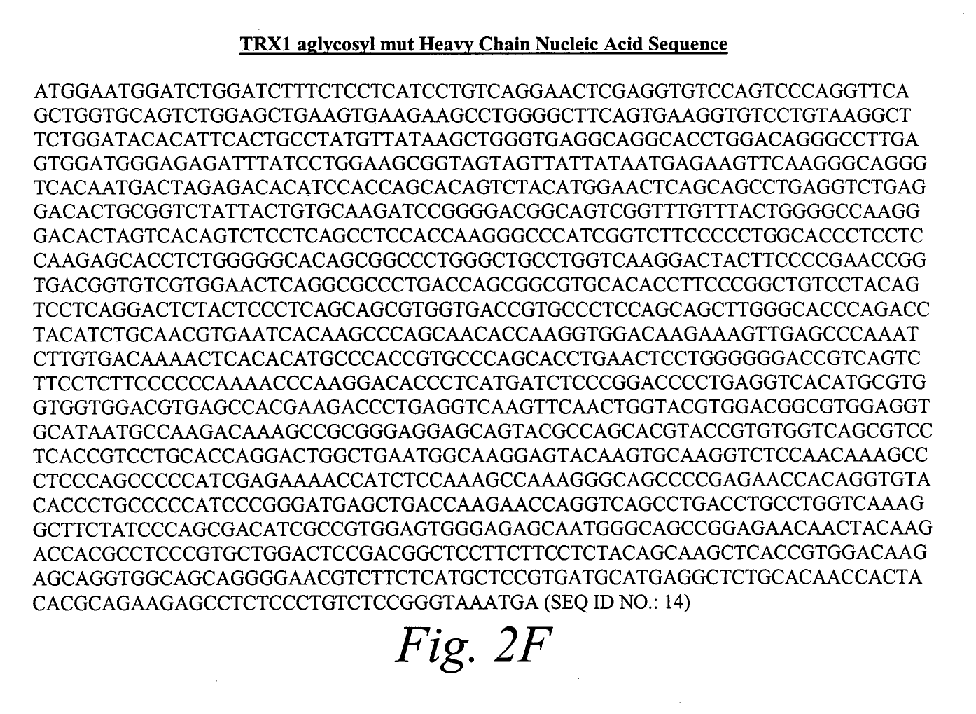

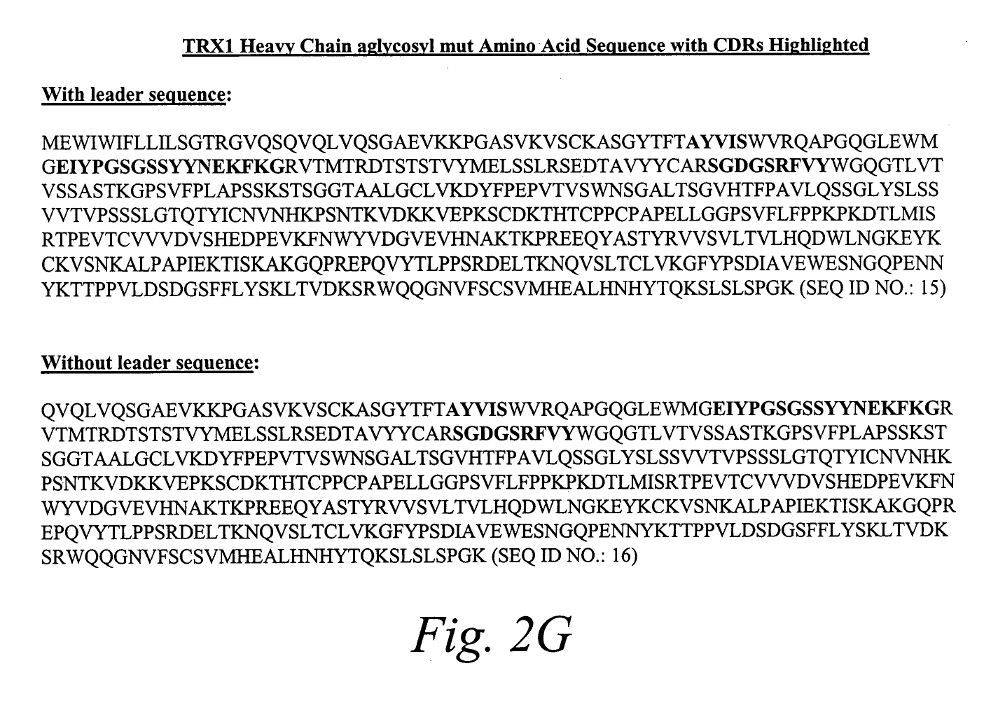

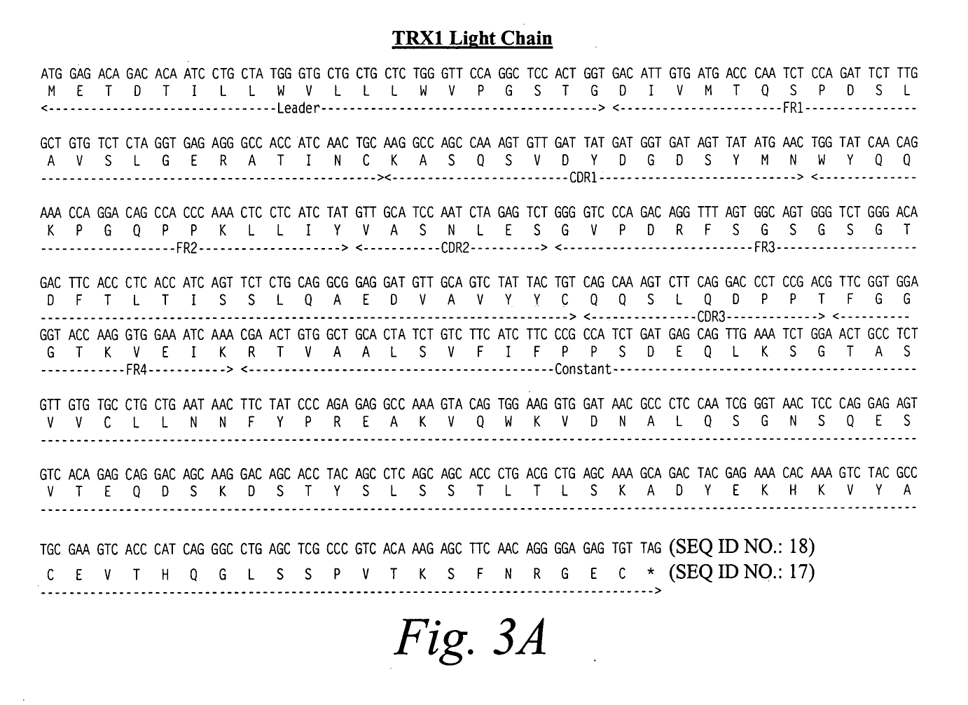

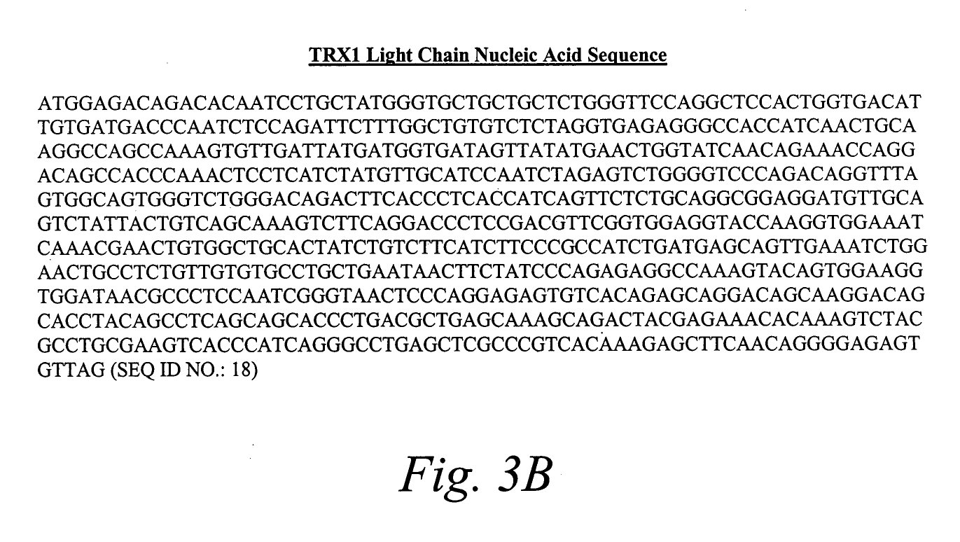

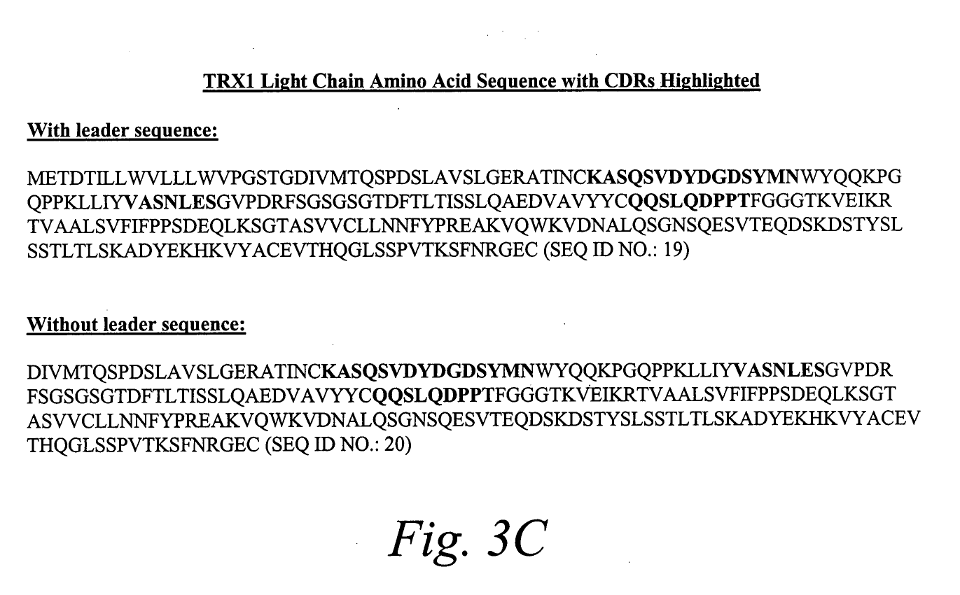

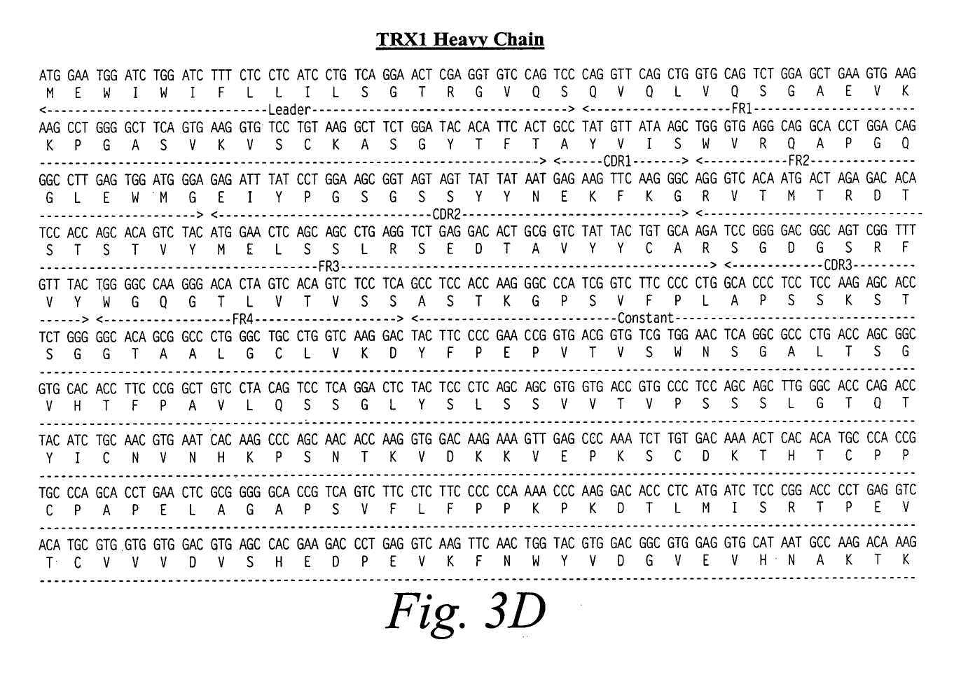

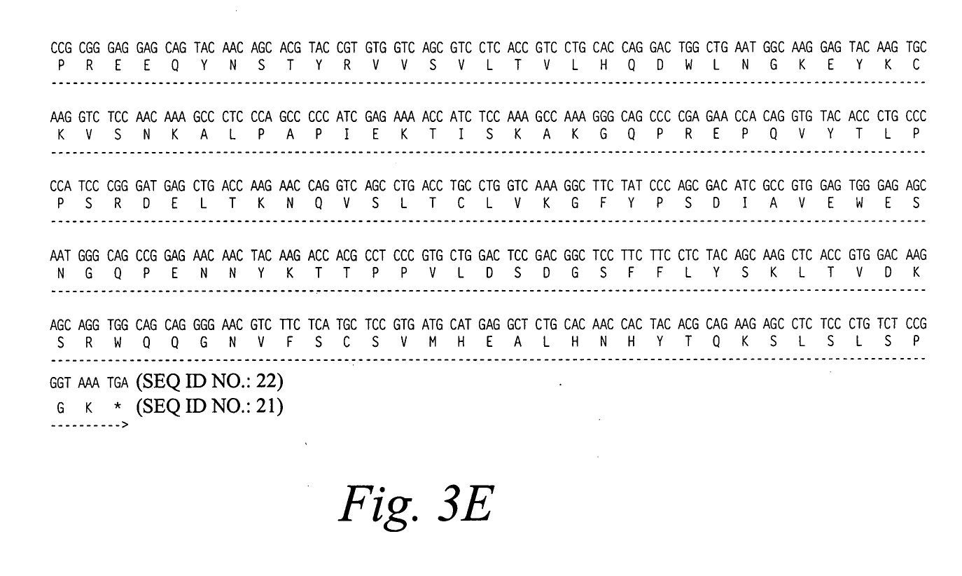

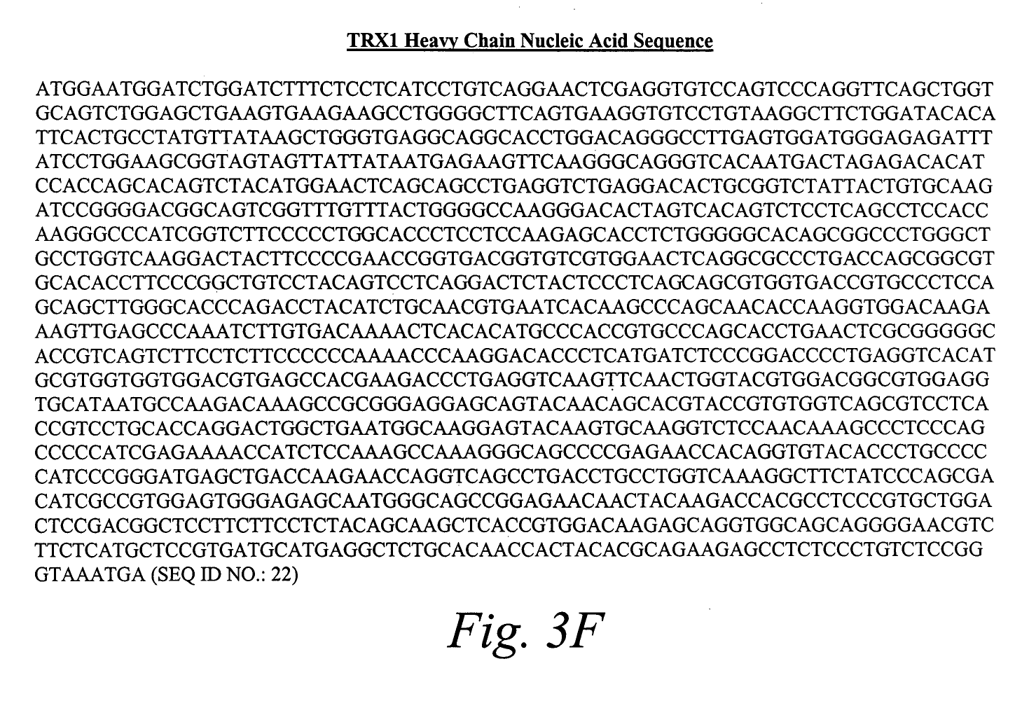

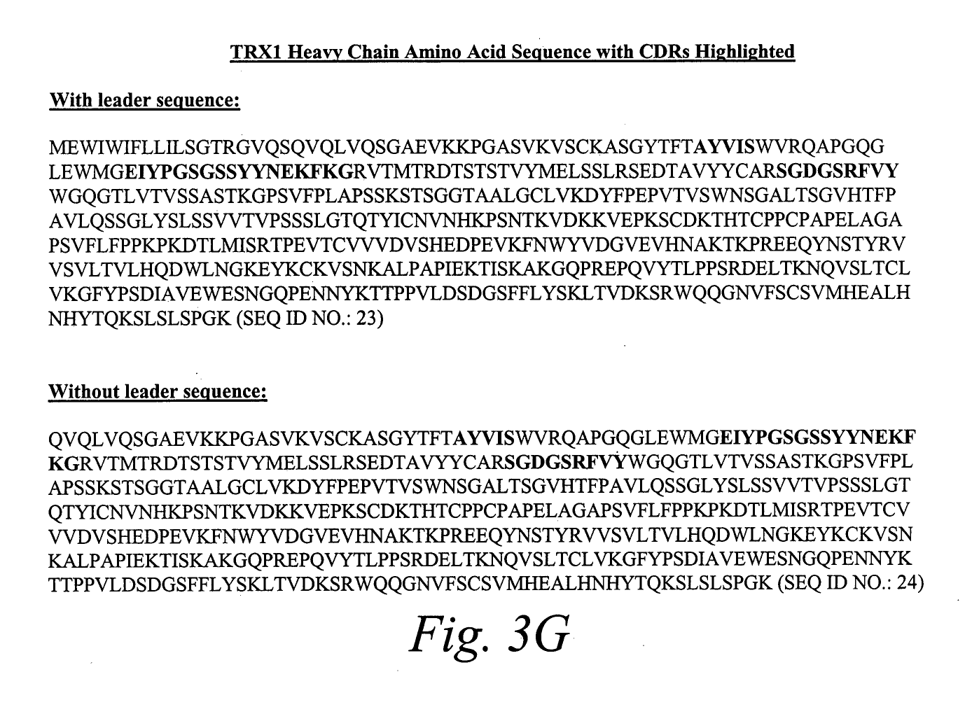

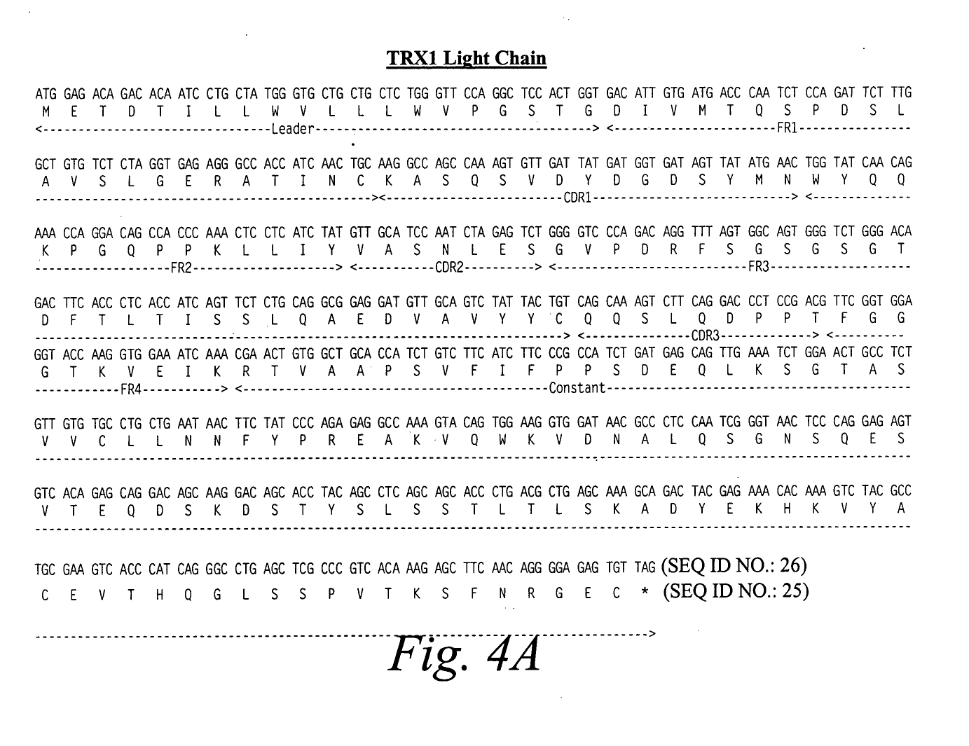

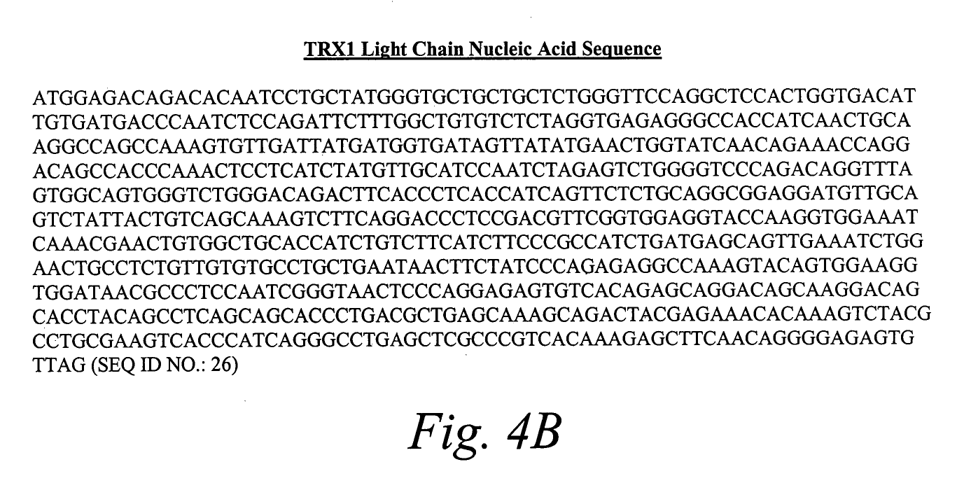

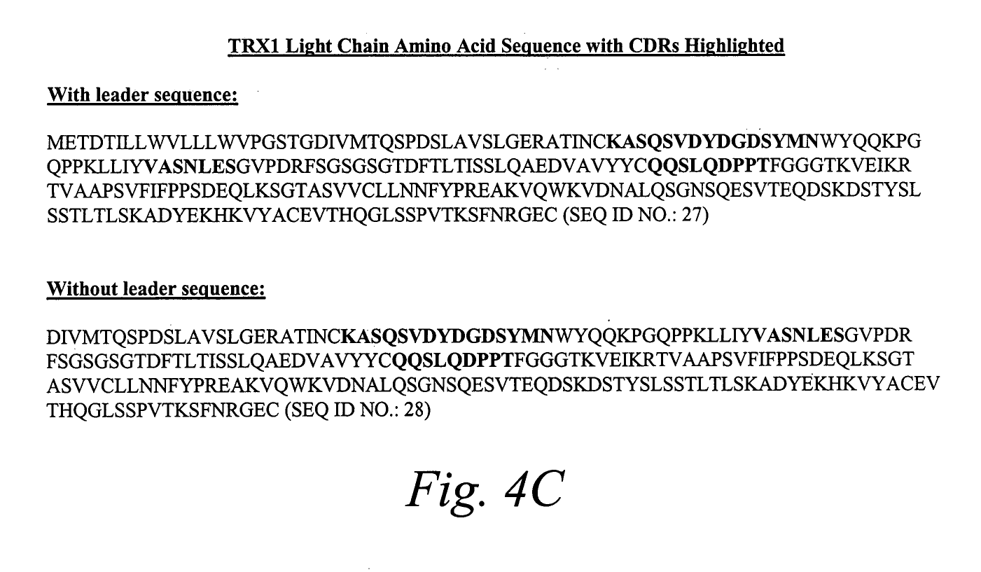

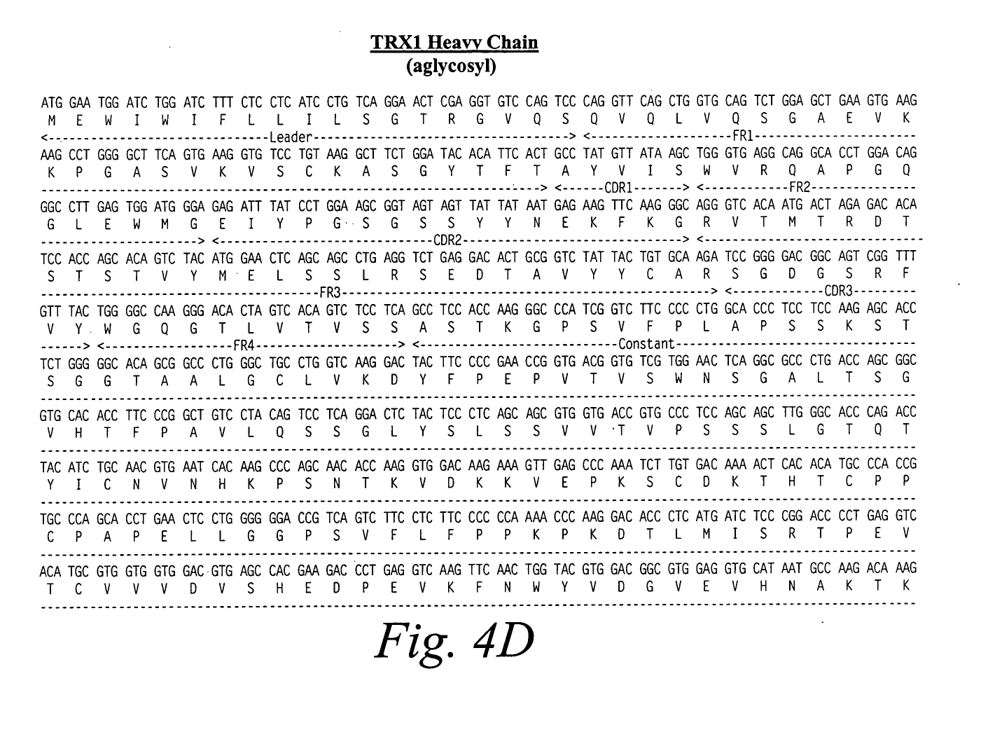

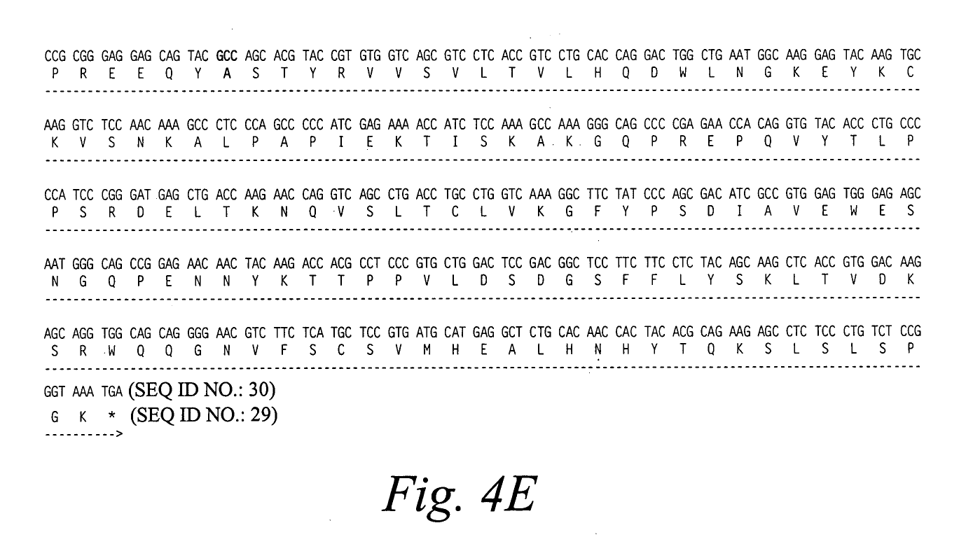

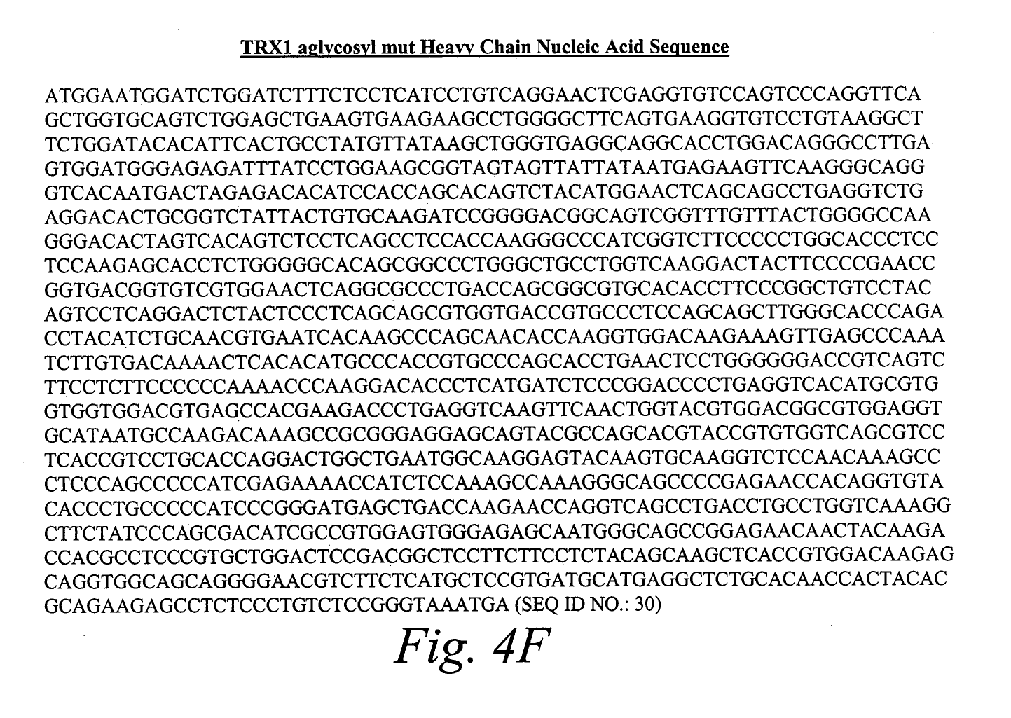

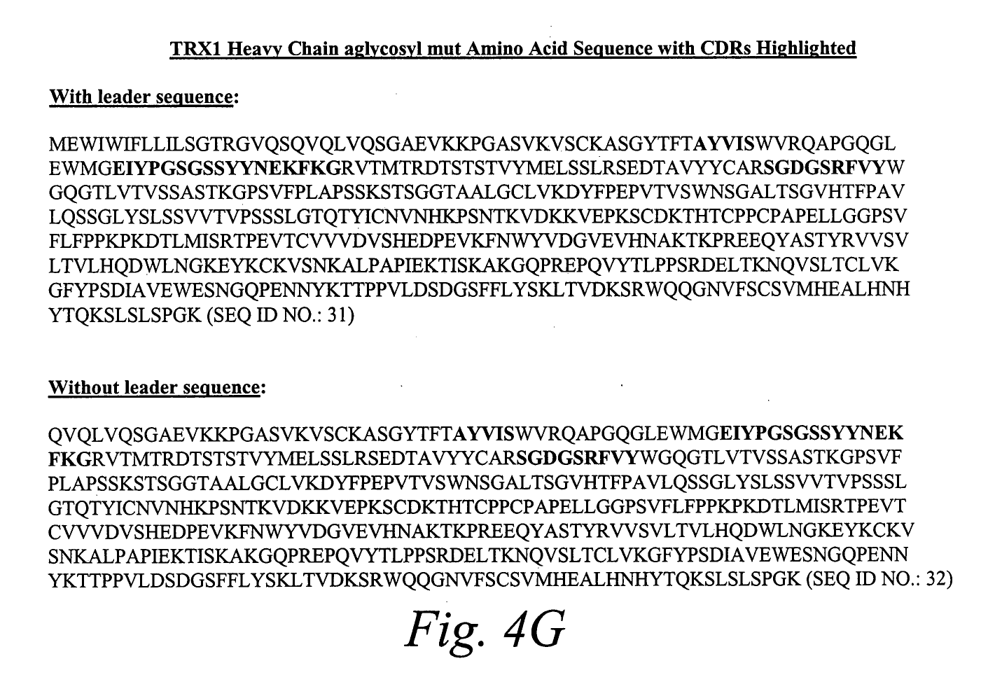

In accordance with an aspect of the present invention, such CD4 antibody (preferably a humanized antibody or fragment thereof) binds to the same epitope (or a portion thereof) on human lymphocytes as the humanized antibody selected from the group consisting of, the TRX1 humanized antibody, e.g., the components of which, e.g., light chain and heavy chain, each containing constant regions and variable regions, are depicted in FIGS. 1A-1G and correspond to SEQ ID Nos.: 1, 2, 3, 4, 5, 6, 7 and 8; the TRX1 humanized antibody, e.g. the components of which, e.g., light chain and heavy chain, each containing constant regions and variable regions, are depicted in FIGS. 2A-2G and correspond to Seq ID Nos.: 9, 10, 11, 12, 13, 14, 15, and 16; the TRX1 humanized antibody, e.g., the components of which, e.g., light chain and heavy chain, each containing constant regions and variable regions, are depicted in FIGS. 3A-3G and correspond to Seq ID Nos.: 17, 18, 19, 20, 21, 22, 23, and 24; and the TRX1 humanized antibody, e.g., the components of which, e.g., light chain and heavy chain, each containing constant regions and variable regions, are depicted in FIGS. 4A-4G and correspond to Seq ID Nos.: 25, 26, 27, 28, 29, 30, 31, and 32.

The antibody is hereinafter sometimes referred to as TRX1. The term “molecule” or “antibody that binds the same epitope as TRX1” includes TRX1. The term “TRX1” includes the components of the humanized antibody, e.g. light chain and heavy chain, each containing a constant region and a variable region, e.g., amino acid sequences shown in Seq ID Nos.: 1, 3, 4, 5, 7 and 8 (FIGS. 1A, 1C, 1D, 1E, and 1G), the components of the humanized antibody, e.g., light chain and heavy chain, each containing a constant region and a variable region, e.g., amino acid sequences shown in Seq ID Nos.: 9, 11, 12, 13, 15, and 16 (FIGS. 2A, 2C, 2D, 2E, and 2G), the components of the humanized antibody, e.g., light chain and heavy chain, each containing a constant region and a variable region, e.g., amino acid sequences shown in Seq ID Nos.: 17, 19, 21, 23, and 24 (FIGS. 3A, 3C, 3D, 3E, and 3G), the components of the humanized antibody, e.g., light chain and heavy chain, each containing a constant region and a variable region, e.g., amino acid sequences shown in Seq ID Nos.: 25, 27, 28, 29, 31, and 32 (FIGS. 4A, 4C, 4D, 4E, and 4G), and those identical thereto which may be produced, for example, by recombinant technology.

Although the preferred CD4 antibody is TRX1, from the teachings herein, other anti-CD4 antibodies can also be employed in the methods of the invention. For example, in one embodiment, one skilled in the art can produce antibodies that are equivalent to TRX1. For example, such antibodies may be:
1) humanized antibodies that bind to CD4 (e.g., by binding to the same epitope as TRX1);
2) humanized antibodies that have the same CDRs as TRX1 but which have a different humanized framework and/or a different human constant region;
3) humanized antibodies that bind to CD4 (e.g., by binding to the same epitope as TRX1) in which one or more amino acids of one or more of the CDRs of TRX1 have been changed (preferably but not necessarily a conservative amino acid substitution) and in which the framework may be the same framework as TRX1 or have a different humanized framework or in which one or more of the amino acids of the framework region of TRX1 have been changed and/or in which the constant region may be the same as or different from TRX1;
4) humanized antibodies that bind to CD4 (e.g., by binding to the same epitope as TRX1) wherein the antibody does not bind to Fc receptors through the Fc region of the antibody.
5) humanized antibodies that bind to CD4 (e.g., by binding to the same epitope as TRX1) wherein the CDRs thereof do not include a glycosylation site;
6) humanized antibodies that bind to CD4 (e.g., by binding to the same epitope as TRX1) and that do not bind to Fc receptors through the Fc region of the antibody and the CDRs do not include a glycosylation site;
7) a chimeric antibody that bind to CD4 (e.g., by binding to the same epitope as TRX1); and
8) a murine antibody that bind to CD4 (e.g., by binding to the same epitope as TRX1).

The antibodies that are equivalent to TRX1 may be used in the same manner and for the same purposes as TRX1.

In a preferred embodiment, the CD4 antibody employed in the present invention is one which binds to the same epitope (or a part of that epitope) as the TRX1 humanized antibody. The term “binds to the same epitope as TRX1 humanized antibody” is intended to describe not only the TRX1 humanized antibody but also describes other antibodies, fragments or derivatives thereof that bind to the same such epitope as the TRX1 humanized antibody. Antibodies that bind to the same epitope as TRX1 humanized antibody can be identified using techniques known to those of ordinary skill in the art, e.g., antibody competition assays or epitope mapping.

In a preferred embodiment, the CD4 antibody does not bind to Fc receptors through the Fc region of the antibody and the CDRs do not include a glycosylation site.

The constant region may or may not include a glycosylation site. In one embodiment, the constant region includes a glycosylation site. Glycosylation signals are well known in the art. An example of a heavy chain sequence which includes a glycosylation site is shown in SEQ ID NO.:5 (FIGS. 1D and 1E), SEQ ID NO.:7 (FIG. 1G) and SEQ ID NO.:8 (FIG. 1G), and SEQ ID NO.:21 (FIGS. 3D and 3E), SEQ ID NO.:23 (FIG. 3G) and SEQ ID NO.:24 (FIG. 3G). In another embodiment, the constant region does not include a glycosylation site due to an asparagine (N) to an alanine (A) amino acid change. An example of a heavy chain sequence which does not include a glycosylation site is shown in SEQ ID NO.: 13 (FIGS. 2D and 2E), SEQ ID NO.:15 (FIG. 2G) and SEQ ID NO.:16 (FIG. 2G), and SEQ ID NO.: 29 (FIGS. 4D and 4E), SEQ ID NO.:31 (FIG. 4G) and SEQ ID NO.:32 (FIG. 4G).

Such other antibodies include, by way of example and not by limitation, rat, murine, porcine, bovine, human, chimeric, humanized antibodies, or fragments or derivatives thereof.

The term “fragment” as used herein means a portion of an antibody, by way of example, such portions of antibodies shall include but not be limited to CDR, Fab, scFv molecules or such other portions, which bind to the same epitope or any portion thereof as recognized by TRX1.

The term “antibody” as used herein includes polyclonal and monoclonal antibodies as well as antibody fragments and derivatives, as well as antibodies prepared by recombinant techniques, such as chimeric or humanized antibodies, single chain or bispecific antibodies which bind to the same epitope or a portion thereof as recognized by the humanized antibody TRX1. The term “molecules” includes by way of example and not limitation, peptides, oligonucleotides or other such compounds derived from any source which mimic the antibody or binds to the same epitope or a portion thereof as the antibody fragment or derivative thereof.

Another embodiment of the present invention provides for a method of treating a patient who is to receive or has received a graft transplant with an effective amount of (i) at least one member selected from the group consisting of TRX1 antibody, or an antibody, or derivative or fragment thereof that bind to the same epitope (or a portion thereof) as the TRX1 antibody and (ii) a CD8 inhibiting compound. The treatment is preferably effected with the whole or intact TRX1 antibody.

In one embodiment, the anti-CD4 antibody used in the methods of the invention is humanized and is modified to reduce effector function, e.g., by modification to reduce Fc receptor and/or complement binding using methods known in the art.

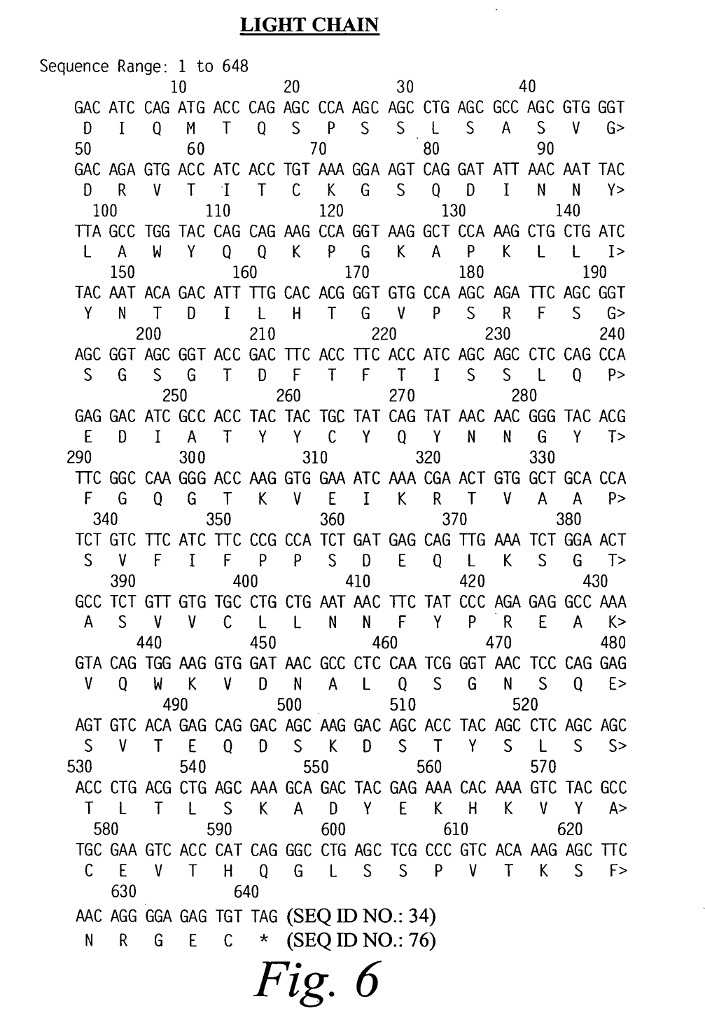

In one embodiment, the antibody is TRX1 (SEQ ID Nos.:1, 2, 3, 4, 5, 6, 7, and 8; FIGS. 1A, 1B, 1C, 1D, 1E, 1F, and 1G). The TRX1 antibody, e.g., the components of the TRX1 antibody, e.g., the light chain and heavy chain, each containing variable and constant regions, which are shown in, e.g., SEQ ID Nos.: 1 (FIG. 1A), 2, (FIG. 1B), 3 (FIG. 1C, top), 4 (FIG. 1C, bottom), 5 (FIGS. 1D and 1E), 6 (FIG. 1F), 7 (FIG. 1G, top), and 8 (FIG. 1G, bottom). SEQ ID No.:1 (FIG. 1A) is the amino acid sequence of the TRX1 light chain and SEQ ID No.:2 (FIG. 1B) is the nucleotide sequence of the TRX1 light chain. SEQ ID No.:3 (FIG. 1C, top) is the amino acid sequence of the TRX1 light chain, with a leader sequence. SEQ ID No.:4 (FIG. 1C, bottom) is the amino acid sequence of the TRX1 light chain, e.g., SEQ ID No.:1 or SEQ ID No.:3, without a leader sequence, e.g., amino acid residues 1-20 of SEQ ID No.:1. The TRX1 heavy chain amino acid sequence, containing a glycosylation site, e.g., amino acid residues 317-319, is shown in SEQ ID No.:5 (FIGS. 1D and 1E) and the nucleotide sequence of the TRX1 heavy chain is shown in SEQ ID No.:6 (FIG. 1F). SEQ ID No.:7 (FIG. 1G, top) is the amino acid sequence of the TRX1 heavy chain with a leader sequence. SEQ ID No.:8 (FIG. 1G, bottom) is the amino acid sequence of the TRX1 heavy chain, e.g., SEQ ID No.:5 (FIGS. 1D and 1E), without a leader sequence, e.g., amino acid residues 1-19 of SEQ ID No.:5 (FIGS. 1D and 1E), and contains a glycosylation site, e.g., amino acid residues 298-300. TRX1 is a humanized antibody that includes modified constant regions of a human antibody, e.g., light chain amino acid residues 132-238 of SEQ ID No.:1 (FIG. 1A) or SEQ ID No.:3 (FIG. 1C, top), and amino acid residues 112-218 of SEQ ID No.:4 (FIG. 1C, bottom), and heavy chain amino acid residues 138-467 of SEQ ID No,:5 (FIGS. 1D and 1E) or SEQ ID No.:7 (FIG. 1G, top) and amino acid residues 119-448 of SEQ ID No.:8 (FIG. 1G), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:1 (FIG. 1A) or SEQ ID No.:3 (FIG. 1C, top) and amino acid residues 1-22, 33-53, 61-92, and 102-111 of SEQ ID No.:4 (FIG. 1C), and framework regions of the heavy chain variable region, e.g., amino acid residues 20-49, 55-68, 86-117, and 127-137 of SEQ ID No.:5 or SEQ ID No.:7 (FIG. 1G, top) and amino acid residues 1-30, 36-49, 67-98, and 108-118 of SEQ ID No.:8, which are derived from a human antibody, and the CDRs of the light chain, e.g., amino acid residues 44-58, 74-80, and 113-121 of SEQ ID No.:1 or SEQ ID No.:3 (FIG. 1C, top), and amino acid residues 24-32, 54-60, and 93-101 of SEQ ID No.:4, and the CDRs of the heavy chain, e.g., amino acid residues 50-54, 69-85, and 118-126 of SEQ ID No.:5 or SEQ ID No.:7 (FIG. 1G, top) and amino acid residues 31-35, 50-66, and 99-107 of SEQ ID No.:8, which are derived from a mouse monoclonal antibody designated NSM4.7.2.4.

In another embodiment, the antibody is TRX1 (SEQ ID Nos.:17, 18, 19, 20, 21, 22, 23, and 24; FIGS. 3A, 3B, 3C, 3D, 3E, 3F, and 3G). The TRX1 antibody, e.g., the components of the TRX1 antibody, e.g., the light chain and heavy chain, each containing variable and constant regions, are shown in, e.g., SEQ ID Nos.: 17 (FIG. 3A), 18, (FIG. 3B), 19 (FIG. 3C, top), 20 (FIG. 3C, bottom), 21 (FIGS. 3D and 3E), 22, (FIG. 3F) 23 (FIG. 3G, top), and 24 (FIG. 3G, bottom). SEQ ID No.:17 (FIG. 3A) is the amino acid sequence of the TRX1 light chain and SEQ ID No.:18 (FIG. 3B) is the nucleotide sequence of the TRX1 light chain. SEQ ID No.:19 (FIG. 3C, top) is the amino acid sequence of the TRX1 light chain with a leader sequence. SEQ ID No.:20 (FIG. 3C, bottom) is the amino acid sequence of the TRX1 light chain, e.g., SEQ ID No.:17, without a leader sequence, e.g., amino acid residues 1-20 of SEQ ID No.:17. The TRX1 heavy chain amino acid sequence, containing a glycosylation site, e.g., amino acid residues 317-319 of SEQ ID No.:21 (FIGS. 3D and 3E) and the nucleotide sequence of the TRX1 heavy chain is shown in SEQ ID No.:22 (FIG. 3F). SEQ ID No.:23 (FIG. 3G, top) is the amino acid sequence of the TRX1 heavy chain with a leader sequence. SEQ ID No.:24 (FIG. 3G, bottom) is the amino acid sequence of the TRX1 heavy chain, e.g., SEQ ID No.:21, without a leader sequence, e.g., amino acid residues 1-19 of SEQ ID No.:21, and contains a glycosylation site, e.g., amino acid residues 298-300. TRX1 is a humanized antibody that includes modified constant regions of a human antibody, e.g., light chain amino acid residues 132-238 of SEQ ID No.:17 (FIG. 3A) or SEQ ID No.:19 (FIG. 3C, top), and amino acid residues 112-218 of SEQ ID No.:20 (FIG. 3C, bottom), and heavy chain amino acid residues 138-467 of SEQ ID No.:21 (FIGS. 3D and 3E) or SEQ ID No.:23 (FIG. 3G, top) and amino acid residues 119-448 of SEQ ID No.:24 (FIG. 3G, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:17 (FIG. 3A) or SEQ ID No.:19 (FIG. 3C, top), and amino acid residues 1-22, 33-53, 61-92, and 102-111 of SEQ ID No.:20, and framework regions of the heavy chain variable region, e.g., amino acid residues 2049, 55-68, 86-117, and 127-137 of SEQ ID No.:21 (FIGS. 3D and 3E) or SEQ ID No.:23 (FIG. 3G, top) and amino acid residues 1-30, 36-49, 67-98, and 108-118 of SEQ ID No.:24 (FIG. 3G, bottom), which are derived from a human antibody, and the CDRs of the light chain, e.g., amino acid residues 44-58, 74-80, and 113-121 of SEQ ID No.:17 (FIG. 3A) or SEQ ID No.:19 (FIG. 3C, top), and amino acid residues 24-32, 54-60, and 93-101 of SEQ ID No.:20 (FIG. 3C, bottom), and the CDRs of the heavy chain, e.g., amino acid residues 50-54, 69-85, and 118-126 of SEQ ID No.:21 (FIGS. 3D and 3E) or SEQ ID No.:23 (FIG. 3G, top) and amino acid residues 31-35, 50-66, and 99-107 of SEQ ID No.:24 (FIG. 3G, bottom), which are derived from a mouse monoclonal antibody designated NSM4.7.2.4.

In another embodiment, the antibody is TRX1 (SEQ ID Nos.:9, 10, 11, 12, 13, 14, 15, and 16; FIGS. 2A, 2B, 2C, 2D, 2E, 2F, and 2G). The TRX1 antibody, e.g., the components of the TRX1 antibody, e.g. the light chain and heavy chain, each containing variable and constant regions, are shown in, e.g. SEQ ID Nos.: 9 (FIG. 2A), 10, (FIG. 2B), 11 (FIG. 2C, top), 12 (FIG. 2C, bottom), 13 (FIGS. 2D and 2E), 14 (FIG. 2F), 15 (FIG. 2G, top) and 16 (FIG. 2G, bottom). SEQ ID No.:9 (FIG. 2A) is the amino acid sequence of the TRX1 light chain and SEQ ID No.:10 (FIG. 2B) is the nucleotide sequence of the TRX1 light chain. SEQ ID No.:11 (FIG. 2C) is the amino acid sequence of the TRX1 light chain with a leader sequence. SEQ ID No.:12 (FIG. 2C) is the amino acid sequence of the TRX1 light chain, e.g., SEQ ID No.:9 (FIG. 2A), without a leader sequence, e.g. amino acid residues 1-20 of SEQ ID No.:9. The TRX1 heavy chain amino acid sequence, which does not contain a glycosylation site, e.g., contains an asparagine to alanine change at amino acid residue 317, is shown in SEQ ID No.:13 (FIGS. 2D and 2E) and the nucleotide sequence of the TRX1 heavy chain is shown in SEQ ID No.:14 (FIG. 2F). SEQ ID No.:15 (FIG. 2G, top) is the amino acid sequence of the TRX1 heavy chain with a leader sequence. SEQ ID No.:16 (FIG. 2G, bottom) is the amino acid sequence of the TRX1 heavy chain, e.g., SEQ ID No.:13, without a leader sequence, e.g., amino acid residues 1-19 of SEQ ID No.:13, and does not contain a glycosylation site, e.g., contains an asparagine to alanine change at amino acid residue 298. TRX1 is a humanized antibody that includes modified constant regions of a human antibody, e.g., light chain amino acid residues 132-238 of SEQ ID No.:9 (FIG. 2A) or SEQ ID No.:11 (FIG. 2C, top), and amino acid residues 112-218 of SEQ ID No.:12 (FIG. 2C, bottom), and heavy chain amino acid residues 138-467 of SEQ ID No.:13 (FIGS. 2D and 2E) or SEQ ID No.:15 (FIG. 2G, top) and amino acid residues 119-448 of SEQ ID No.:16 (FIG. 2G, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:9 (FIG. 2A) or SEQ ID No.:11 (FIG. 2C, top), and amino acid residues 1-22, 33-53, 61-92, and 102-111 of SEQ ID No.:12 (FIG. 2C, bottom), and framework regions of the heavy chain variable region, e.g., amino acid residues 20-49, 55-68, 86-117, and 127-137 of SEQ ID No.:13 (FIGS. 2D and 2E) or SEQ ID No.:15 (FIG. 2G, top) and amino acid residues 1-30, 36-49, 67-98, and 108-118 of SEQ ID No.:16 (FIG. 2G, bottom), which are derived from a human antibody, and the CDRs of the light chain, e.g., amino acid residues 44-58, 74-80, and 113-121 of SEQ ID No.:9 (FIG. 2A) or SEQ ID No.:11 (FIG. 2C, top), and amino acid residues 24-32, 54-60, and 93-101 of SEQ ID No.:12 (FIG. 2C, bottom), and the CDRs of the heavy chain, e.g., amino acid residues 50-54, 69-85, and 118-126 of SEQ ID No.:13 (FIGS. 2D and 2E) or SEQ ID No.:15 (FIG. 2G, top) and amino acid residues 31-35, 50-66, and 99-107 of SEQ ID No.:16 (FIG. 2G, bottom), which are derived from a mouse monoclonal antibody designated NSM4.7.2.4.

In another embodiment, the antibody is TRX1 (SEQ ID Nos.:25, 26, 27, 28, 29, 30, 31, and 32; FIGS. 4A, 4B, 4C, 4D, 4E, 4F, and 4G). The TRX1 antibody, e.g., the components of the TRX1 antibody, e.g., the light chain and heavy chain, each containing variable and constant regions, are shown in, e.g., SEQ ID Nos.: 25 (FIG. 4A), 26 (FIG. 4B), 27 (FIG. 4C, top), 28 (FIG. 4C, bottom), 29 (FIGS. 4D and 4E), 30 (FIG. 4F), 31 (FIG. 4G, top), and 32 (FIG. 4G, bottom). SEQ ID No.:25 (FIG. 4A) is the amino acid sequence of the TRX1 light chain and SEQ ID No.:26 (FIG. 4B) is the nucleotide sequence of the TRX1 light chain. SEQ ID No.:27 (FIG. 4C, top) is the amino acid sequence of the TRX1 light chain with a leader sequence. SEQ ID No.:28 (FIG. 4C, bottom) is the amino acid sequence of the TRX1 light chain, e.g., SEQ ID No.:25, without a leader sequence, e.g., amino acid residues 1-20 of SEQ ID No.:25. The TRX1 heavy chain amino acid sequence, which does not contain a glycosylation site, e.g., contains an asparagine to alanine change at amino acid residue 317, is shown in SEQ ID No.:29 (FIGS. 4D and 4E) and the nucleotide sequence of the TRX1 heavy chain is shown in SEQ ID No.:30 (FIG. 4F). SEQ ID No.:31 (FIG. 4G, top) is the amino acid sequence of the TRX1 heavy chain with a leader sequence. SEQ ID No.:32 (FIG. 4G, bottom) is the amino acid sequence of the TRX1 heavy chain, e.g., SEQ ID No.:29, without a leader sequence, e.g., amino acid residues 1-19 of SEQ ID No.:29, and does not contain a glycosylation site, e.g., contains an asparagine to alanine change at amino acid residue 298. TRX1 is a humanized antibody that includes modified constant regions of a human antibody, e.g., light chain amino acid residues 132-238 of SEQ ID No.:25 (FIG. 4A) or SEQ ID No.:27 (FIG. 4C, top), and amino acid residues 112-218 of SEQ ID No.:28 (FIG. 4C, bottom), and heavy chain amino acid residues 138-467 of SEQ ID No.:29 (FIGS. 4D and 4E) or SEQ ID No.:31 (FIG. 4G, top) and amino acid residues 119-448 of SEQ ID No.:32 (FIG. 4G, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:25 (FIG. 4A) or SEQ ID No.:27 (FIG. 4C, top), and amino acid residues 1-22, 33-53, 61-92, and 102-111 of SEQ ID No.:28 (FIG. 4C, bottom), and framework regions of the heavy chain variable region, e.g., amino acid residues 20-49, 55-68, 86-117, and 127-137 of SEQ ID No.:29 (FIGS. 4D and 4E) or SEQ ID No.:31 (FIG. 4G, top) and amino acid residues 1-30, 36-49, 67-98, and 108-118 of SEQ ID No.:32 (FIG. 4G, bottom), which are derived from a human antibody, and the CDRs of the light chain, e.g., amino acid residues 44-58, 74-80, and 113-121 of SEQ ID No.:25 (FIG. 4A) or SEQ ID No.:27 (FIG. 4C, top), and amino acid residues 24-32, 54-60, and 93-101 of SEQ ID No.:28 (FIG. 4C, bottom), and the CDRs of the heavy chain, e.g., amino acid residues 50-54, 69-85, and 118-126 of SEQ ID No.:29 (FIGS. 4D and 4E) or SEQ ID No.:31 (FIG. 4G, top) and amino acid residues 31-35, 50-66, and 99-107 of SEQ ID No.:32 (FIG. 4G, bottom) which are derived from a mouse monoclonal antibody designated NSM4.7.2.4.

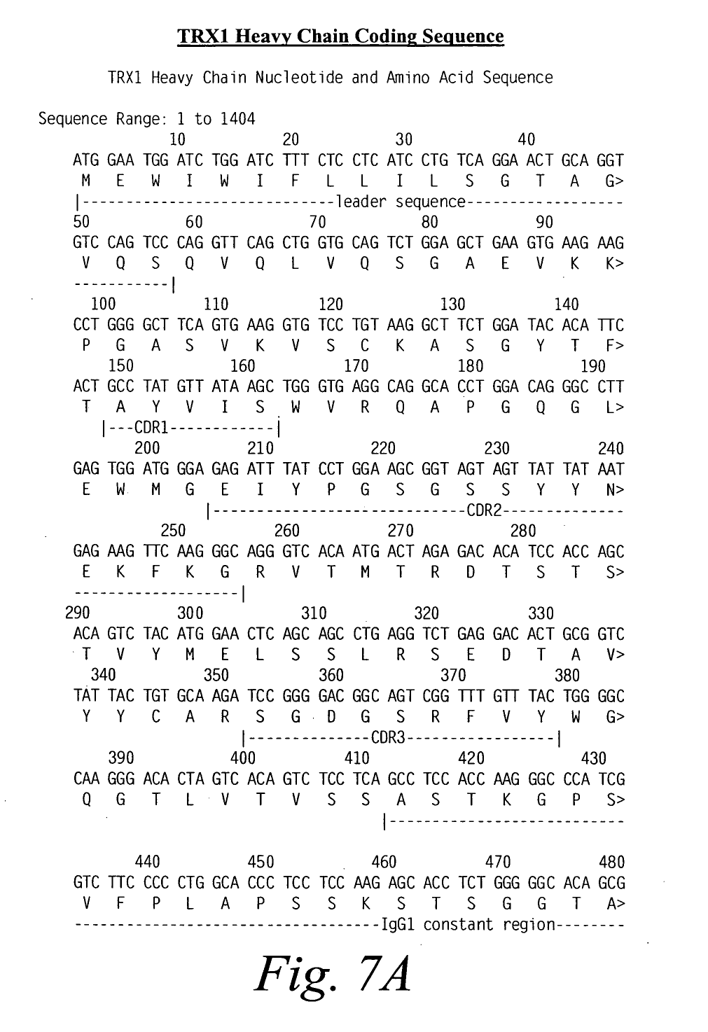

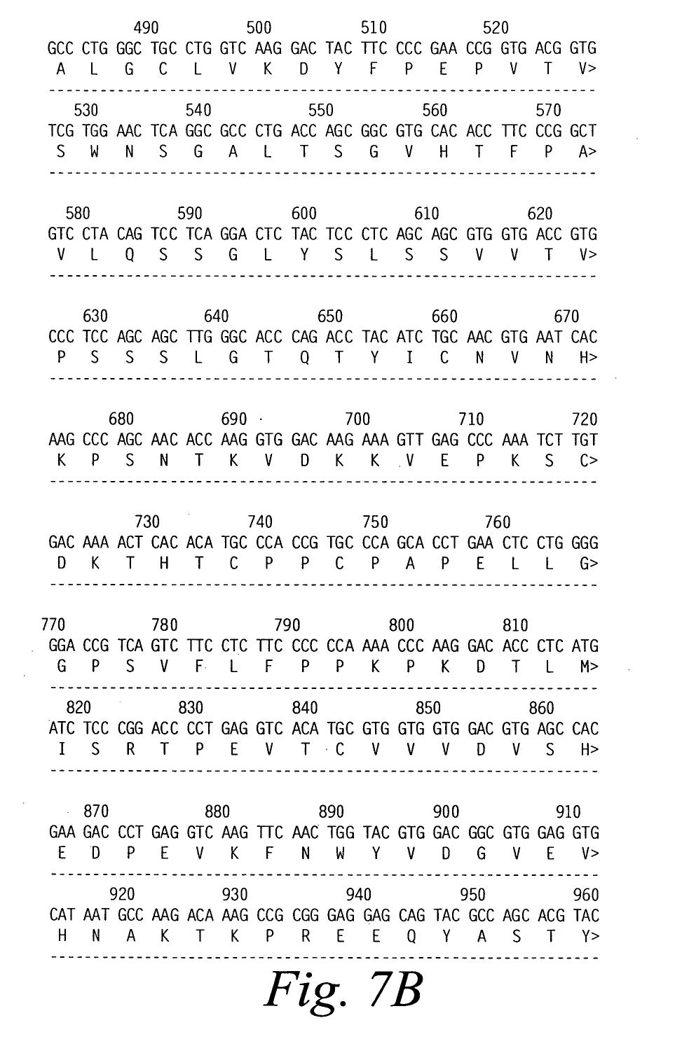

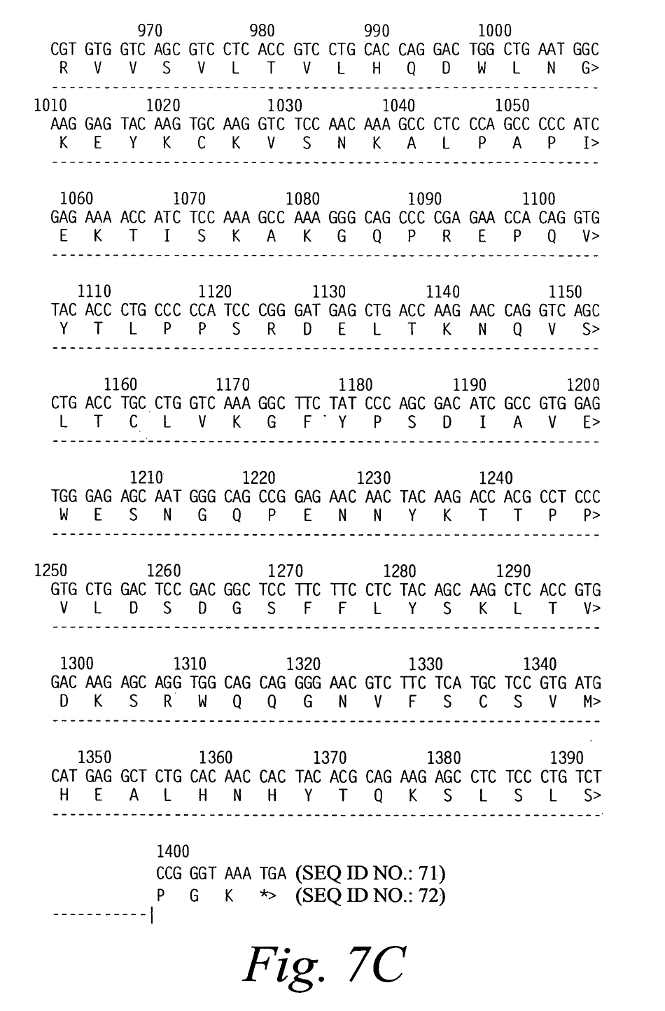

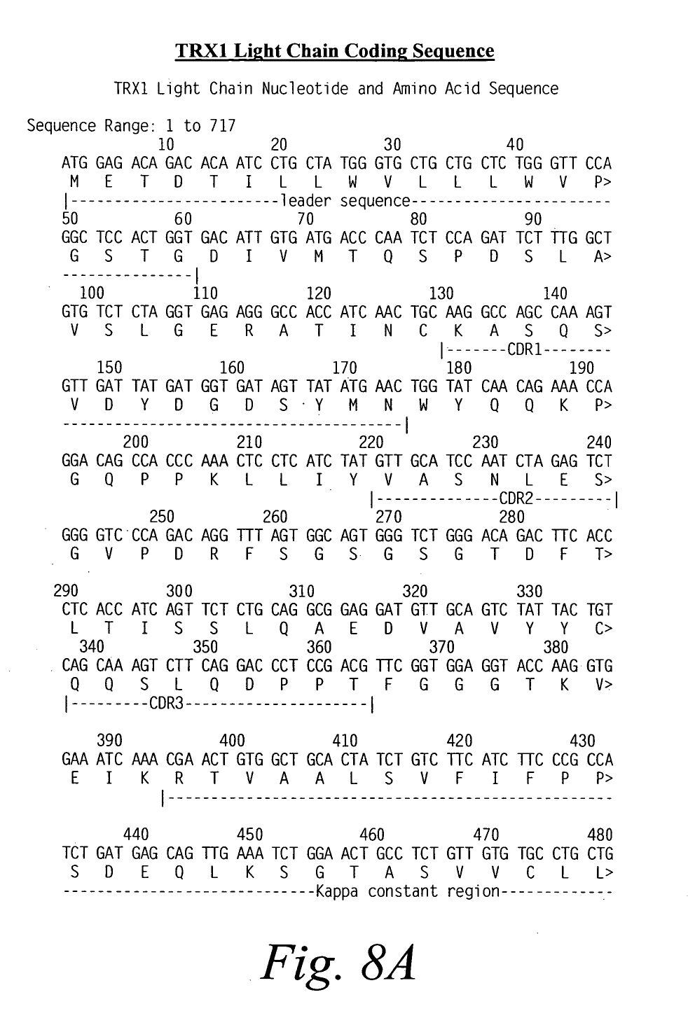

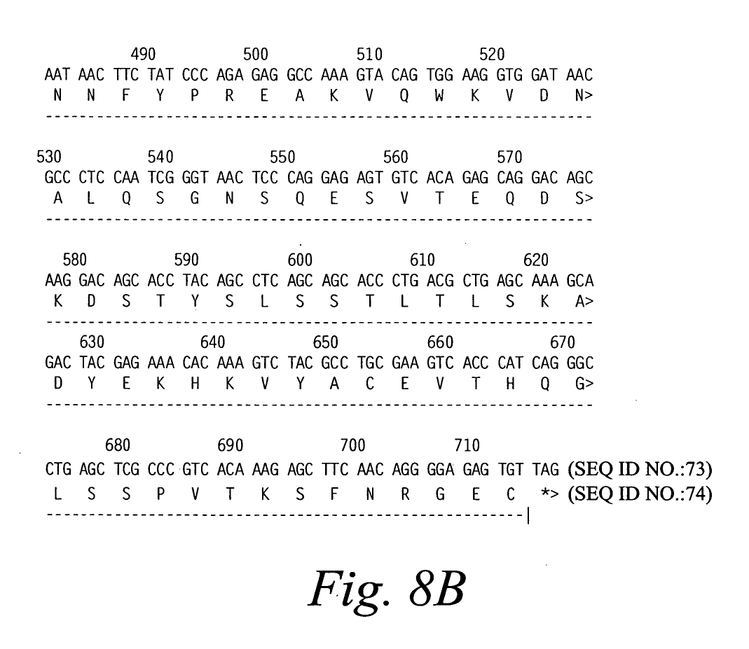

In another embodiment, the TRX1 antibody comprises the heavy chain sequence shown in FIGS. 7A-7C (SEQ ID NO:71 and 72). In another embodiment, the TRX1 antibody comprises the heavy chain sequence shown in FIGS. 7A-7C absent the leader sequence. In still another embodiment, the TRX1 antibody comprises the light chain sequence shown in FIGS. 8A-8B (SEQ ID NO:73 and 74). In still another embodiment, the TRX1 antibody comprises the light chain sequence shown in FIGS. 8A-8B absent the leader sequence.

In one embodiment, the invention provides an anti-CD4 antibody with a light chain variable region (LCVR) having at least one CDR domain derived from a mouse monoclonal antibody, e.g., NSM4.7.2.4. In another embodiment, a light chain variable region (LCVR) has at least one CDR domain comprising an amino acid sequence selected from the group consisting of amino acid residues 44-58, 74-80, and 113-121 of, for example, SEQ ID No.:1 or SEQ ID No.:3 or amino acid residues 24-32, 54-60, and 93-101 of SEQ ID No.:4. In another embodiment, a light chain variable region (LCVR) has at least two CDR domains comprising an amino acid sequence selected from the group consisting of amino acid residues 44-58, 74-80, and 113-121 of, for example, SEQ ID No.:1 or SEQ ID No.:3 or amino acid residues 24-32, 54-60, and 93-101 of SEQ ID No.:4. In yet another embodiment, a light chain variable region (LCVR) has CDR domains comprising the amino acid sequences consisting of amino acid residues 44-58, 74-80, and 113-121 of, for example, SEQ ID No.:1 or SEQ ID No.:3 or amino acid residues 24-32, 54-60, and 93-101 of SEQ ID No.:4.

In one embodiment of the invention, the anti-CD4 antibody comprises a human framework region and a variable region comprising at least one CDR derived from a mouse monoclonal antibody, e.g., NSM4.7.2.4. For example, in one embodiment, an anti-CD4 antibody for use in the methods of the invention comprises at least one light chain CDR sequence selected from the group consisting of amino acid residues 44-58, 74-80, and 113-121 of, for example, SEQ ID No.:1 or SEQ ID No.:3 or amino acid residues 24-32, 54-60, and 93-101 of SEQ ID No.:4. In one embodiment, an antibody for use in the methods of the invention comprises at least two of the light chain CDR sequences. In yet another embodiment, an antibody for use in the methods of the invention comprises at least three of the light chain CDR sequences.

In another embodiment, an anti-CD4 antibody for use in the methods of the invention comprises at least one heavy chain CDR sequence selected from the group consisting of amino acid residues 50-54, 69-85, and 118-126 of, for example, SEQ ID No.:5 or SEQ ID No.:7 or amino acid residues 31-35, 50-66, and 99-107 of SEQ ID No.:8. In one embodiment, an antibody for use in the methods of the invention comprises at least two of the heavy chain CDR sequences. In yet another embodiment, an antibody for use in the methods of the invention comprises at least three of the heavy chain CDR sequences.

Appropriate methods of preparing TRX1 humanized antibody or other anti-CD4 antibody suitable for the purposes of the present invention should be apparent to those skilled in the art from the teachings herein. Such antibody may be prepared by recombinant techniques known to those skilled in the art.

This invention is further illustrated by the following examples, which should not be construed as limiting. The contents of all references, patents and published patent applications cited throughout this application, as well as the Figures, are incorporated herein by reference.

### EXAMPLES

The invention now will be described with respect to the following examples; however, the scope of the present invention is not intended to be limited thereby.

### Example 1

### Construction of TRX1 Antibody Starting from Amino Acid Sequence

A cDNA library was constructed from the mouse hybridoma NSM 4.7.2.4 using the Superscript plasmid system (Gibco/BRL, cat. no. 82485A) according to the manufacturer's suggested protocol. Heavy and light chain cDNAs were cloned from the library by DNA hybridization using as probes rat heavy and light chain gene cDNAs from the rat hybridoma YTS 177.

The rat heavy and light chain gene cDNAs of YTS 177 were isolated from the expression vector pHA Pr-1 as BamH1/Sal 1 fragments and labeled with ³²P and used independently to screen the NSM 4.7.2.4. cDNA library using standard molecular biology techniques (Sambrook, et al., Molecular Cloning, A. Laboratory Manual, 3rd edition, Cold Spring Harbor Laboratory Press, Cold Spring Harbor, N.Y. (2001); Ausubel, et al., Current Protocols in Molecular Biology, John Wiley & Sons, New York (2001).) Sequence analysis of the cDNAs derived from the NSM 4.7.2.4 cDNA library confirmed the NSM 4.7.2.4 heavy chain to be mouse gamma-1 subclass and the NSM 4.7.2.4 light chain to be kappa. The NSM 4.7.2.4 heavy and light V regions (VH and VL, respectively) were reshaped to the human VH and VL regions with the “best fit” or highest sequence similarity in the framework regions to that of the mouse. For the light chain, human antibody HSIGKAW (from EMBL) with a sequence similarity of 79% was used (L A Spatz et al., 1990 J. Immunol. 144:2821-8). The sequence of HSIGKAW VL (SEQ ID No.35) is:

MVLQTQVFISLLLWISGAYGDIVMTQSPDSLAVSLGERATINCKSSQSLL
YSSNNKNYLAWYQQKPGQPPKLLIYWASTRESGVPDRFSGSGSGTDFTLT
ISSLQAEDVAVYYCQQYYSTPPMFGQGTKVEIKRT

D start of framework 1
Q changed to G

For the heavy chain, human antibody A32483 (From GenBank) with a sequence similarity of 74% was used (Larrick, et al., Biochem. Biophys. Res. Comm., Vol. 160, pgs. 1250-1256 (1989)). The sequence of A32483 VH (SEQ ID No.36) is:

LLAVAPGAHSQVQLVQSGAEVKKPGASVKVSCKASGYTFTNYYMHWVRQA
PGQGLEWMGIINPSGNSTNYAQKFQGRVTMTRDTSTSTVYMELSSLRSED
TAVYYCAREKLATTIFGVLIITGMDYWGQGTLVTVSSGSAS

Q start of framework 1

For the humanization process, anti-CD4 light chain clone 77.53.1.2 (insert size 1 kb) and anti-CD4 heavy chain clone 58.59.1 (insert size 1.7 kb) were chosen from the cDNA library and inserts isolated from the pSport vector as Sal I/Not I fragments and cloned into M13mp18 vector to produce single stranded DNA for sequencing and template for mutagenesis. The humanization of NSM 4.7.2.4 was performed by site-directed mutagenesis of the mouse cDNA using a kit from Amersham International (RPN 1523) according to the manufacturer's suggested protocol.

Mutagenesis of the VL gene framework regions was performed using five oligonucleotides ranging in length from 29 to 76 bases. The oligos used were:

Primer #1998
76 bases

(SEQ ID No.37)

5′-TGA CAT TGT GAT GAG CCA ATC TCC AGA TTC TTT GGC
TGT GTC TCT AGG TGA GAG GGC CAC CAT CAA CTG CAA
GGC C
Primer #1999
29 bases

(SEQ ID No.38)

5′-TGA ACT GGT ATC AAC AGA AAC CAG GAG AG
Primer #2000
28 bases

(SEQ ID No.39)

5′-AGA GTC TGG GGT CCC AGA CAG GTT TAG T
Primer #2001
42 bases

(SEQ ID No.40)

5′-GTC TTC AGG ACC CTC CGA CGT TCG GTG GAG GTA CCA
AGC TGG
Primer #2008
52 bases

(SEQ ID No.41)

5′-CAC CCT CAC CAT GAG TTC TCT GCA GGC GGA GGA TGT
TGC AGT CTA TTA GTG T

The oligos were phosphorylated and mutagenesis performed in three steps using no more than two oligos per step to introduce changes according to the following procedure:
(1) Annealing phosphorylated mutant oligos to ssDNA template
(2) Polymerization
(3) Filtration to remove single-stranded DNA
(4) Nicking non mutant strand with Nci I
(5) Digestion of non-mutant strand with Exo III
(6) Repolymerization of gapped DNA
(7) Transformation of competent JM101
(8) Sequencing of clones

Mutations were confirmed by single strand DNA sequencing using M13 primers −20 and −40 and also the mutagenic primers # 1999 and # 2000.

A Sal I site at the 5′ end of the variable region was changed to Hind III by linker oligos #2334 and #2335 to allow cloning of the variable region as a Hind III/Kpn I fragment into the light chain constant region of CAMPATH-1H.

Primer #2334
24 bases

(SEQ ID No.42)

5′-AGC TTT ACA GTT ACT GAG CAC ACA
Primer #2335
24 bases

(SEQ ID No.43)

5′-TCG ATG TGT GCT CAG TAA CTG TAA

Mutagenesis of the VH gene framework regions was performed using five oligonucleotides ranging in length from 24 to 75 bases. The oligos used were:

Primer #2003
75 bases

(SEQ ID No.44)

5′-GGT TCA GCT GGT GCA GTC TGG AGC TGA AGT GAA
GAA GCC TGG GGC TTC AGT GAA GGT GTC CTG TAA GGC
TTC TGG
Primer #2004
52 bases

(SEQ ID No.45)

5′-AGC TGG GTG AGG CAG GCA CCT GGA CAG GGC CTT
GAG TGG ATG GGA GAG ATT T
Primer #2005
60 bases

(SEQ ID No.46)

5′-CAA GGG CAG GGT CAC AAT GAC TAG AGA CAC ATC
CAC CAG CAC AGT CTA CAT GGA ACT CAG
Primer #2006
43 bases

(SEQ ID No.47)

5′-CAG CCT GAG GTC TGA GGA CAC TGC GGT CTA TTA CTG
TGC AAG A
Primer #2007
24 bases

(SEQ ID No.48)

5′-GCC AAG GGA CACG TAG TCA CTG TGT

Mutagenesis was carried out as described above for the light chain again using no more than two oligos at a time to introduce the changes. Mutations were confirmed by single strand DNA sequencing using M13 primers −20 and −40 as well as the mutagenic primers #2002 and #2004.

Primer #2002 was used to correct a reading frame error in starting clone 58.59.1.

Primer #2002
39 bases

(SEQ ID No.49)

5′-ACT CTA ACC ATG GAA TGG ATC TGG ATC TTT CTC CTC
ATC

Primer #2380 was used to correct extra mutation added by #2004 which was missed in the first sequencing.

Primer #2380
39 bases

(SEQ ID No.50)

5′-TCA CTG CCT ATG TTA TAA GCT GGG TGA GGC AGG CAC
CTG

As with the light chain, the heavy chain 5′ Sal I site was changed to Hind III using linker oligo's #2334 and #2335 to allow cloning of the heavy chain variable region as Hind III/Spe I (site introduced by primer #2007) fragment into the heavy chain constant region of CAMPATH-1H.

Construction of Heavy Chain

The following samples of DNA were used:

1. Plasmid 1990. Human gamma-1 heavy chain constant region gene cloned into pUC18 (obtained from Martin Sims, Wellcome Foundation Ltd).

2. Plasmid 2387: Reshaped heavy chain of NSM 4.7.2.4 containing human framework regions and mouse gamma 1 constant region.

A Sal I site in the reshaped CD4 heavy chain was altered to a Hind III site. The variable region gene was excised by digestion with Hind III/Spe I and ligated with the constant region gene in plasmid 1990 to give a complete humanized heavy chain (plasmid 2486). The heavy chain gene was cut out of this plasmid with Hind III/EcoR I and ligated with the expression vector pEE6.

Construction of Light Chain

The following samples of DNA were used.

1. Plasmid 2028; CAMPATH-1H light chain gene cloned into M13mp18 at Sal I/BamH I restriction site.

2. Plasmid 2197; Reshaped light chain of NSM 4.7.2.4 containing human framework regions and mouse kappa constant region. A Kpn I site already had been introduced between variable and constant portions of this gene.

A Kpn I restriction site was introduced into the CAMPATH 1H light chain gene corresponding to the site in plasmid 2197 and an EcoR I site was introduced at the 3′ end of the constant region. The constant region gene was excised from this plasmid (2502) by digestion with Hind III/Kpn I.

Meanwhile a Sal I site in plasmid 2197 was changed to a Hind III site (this step had to be repeated because a frame-shift mutation inadvertently was introduced the first time). The new plasmid (2736) was digested with Hind III/Kpn I. The CD4 variable region fragment was cloned into a plasmid containing the kappa constant region gene from plasmid 2502 to give a complete humanized light chain (plasmid 2548). The light chain gene was cut out from this plasmid with Hind III/EcoR I and ligated with the expression vector pEE12 to give plasmid 2798.

Ligation of Heavy and Light Chains and Expression in NSO Cells

The heavy chain gene was excised from the pEE6 vector by digestion with Sal I/Bgl H and cloned into the light chain pEE12 vector which had been digested with BamH I/Sal I.

The final vector construct was checked by restriction digests with Hind III, EcoR I, Sal I, BamH I, BgI II and Spe I for the presence of the expected fragments, including 700 bp light chain, 1400 bp heavy chain, 2300 bp fragment of pEE6 and 7000 bp fragment of pEE12.

The pEE12 vector was linearized by digestion with Sal I and transferred into NSO cells by electroporation, following a standard protocol (Celltech 1991) except that the selection medium was slightly modified, being based on IMDM rather than DMEM. Transfectants were selected in medium lacking glutamine, supplemented with dialysed FCS, ribonucleosides, glutamic acid, and asparagine as recommended.

The transfection mixes were cultured in three 96-well plates, and of 36 growing wells which were tested, 5 were strongly positive for production of human heavy and light chains (18 others were positive for one or other, or weakly positive for both).

A clone, designated SDG/B7B.A.7 was selected and stored frozen but no further characterization has been done on this wild type antibody.

Construction of Mutant IgG1 Antibody Designated to Abolish Effector Functions

Due to concerns about side effects of other CD4 antibodies reported in various clinical trials, it is considered desirable to avoid the possibility of engaging Fc receptors. Human IgG4 is thought to have minimal Fc binding or complement-activating ability. However, experiments have show that it does engage Fc receptors in some individuals (Greenwood et al., Eur. J. Immunol., Vol. 23, pgs. 1098-1104, 1993), and clinical studies with a human IgG4 variant to CAMPATH-1H have demonstrated an ability to kill cells in vivo (Isaacs et al., Clin. Exp. Immunol. Vol. 106, pgs. 427-433 (1996)). To eliminate the possibility of binding Fc receptors, constructs were made with mutations in the IgG1 heavy chain constant region.

TRX1 can be made to have mutations, e.g., Leu²³⁶ to Ala and Gly²³⁸ to Ala, as shown in SEQ ID Nos.:5 and 6, and SEQ ID Nos.:21 and 22. These particular residues were chosen because they are predicted to disrupt maximally binding to all three types of human Fc receptors for IgG. Either mutation is sufficient to reduce binding to Fc(RI (Woof, et al., Mol. Immunol, Vol. 332, pgs. 563-564, 1986; Duncan, et al., Nature, Vol. 332, pgs. 563-564 1988; Lund, et al., J. Immunol Vol. 147, pgs. 2657-2662 1991) or Fc(RII (Lund et al., 1991; Sarmay et al., Mol. Immunol., Vol. 29, pgs. 633-639 1992) whereas Gly²³⁸ to Ala has the biggest effect on binding to Fc(RIII (Sarmay et al., 1992).

The following samples of DNA were used.

1. Plasmid 2555 and Plasmid 2555 Mut.; the humanized V_{H} region of NSM 4.7.2.4 cloned into pEE6 expression vector at a Hind III/Spe I restriction site. Plasmid 2555 then was mutated by site directed mutagenesis such that amino acid residue Asn¹⁰¹ is changed to Asp¹⁰¹, as shown in SEQ ID Nos.:5 and 6, and SEQ ID Nos.:21 and 22. The resulting plasmid was plasmid 2555 Mut.

2. Plasmid 2798; the humanized V_{H} region of NSM 4.7.2.4 was joined to human kappa constant regions to give approx 700 bp fragment cloned into pEE12 expression vector at a Hind III/EcoR I.

3. Plasmid MF4260; the human IgG1 heavy chain was associated with the humanized CD 18 V_{H} region, having the mutations Leu²³⁶ to Ala and Gly²³⁸ to Ala as well as a Spe I restriction site introduced into framework region 4, cloned into pUC18.

The purpose of the Spe I restriction site is to allow separation and recombination of different variable regions.

The CD18 V_{H} region gene is excised from plasmid MF 4260 by digestion with Spe I and Hind III and the remaining vector, now having only the relevant heavy chain constant region, was purified using Geneclean. It is ligated with the humanized V_{H} region DNA of NSM 4.7.2.4 which has been isolated from plasmid 2555 Mut in the same way. The product is used to transform “Sure” cells and colonies are checked for the presence of the expected 1400 bp complete heavy chain insert.

The complete V_{H} and constant region insert was excised from the pUC vector by digestion with Hind III and EcoR I. The 1400 bp fragment is purified using QiaexII (Qiagen) and then ligated in turn into the vector pEE6, which has previously been cut with the same enzymes.

The next step was to excise the CD4 heavy chain genes from the pEE6 vector and clone them into pEE12, already containing the humanized CD4 light chain gene (plasmid 2798). The pEE6 vector was digested with Sal I and BgI II and the pEE12 vector is digested with Sal I and BamH I to create the appropriate sites for re-ligation.

The final vector construct was checked by restriction digests with Hind III, EcoR I, Sal I and Spe I for the presence of the expected fragment, i.e., 700 bp light chain, 1400 bp heavy chain, 2300 bp fragment of pEE6, and 7000 bp fragment of pEE12.

The pEE12 vector was linearized by digestion with Sal I and transfected into NSO cells by electroporation as above. The transfection mixes were cultured in six 96-well plates, and of 90 growing wells which were tested, all were positive for production of human heavy and light chains. At this stage a sample of the pEE12 vector DNA was digested with Sal I and precipitated with ethanol.

Transfection and Selection of Final Transfectant

The TRX1 expression vector DNA ‘pTX/C4’ was transfected into exponentially growing CHO/dhfr− cells that were expanded from the ‘Parental CHO DHFR-MCB1’.

The TRX1 DNA was linearized, and (10) μg of the linearized TRX1 DNA was added to 1 ml (3×10⁶ cells) of exponentially growing CHO/dhfr− cells in an electroporation cuvette on ice. The cells were transfected (using a BioRad Gene Pulser II) set to 1000 volts, capacitance of 25 microfarads, and resistance of ∞ ohms. After electroporation, the cells were placed on ice for 10 minutes followed by addition to a T25 tissue culture flask and incubation in a 37° C., 5% CO₂ incubator. Transformants were selected for the phenotype of neomycin resistance followed by selection and amplification of DHFR+ transformants using α-MEM supplemented with methotrexate, 10% US sourced (harvested in 2001), irradiated, dialyzed FBS, and neomycin. Cells that survived in the culture medium containing methotrexate and neomycin were screened for productivity and cloned by limiting dilution in 96-well plates. These clones were then screened for high producers. Subclone ‘E9/3A2’ was found to have the highest specific productivity. This clone was selected and subsequently expanded for preparation of a pre-seed stock.

Purification of the Antibody

Culture supernatant is purified by using a Biopilot chromatography system (Pharmacia) in three steps as follows:
(1) Affinity chromatography on a column of Protein A-Sepharose Fast Flow
(2) Ion exchange chromatography on S-Sepharose Fast Flow
(3) Size exclusion chromatography on Superdex 20.

The purified product was filtered and pooled into a single biocontainer.

Throughout the purification process, precautions are taken to ensure that the system remains aseptic. All buffers and reagents are passed through a 0.2 micron membrane filter and the purified product is also passed through a 0.2 micron filter before being pooled. After a batch of antibody has been processed, the entire chromatography system and columns are sanitized with 0.5M NaOH, washed with sterile PBS and stored in 20% ethanol. Before it is used again, the ethanol is washed out with sterile PBS and a complete trial run is carried out. Samples of buffers and column eluates are checked for endotoxin level.

### Example 2

### Construction of TRX1 Antibody Starting from Nucleotide Sequence

Cloning of Human Constant Regions

Heavy Chain Constant Region

The human gamma 1 heavy chain constant region (IgG1) is amplified from human leukocyte cDNA (QUICK-Clone™ cDNA Cat. No. 7182-1, Clontech) using the following primer set and cloned into pCR-Script (Stratagene). The plasmid containing the human gamma 1 heavy chain constant region in pCR-Script is designated pHCγ-1.

primer hcγ-1

(SEQ ID No.51)

Spe I
5′ primer: 5′- ACT AGT CAC AGT CTC CTC AGC
primer hcγ-2

(SEQ ID No.52)

EcoR I
3′ primer: 5′- GAA TTC ATT TAC CCG GAG ACA G

Non-Fc binding mutations (Leu²³⁶ Ala, Gly³⁸ Ala) are made in the heavy chain constant region by site-directed mutagenesis using the following primer and the Transformer™ Site-Directed Mutagenesis Kit from Clontech (Cat. No. K1600-1). The plasmid containing the human gamma 1 heavy chain non-Fc binding mutant constant region in pCR-Script is designated pHCγ-1Fcmut.

primer hcγ-3

(SEQ ID No.53)

Fc mut oligo: 5′- CCG TGC CCA GCA CCT GAA CTC
GCG GGG GCA CCG TCA GTC TTC CTC CCC C

Light Chain Constant Region

The human kappa light chain constant region is amplified from human leukocyte cDNA (QUICK-Clone™ cDNA Cat. No. 7182-1, Clontech) using the following primer set and cloned into pCR-Script (Stratagene). The plasmid containing the human kappa light chain constant region in pCR-Script is designated pLCK-1.

primer lcκ-1

(SEQ ID No.54)

Kpn I
5′ primer: 5′- GGT ACC AAG GTG GAA ATC AAA CGA AC
primer lcκ-2

(SEQ ID No.55)

Hind III
3′ primer: 5′- AAG CTT CTA ACA CTC TCC CCT GTT G

Synthesis, Construction and Cloning of TRX1 Variable Regions

The heavy and light chain variable regions are constructed from a set of partially overlapping and complementary synthetic oligonucleotides encompassing the entire variable regions. The oligonucleotide set used for each variable region is shown below.

Heavy Chain Variable Region Synthetic Oligonucleotides

Coding Strand Heavy Chain Variable Region Primers

primer hv-1 (1 - 72) + 6 nucleotide linker

(SEQ ID No.56)

5′- aagctt ATG GAA TGG ATC TGG ATC TTT CTC CTC ATC
CTG TCA GGA ACT CGA GGT GTC CAG TCC CAG GTT CAG
CTG GTG
primer hv-2 (120 - 193)

(SEQ ID No.57)

5′- C TGT AAG GCT TCT GGA TAC ACA TTC ACT GCC TAT
GTT ATA AGC TGG GTG AGG CAG GCA CCT GGA CAG GGC
CTT G
primer hv-3 (223 - 292)

(SEQ ID No.58)

5′- GGT AGT AGT TAT TAT AAT GAG AAG TTC AAG GGC
AGG GTC ACA ATG ACT AGA GAC ACA TCC ACC AGC ACA G
primer hv-4 (322 - 399)

(SEQ ID No.59)

5′- GAG GAC ACT GCG GTC TAT TAC TGT GCA AGA TCC
GGG GAC GGC AGT CGG TTT GTT TAC TGG GGC CAA GGG
ACA CTA GT
Non-Coding Strand Heaiy Chain Variable Region
Primers
primer hv-S (140 - 51)

(SEQ ID No.60)

5′- GTG TAT CCA GAA GCC TTA CAG GAC ACC TTC ACT
GAA GCC CCA GCC TTC TTC ACT TCA GCT CCA GAC TGC
ACC AGC TGA ACC TGG GAC TGG
primer hv-6 (246 - 170)

(SEQ ID No.61)

5′- CTT CTC ATT ATA ATA ACT ACT ACC GCT TCC AGG
ATA AAT CTC TCC CAT CCA CTC AAG GCC CTG TCC AGG
TGC CTG CC
primer hv-7 (342 - 272)

(SEQ ID No.62)

5′- GTA ATA GAG CGC AGT GTC CTC AGA CCT GAG GCT
GCT GAG TTG CAT GTA GAC TGT GCT GGT GGA TGT GTC TC

Light Chain Variable Region Synthetic Oligonucleotides

Coding Strand Light Chain Variable Region Primers

primer lv-1 (1 - 63) + 6 nucleotide linker

(SEQ ID No.63)

5′- gaattc ATG GAG ACA GAC ACA ATC CTG CTA TGG GTG
CTG CTG CTC TGG GTT CCA GGC TCC ACT GGT GAC
primer lv-2 (93 - 158)

(SEQ ID No.64)

5′- GGC TGT GTC TCT AGG TGA GAG GGC CAC CAT CAA
CTG CAA GGC CAG CCA AAG TGT TGA TTA TGA TGG
primer lv-3 (184 - 248)

(SEQ ID No.65)

5′- CAG AAA CCA GGA CAG CCA CCC AAA CTC CTC ATC
TAT GTT GCA TCC AAT CTA GAG TCT GGG GTC CC
primer lv-4 (275 - 340)

(SEQ ID No.66)

5′- GGA CAG ACT TCA CCC TCA CCA TCA GTT CTC TGC
AGG CGG AGG ATG TTG CAG TCT ATT ACT GTC AGC
Non-Coding Strand Light Chain Variable Region
Primers
primer lv-5 (109 - 43)

(SEQ ID No.67)

5′- CAC CTA GAG ACA CAG CCA AAG AAT CTG GAG ATT
GGG TCA TCA CAA TGT CAC CAG TGG AGC CTG GAA C
primer lv-6 (203 - 138)

(SEQ ID No.68)

5′- GGT GGC TGT CCT GGT TTC TGT TGA TAC CAG TTC
ATA TAA CTA TCA CCA TCA TAA TCA ACA CTT TGG
primer lv-7 (294 - 228)

(SEQ ID No.69)

5′- GGT GAG GGT GAA GTC TGT CCC AGA CCC ACT GCC
ACT AAA CCT GTC TGG GAC CCC AGA CTC TAG ATT G
primer lv-8 (378 - 319)

(SEQ ID No.70)

5′- GGT ACC TCC ACC GAA CGT CGG AGG GTC CTG AAG
ACT TTG CTG ACA GTA ATA GAC TGC AAC

After HPLC purification and removal of organic solvents the oligonucleotides are resuspended in TE pH8.0 and phosphorylated. An aliquot of each oligonucleotide in the respective variable region set then are combined in equal molar amounts. The oligonucleotide mixtures are heated to 68° C. for 10 minutes and allowed to cool slowly to room temperature. The annealed oligonucleotides then are extended to produce double stranded variable region DNA fragments. For the extension, dNTPs are added to a final concentration of 0.25 mM followed by an appropriate volume of 5×T4 DNA polymerase buffer [165 mM Tris acetate, pH 7.9, 330 mM sodium acetate, 50 mM magnesium acetate, 500 (g/ml BSA, 2.5 mM DTT] and 4 units of T4 DNA polymerase. The mixture is incubated at 37° C. for 1 hour followed by heat inactivation of the T4 DNA polymerase at 65° C. for 5 minutes.

The double stranded DNA is ethanol precipitated and resuspended in the same volume of TE pH 8.0. An appropriate volume of 5×T4 DNA ligase buffer [250 mM Tris-HCl, pH7.6, 50 mM MgCl₂, 5 mM ATP, 5 mM DTT, 25% w/v polyethylene glycol-8000] then is added to the double stranded DNA followed by 2 units of T4 DNA ligase and the mixture incubated for 1 hour at 37° C. to ligate the extended fragments. The T4 DNA ligase then is heat inactivated at 65° C. for 10 minutes. The variable region DNA fragments then are phenol extracted, ethanol precipitated, and resuspended in TE, pH 8.0 and cloned into pCR-Script (Stratagene). The resulting plasmid containing the heavy chain variable region is designated pHv-1 and the plasmid containing the light chain variable region was designated pLV-1.

The final heavy and light chain expression vectors are constructed in pcDNA 3.1 (Invitrogen). For the heavy chain expression vector, the Fc mutated constant region is released from plasmid pHC-1Fcmut by digestion with Spe I and EcoR I and isolated by agarose gel electrophoresis. The heavy chain variable region is released from plasmid pHV-1 by digestion with Hind III and Spe I and isolated by agarose gel electrophoresis. The two fragments in equal molar amounts are ligated into the Hind III/EcoR I sites of pcDNA3.1(+) (Invitrogen) using standard molecular biology techniques. The resulting TRX1 heavy chain expression vector is designated pTRX1/HC.

Similarly, for the light chain expression vector, the light chain constant region is released from plasmid pLC-1 by digestion with Kpn I and Hind III followed by agarose gel purification. The light chain variable region is released from pLV-1 by digestion with EcoR I and Kpn I followed by agarose gel purification. The two light chain fragments in equal molar amounts are ligated into the EcoR I/Hind III sites of pcDNA3.1(−) (Invitrogen) using standard molecular biology techniques yielding the TRX1 light chain expression vector pTRX1/LC.

For production of TRX1 antibody, the TRX1 heavy chain and TRX1 light chain expression plasmids are cotransfected into CHO cells using standard molecular biology techniques.

### Example 3

### Construction of Aglycosylated TRX1 Antibody

A humanized antibody, e.g., the components of the humanized antibody, e.g., light chain and heavy chain, each containing constant regions and variable regions, e.g., amino acid sequences are shown in Seq ID Nos.: 9, 11, 12, 13, 15, and 16 (FIGS. 2A, 2C, 2D, 2E, and 2G), and were produced by a procedure similar to that of Example 1. The humanized antibody is an aglycosylated antibody.

### Example 4

### Construction of Aglycosylated TRX1 Antibody

A humanized antibody, e.g., the components of the humanized antibody, e.g., light chain and heavy chain, each containing constant regions and variable regions, e.g., amino acid sequences are shown in Seq ID Nos.: 25, 27, 28, 29, 31, and 32 (FIGS. 4A, 4C, 4D, 4E, and 4G), and is produced by a procedure similar to that of Example 1. The humanized antibody is an aglycosylated antibody.

### Example 5

### Treatment of a Primate with TRX1 Antibody

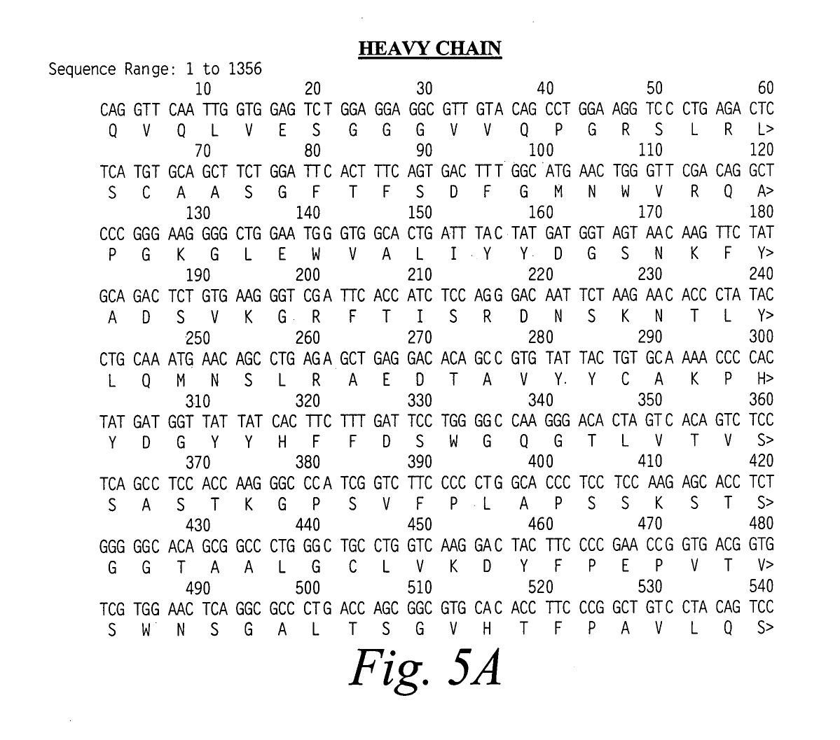

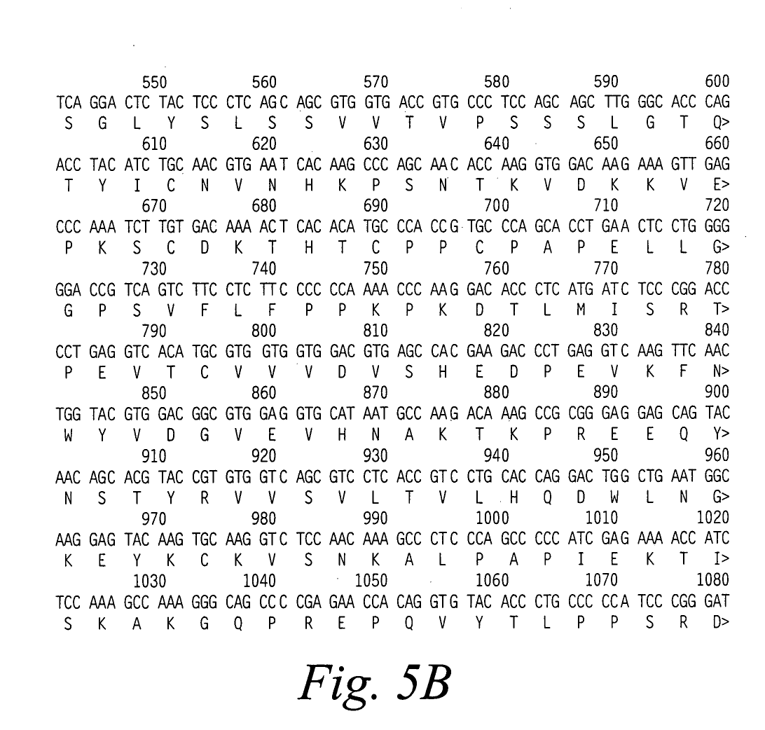

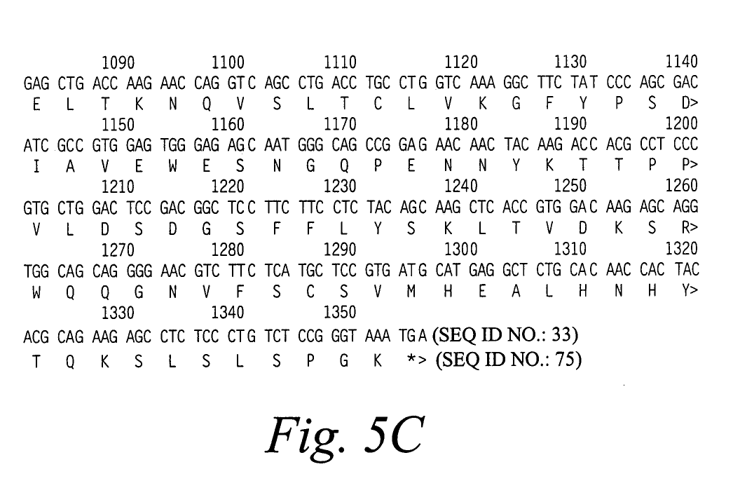

A baboon having a weight of 4.6 kg received a mismatched kidney transplant from another baboon on day 1 and was treated with both the CD4 antibody, e.g., the humanized antibody, e.g., the components of the humanized antibody, e.g., light chain and heavy chain, each containing a constant region and a variable region, e.g., amino acid sequences shown in Seq ID Nos.: 9, 11, 12, 13, 15, and 16, and with a depleting humanized CD8 antibody, the amino acid sequences of which is shown in SEQ ID Nos.:33 (FIGS. 5A-5C) and 34 (FIG. 6) (nucleic acid sequences are set forth as SEQ ID Nos.:75 (FIGS. 5A-5C) and 76 (FIG. 6) in accordance with the following Protocol of Table 1.

The animal has survived for more than 80 days without receiving an immunosuppressant. In addition except for a period of about two days, creatinine levels were below 2 mg/dL.

TABLE 1
Protocol Study 2

| | DAYS | | | | | | | | | | | | | | | |
|---|---|---|---|---|---|---|---|---|---|---|---|---|---|---|---|---|
| ACTION | 0 | 1 | 2 | 3 | 4 | 5 | 6 | 7 | 8 | 9 | 10 | 11 | 12 | 13 | 14 | 15 |
| Treatments | | | | | | | | | | | | | | | | |
| Renal transplantation | | X | | | | | | | | | | | | | | |
| CD4 antibody (iv)² | X | | X | | X | | | X | | X | | X | | | | |
| CD8 antibody (iv)³ | X | | X | | X | | | X | | X | | X | | | | |

²CD4 antibody 40 mg/kg on day 0 and 20 mg/kg for all other doses was given by iv infusion over 1 hour
³CD8 antibody 6 mg/kg given as an iv bolus after the CD4 antibody infusion

These materials and methods were used in the following examples:

Equine Immunoglobulin as a Source of Antigen.

Antivenin (Crotalidae polyvalent) was purchased from Fort Dodge Laboratories (Overland Park, Kans.) and reconstituted with diluent provided by the manufacturer and used as our source of equine Ig. The solution was passed through a 2 micron syringe filter and aggregated by diluting to 25 mg/ml in 0.9% saline and incubating at 64° C. for 35 min followed by overnight incubation on ice. The material was stored at −80° C. until use. The amount of aggregated material in each lot was determined by HPLC size exclusion chromatography and ranged from 21.2% to 29.9% of total protein.

TRX1 Production and Purification

TRX1 is derived from the mouse anti-human CD4 hybridoma, NSM 4.7.2.4. The parental heavy and light chain cDNA were cloned from an NSM 4.7.2.4 cDNA library by cross hybridization with rat heavy and light chain gene cDNA probes using standard molecular biology techniques. Sequence analysis of the cDNA derived from NSM 4.7.2.4 confirmed the heavy chain isotype to be gamma-1 and the light chain kappa. The NSM 4.7.2.4 mouse VH and VL regions were reshaped to human VH and VL regions using “best fit” or human frameworks with the highest sequence similarity to that of the mouse VH and VL. For the light chain, human antibody HSIGKAW (from EMBL) with a sequence similarity of 79% was used as the target sequence. For the heavy chain, human antibody A32483 (GenBank) with a sequence similarity of 74% was used. The humanization was performed by site-directed mutagenesis of the mouse cDNA clones. To eliminate antibody binding to Fc receptors as well as complement fixation, a single amino acid substitution was introduced in the Fc region at amino acid position 297 of γ1 heavy chain constant region by site-directed mutagenesis eliminating the site of N-linked glycosylation.

TRX1 antibody was produced at the Therapeutic Antibody Centre (Oxford, UK) by hollow fiber fermentation of CHO cell transfectants. The antibody was purified from culture supernatant by Protein A affinity chromatography followed by cation/anion exchange, nanofiltration, and finally size exclusion chromatography. The purified material was formulated in PBS and stored at −80° C.

Tolerance Induction and Challenge Protocol

All baboon work was performed at the Southwest Foundation for Biomedical Research (San Antonio, Tex.) under a protocol approved by the Institutional Animal Care and Use Committee. Seven to twenty-one days prior to study, animals were screened by physical examination, CBC and serum chemistries. Lymphocyte subset numbers and CD4 expression level on CD3⁺ cells were determined for baseline values. A second set of baseline values was collected on day −1 just prior to the first TRX1 or saline infusion. Animals were sedated with a single dose of 10 mg/kg ketamine plus 5 mg diazepam as needed to facilitate handling. TRX1 and saline infusions were administered i.v. at a rate of 30 ml/hr. Temperature, blood pressure and respiration were monitored during and after infusions. Animals were examined for skin rashes and lymphadenopathy at the time of each infusion and at the time of subsequent serum sample collections. In addition animals were monitored daily for signs of discomfort, malaise, arthralgia and gastrointestinal complications. The first dose of antigen (equine Ig) was given on Day 0 as a 10 mg/kg i.v. bolus. All subsequent doses of equine Ig (Days 4, 8, 68, 95 and 130) were given as a 10 mg/kg s.c. bolus, except for the last challenge on Day 130, which was a 1 mg/kg s.c. bolus.

Animals were immunized with SRBC (HemoStat Laboratories, Dixon, Calif.) to demonstrate immunocompetence to a neo-antigen after TRX1 exposure. All animals received a single i.v. injection of a 10% SRBC solution in 0.9% sterile saline at a dose of 1.7 ml/kg on Day 68 of the study.

TRX1 Serum Concentration

The concentration of TRX1 in serum was determined by ELISA. 50 μl of a 5 μg/ml solution of soluble CD4 in PBS (kindly provided by the Therapeutic Antibody Centre, Oxford, UK) was dispensed into 96-well plates and incubated overnight at 2-8° C. After three washes with PBS containing 0.05% Tween 20 (Wash Buffer) plates were blocked with 1% BSA, 0.05% Tween 20 in PBS (Blocking Buffer) for 1 hr at 37° C. and stored at 2-8° C. Immediately prior to use plates were washed three times with Wash Buffer. Baboon serum samples were prepared from a 1:10 or 1:100 starting dilution in Blocking Buffer followed by serial 1:10 dilutions and transfer of 50 μl of diluted sample to the soluble CD4 coated plates. A standard curve included on each plate was prepared from a 1 μg/ml solution of unconjugated TRX1 serially diluted 1:4. Following a 2 hr incubation at 37° C., plates were washed three times and 50 μl of a peroxidase-conjugated donkey anti-human IgG (0.08 μg/ml in Blocking Buffer) was added to each well. Plates were incubated for 1 hr at room temperature, washed three times and developed. TRX1 serum concentrations were calculated from all OD values falling within the linear portion of the TRX1 standard curve.

Immune Response to Equine Ig

Baboon anti-globulin responses to equine Ig were determined by ELISA. 96-well plates coated with 50 μl/well of a 10 μg/ml solution of antivenin in carbonate buffer were incubated overnight at 4° C. Plates were then washed three times and blocked for 2 hr at 37° C. Following the blocking step, plates were washed three times and baboon serum samples added to wells (50 μl/well) using a 3-fold serial dilution scheme beginning with a 1:10 dilution and incubated for 2 hr at room temperature.

After three washes peroxidase conjugated rabbit anti-human IgG/IgM antibody (diluted 1/10,000) was added to each well (50 μl/well) and incubated for 1 hr at room temperature. Plates were then washed three times and 100 μl of substrate added to each well followed by incubation at room temperature for 8 min. The assay was standardized by including on each plate a positive control serum. The positive control serum was obtained from a previously immunized animal and was used as a standard in all assays at a dilution of 1:25,000. Titer is defined as the reciprocal of the dilution resulting in an OD value equivalent to twice the OD value of a 1:25,000 dilution of the standard.

SRBC Hemolysis Assay

Immune response to SRBC was assessed by hemolysis. Serum samples were heat inactivated at 56° C. for 30 min followed by preparation of a 2-fold dilution series starting from a 1:10 dilution in PBS plus 0.1% BSA. 100 μl of the diluted serum was combined with an equal volume of a 1% SRBC solution followed by the addition of 100 μl Guinea pig complement (Sigma-Aldrich) pre-adsorbed with SRBC diluted 1:10 in PBS. The plates were incubated at 37° C. for 30 min. Titer is defined as the reciprocal of the highest dilution of serum that did not cause obvious hemolysis.

Antibodies and Flow Cytometry

Normal donkey serum, donkey anti-human IgG-biotin, donkey anti-human IgG F(ab′)₂-biotin, donkey anti-human IgG-peroxidase, donkey IgG-biotin, rabbit anti-human IgG/IgM and human IgG-biotin were purchased from Jackson ImmunoResearch. FITC conjugated mouse anti-human CD4, clone M-T441, and FITC conjugated mouse IgG2b, clone BPC 4, were purchased from Ancell, Inc. Mouse anti-human CD3 FITC, clone SP34, mouse IgG3 FITC, and mouse anti-human CD45RA-PE were purchased from BD Pharmingen. Mouse anti-human CD8-PerCP and mouse IgG1-PerCP were purchased from BD Biosciences. Streptavidin-Quantum Red was purchased from Sigma-Aldrich and FITC and Cy5 conjugated standard beads from Bangs Laboratories (Fishers, Ind.).

CD4 saturation was determined as a function of free CD4 sites on circulating lymphocytes. 100 μl of heparinized whole blood was pelleted by centrifugation and plasma removed by aspiration. Cells were re-suspended in 100 μl of a 1.0 μg/ml solution of biotinylated TRX1 or biotinylated human IgG. Following a 20 min incubation on ice cells were washed with 1 ml of Wash Buffer and incubated with 50 μl Streptavidin Quantum Red (1:5 dilution of stock) for 20 min on ice. RBC were then lysed by the addition of 2 ml of Lysis Buffer (0.15M NH₄Cl, 10 mM KHCO₃, 100 μM disodium EDTA). Samples were vortexed and incubated at room temperature until clear (approximately 10 min). RBC debris was removed by centrifugation and washing with 1 ml Wash Buffer. Cells were fixed by the addition of PBS, 0.1% formalin. Intra-day fluorescence sensitivity variation was controlled by using FITC and Cy5 conjugated standard beads.

CD4⁺ lymphocyte counts

The number of CD4⁺ lymphocytes in peripheral blood was determined by multiplying the absolute lymphocyte count obtained from CBC data by the percentage of CD4⁺ lymphocytes. The percentage of CD4⁺ lymphocytes in whole blood was determined by flow cytometry as the percentage of CD4⁺ cells in the lymphocyte gate staining with FITC-conjugated M-T441, a mouse antibody recognizing domain 2 of CD4 that does not compete with TRX1 binding to CD4. The TRX1 antibody used in these examples was a humanized IgG1 antibody recognizing domain 1 of human CD4 further modified by introducing a single amino acid substitution (Asn to Ala) at position 297 in the heavy chain constant region, so eliminating a major glycoslyation site necessary for high affinity Fc receptor interactions and complement binding (Bolt, S., E. et al. 1993. Eur. J. Immunol. 23:403; Friend, P. J., et al. 1997. Transplantation 68:1632; Routledge, E. G., et al. 1995. Transplantation 60:847).

Study Design

To identify a model species in which to test tolerance induction with TRX1, a number of non-human primates were screened including African green monkey, cynomolgus and rhesus macaque, baboon and chimpanzee, for cross-reactivity with TRX1. While all showed some degree of immunoreactivity, only in chimpanzee and baboon was the binding affinity comparable to human. Thus, baboon was selected as the model species.

As a target antigen for tolerance induction, a simple, yet clinically relevant, model antigen was sought. This allowed testing for antigen specific tolerance as well as optimization of the induction protocol prior to investigation in more complex models such as organ transplant and autoimmune diseases. A well characterized immunogenic biologic antivenin, or anti-venom, Crotalidae polyvalent was selected—a commercial preparation of equine immune serum globulins (equine Ig) isolated from horses immunized with pit viper venoms (Jurkovich, G. J., et al., 1988. J Trauma 28:1032; Dart, R. C., and J. McNally. 2001. Ann. Emerg. Med. 37:181). To ensure immunogenicity of the equine Ig the material was heat-aggregated and the preparation tested in a pilot experiment to determine a dose and route of administration that would generate a robust immune response prior to use in the tolerance induction protocol.

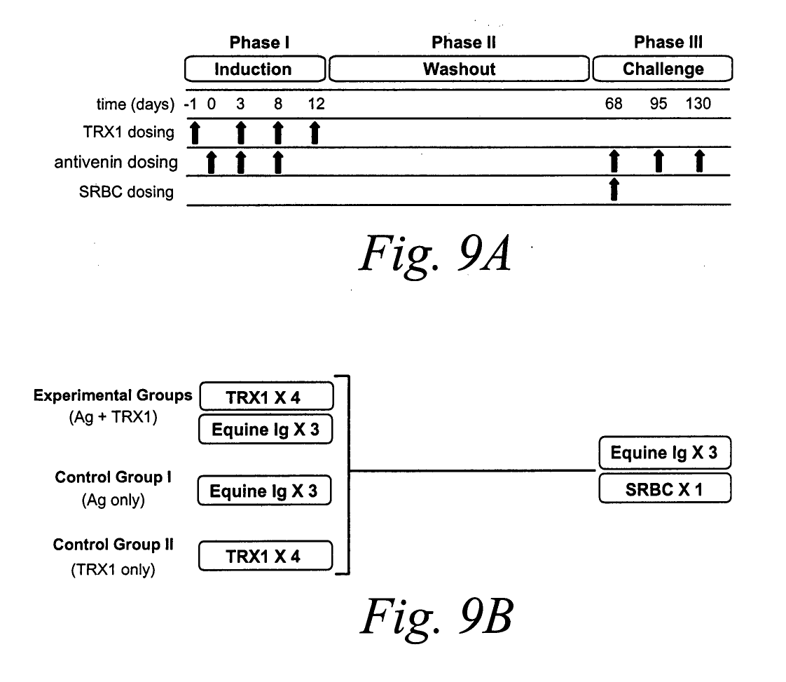

To investigate the feasibility of tolerance induction with TRX1 in baboons an experimental protocol divided into 3 phases—induction, washout, and challenge (FIG. 9A) was designed and implemented by assigning twenty-one baboons (Papio cynocephalus anubis) to one of 7 groups (3 animals/group) including 4 experimental and 3 control groups (FIG. 9B). The experimental arm of the induction phase consisted of 4 TRX1 dosing cohorts of 1, 10, 20, or 40 mg/kg per dose infused 4 times over 13 days on day −1, day 3 or 4, day 8 and day 12. A 10 mg/kg i.v. bolus of heat aggregated antigen (equine Ig) was delivered on day 0 followed on days 4 and 8 with a s.c. bolus of the same dose. In the control arm, animals in control group I (antigen only), were infused with an equivalent volume of normal saline instead of TRX1 at each time point exactly as animals in the experimental groups. Control group II, (TRX1 only), was comprised of 2 cohorts, 20 mg/kg and 40 mg/kg TRX1, dosed on the same schedule as the experimental groups but receiving normal saline instead of equine Ig during the tolerization phase. TRX1 serum concentrations were determined 24 hours after the first dose of antibody and immediately prior to the 3 subsequent doses as well as weekly thereafter. Serum levels of TRX1 and equine Ig were monitored until no longer detectable (washout phase), at which time all animals were challenged by s.c. injection with heat-aggregated equine Ig (challenge phase).

### Example 6

### TRX1 Suppresses the Humoral Response During Induction without Depletion of T-Cells

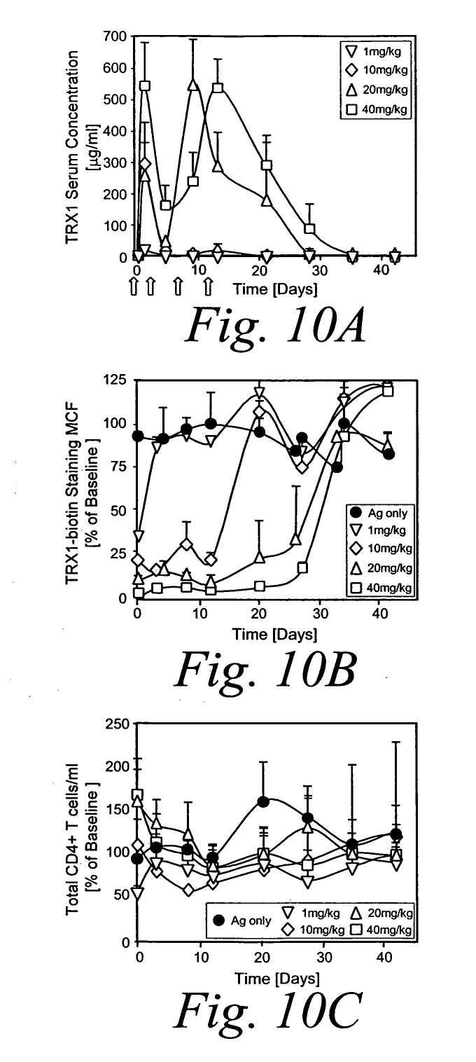

A dose dependent increase in TRX1 serum concentration was evident 24 hours after the first dose ranging from a mean of 15.6±4.1 μg/ml (n=3) in animals receiving 1 mg/kg up to a mean of 542.5±138.1 μg/ml (n=6) in those receiving 40 mg/kg (FIG. 10A). Serum concentrations of TRX1 determined immediately prior to subsequent doses indicated a dose accumulation of TRX1 in the 20 mg/kg and 40 mg/kg treated animals with mean trough level concentrations increasing after each dose. Minimum TRX1 serum concentrations occurred between the first and second dose of antibody and ranged from a mean of 39.4±18.0 μg/ml (n=6) for 20 mg/kg TRX1 treated animals up to a mean of 162±63.3 μg/ml (n=6) for those receiving 40 mg/kg of TRX1. There was no dose accumulation of TRX1 in animals receiving 1 mg/kg or 10 mg/kg TRX1 as trough level concentrations determined immediately prior to the last three doses of antibody were below the limit of detection of the assay (0.2 ng/ml) as were those in control group I animals, i.e., those receiving antigen only. A protocol deviation at the time of the second TRX1 infusion eliminated one animal (#16250) from further study in the 20 mg/kg TRX1 only control group II.

TRX1 was detected by flow cytometry on CD3⁺ lymphocytes using biotinylated F(ab′)₂ donkey anti-human IgG. Twenty four hours after the first infusion MCF values were well above baseline values and remained so throughout the treatment period beginning a return to baseline levels at day 27. TRX1 was undetectable on cells by day 48. To determine the level of CD4 saturation by TRX1, biotinylated TRX1 was added to whole blood samples and cell staining assessed by flow cytometry (FIG. 10B). As expected from the TRX1 serum concentration data, free CD4 sites were readily detected in the 1 mg/kg TRX1 group. Except for the initial 24-hr time point, MCF values determined for samples obtained just prior to TRX1 dosing on days 3, 8 and 12 in the 1 mg/kg group, were only slightly below baseline values averaging 89.5% of baseline (range, 86.0%-92.9%), or 10.5% saturated. Free binding sites were also detected in the 10 mg/kg TRX1 group from samples taken just before TRX1 dosing on days 3, 8 and 12 with an average MCF value of 25.8% of baseline (range, 19.3%-33.4%) during the induction phase, indicating 74.2% of the sites were saturated. The 20 mg/kg group averaged 14.9% of baseline MCF staining (range, 10.2%-18.2%), or 85.1% saturated, during the induction phase whereas the 40 mg/kg group averaged MCF values of 9.5% of baseline (range 8.1%-10.7%), or 90.5% saturated. By day 20, one week after the last dose of TRX1, MCF values for the both 1 mg/kg and 10 mg/kg TRX1 groups had returned to baseline, while staining from the 20 mg/kg TRX1 group indicated the number of free CD4 sites at approximately 25% of baseline. The 40 mg/kg TRX1 group maintained maximum saturation at day 20, but free CD4 sites were detected on day 27 with average MCF values at 24.7% of baseline, reflecting 75.3% saturation. By day 48 MCF values had returned to baseline for both the 20 mg/kg and 40 mg/kg TRX1 groups. Reappearance of free CD4 sites correlated with the reduction in TRX1 serum concentrations during the washout phase with biotinylated TRX1 staining first beginning to increase once TRX1 serum levels dropped below approximately 10 μg/ml.

One animal in the 20 mg/kg TRX1 experimental group (#15983) showed a more rapid return to baseline of free CD4 sites as well as a more rapid clearance of TRX1 from the serum. That this was due to the development of an immune response against TRX1 was subsequently confirmed by ELISA. Of note this animal had the lowest TRX1 serum concentration trough level of all animals in the 20 mg/kg TRX1 group, 13.4 μg/ml on day 4, between the first and second dose of antibody. All other animals in the group had TRX1 serum concentrations≧35.0 μg/ml. Data from this animal are not included in the 20 mg/kg group mean calculations. All animals in the 1 mg/kg (3/3) and 10 mg/kg (3/3) TRX1 experimental groups made an immune response to TRX1 detectable by ELISA 7-10 days after the first dose of antibody. Only one other animal (#16313) made a detectable immune response to TRX1, this occurring in the 40 mg/kg TRX1 control group II. However, this response was not detectable until day 27, more than 2 weeks after the last dose of TRX1.

No treatment-related adverse events were observed during infusions or at any time after TRX1 dosing for the duration of the study. Complete blood counts and flow cytometry data showed no apparent depletion of CD4⁺ lymphocytes at any dose. While day-to-day variability in lymphocyte counts were evident, no significant differences between TRX1 treated animals and those receiving saline were observed, nor were any dose dependent differences evident among the TRX1 treated animals (FIG. 10C). Similar to the in vitro assessment, only modest CD4 modulation from the cell surface was observed.

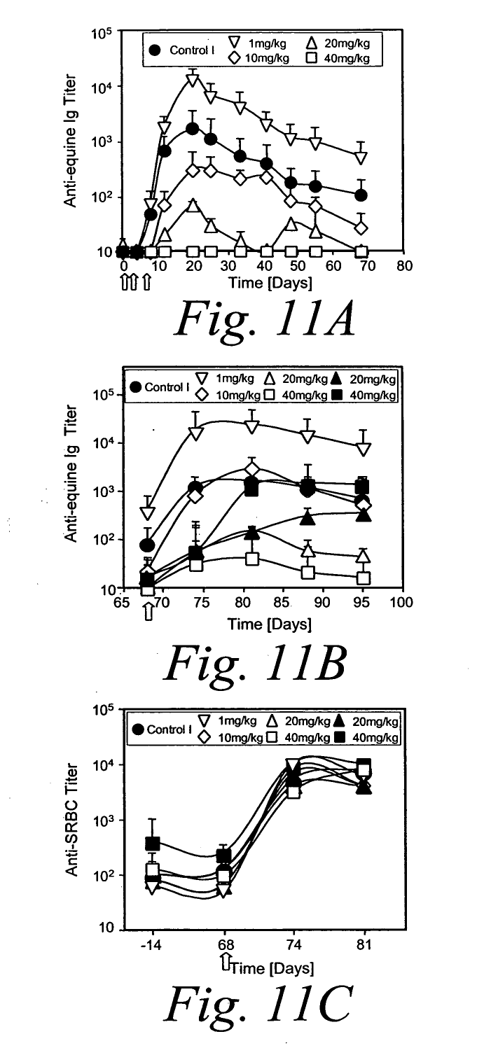

Administration of TRX1 did result in a dose dependent inhibition of the humoral response to equine Ig during the induction and washout phases (FIG. 11A). No immune response to equine Ig was detected in any animals in the 40 mg/kg TRX1 experimental group throughout this period. However, an elevation in the group mean titers against equine Ig was evident for the 20 mg/kg TRX1 experimental group. Two of 3 animals in this group, #16276 and #16096, responded with maximum peak titers of less than 10-fold above baseline, this occurring on day 27 followed by a return to baseline by day 48. Animal #15983, the same animal in which an immune response to TRX1 was observed, mounted a larger and more sustained response to equine Ig during the induction and washout phases peaking on day 41 at >25-fold above baseline and remaining >10-fold above baseline through the washout phase. Higher titers were also evident in both the 1 mg/kg and 10 mg/kg TRX1 experimental groups as well as in control group I (antigen only). Surprisingly, mean titers for the 1 mg/kg TRX1 experimental group were approximately 10- to 15-fold above those for control group I. One explanation for this apparently enhanced response may be priming to human Ig epitopes cross-reactive with equine Ig.

### Example 7

### TRX1 Induces Antigen Specific Hyporesponsiveness and Tolerance

Once TRX1 serum levels fell below the limit of detection, tolerance to equine Ig was assessed by challenging animals with immunogenic, heat-aggregated antigen and measuring the resulting specific humoral immune response. Animals were first challenged by s.c. administration of 10 mg/kg of equine Ig on day 68. All animals in the 1 mg/kg and 10 mg/kg TRX1 dose groups generated a robust secondary immune response equine Ig with group mean antibody titers closely matching that of control group I (FIG. 11B). The response was characterized by a rapid rise in antibody titer as well as higher maximum titers compared with the response observed in these groups during the tolerization phase. Showing no evidence of tolerance to equine Ig, animals from the 1 mg/kg and 10 mg/kg TRX1 experimental groups were released from study after the first challenge. Control group II, receiving antigen for the first time on day 68, responded with a group mean antibody titer to equine Ig rising more slowly than the recall response in control group I (FIG. 11B), as would be expected of a primary response. Group mean titers for the 20 mg/kg the 40 mg/kg TRX1 experimental groups also increased in response to challenge but with significantly reduced (50- to 250-fold) peak titers compared to control group I (FIG. 11B). One of three animals in the 20 mg/kg TRX1 experimental group responded to challenge with a rise in titer similar to control group I, this occurring in animal #15983, which had also generated an immune response to TRX1 during the induction and washout phases. The two other animals in this group, #16276 and #16096, were hyporesponsive to challenge with a maximum mean peak response 10-fold less than control group I. In the 40 mg/kg TRX1 experimental group one animal, #16192, was similarly hyporesponsive to challenge with the two other animals in this group, #16178 and # 16286, showing no response to challenge.

To demonstrate that the failure to mount a vigorous immune response upon antigen challenge in the 20 mg/kg and 40 mg/kg TRX1 experimental groups was antigen specific and not the consequence of treatment related immune suppression, immunocompetence was assessed by immunizing all animals with a third-party-antigen, SRBC, at the time of first challenge on day 68. All groups mounted an essentially equivalent anti-SRBC hemolytic response to this challenge (FIG. 11C), which was confirmed to be predominately IgG by ELISA.

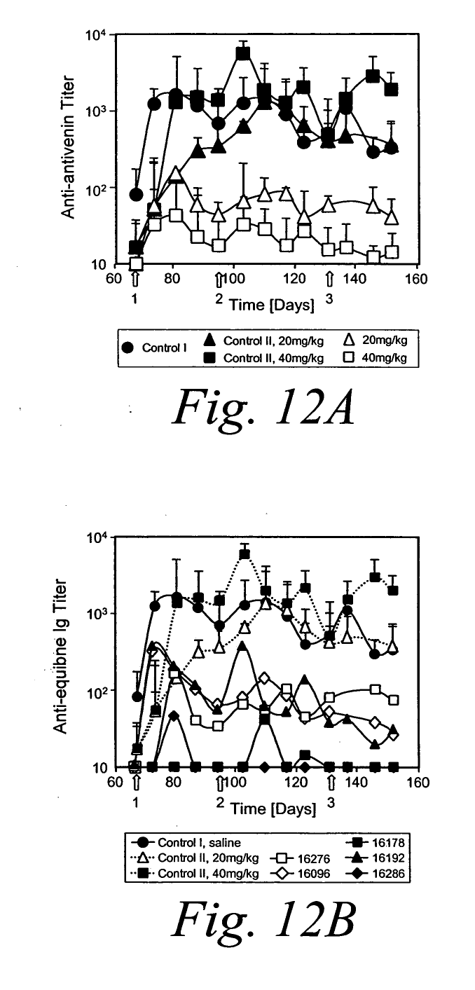

Control groups I and II as well as the 20 mg/kg and 40 mg/kg TRX1 experimental groups were re-challenged with 10 mg/kg equine Ig on day 95 and again on day 130 with 1 mg/kg equine Ig (FIG. 12A). All control groups, antigen only and TRX1 only, showed a similar boost in the humoral response to equine Ig further demonstrating that TRX1 treatment alone did not induce long-standing immune suppression. However, group mean titers for the 20 mg/kg and 40 mg/kg experimental groups failed to rise above the maximum peak titers of the first challenge even with repeated challenges. For animals in the 20 mg/kg TRX1 experimental group, excluding animal #15983, maximum titers occurred after the first challenge with peak titers of 269 and 145 for animals #16096 and #16276, respectively. Peak responses then diminished upon repeated challenge to 35 and 92, respectively, after the third challenge (FIG. 12A). Group mean titers in the 40 mg/kg TRX1 experimental group were consistently lower than those of the 20 mg/kg group with a single animal, #16192, accounting for essentially all of the response with a maximum peak titer of 313 after the first challenge. As with the animals in 20 mg/kg TRX1 group the peak response to each subsequent challenge was lower than for the previous challenge with animal #16192 response declining to a peak titer of only 39 after the third challenge with antigen (FIG. 12B). The two other animals in the 40 mg/kg TRX1 experimental group, #16178 and #16286, generated virtually no detectable immune response to equine Ig upon repeated challenge (FIG. 12B).

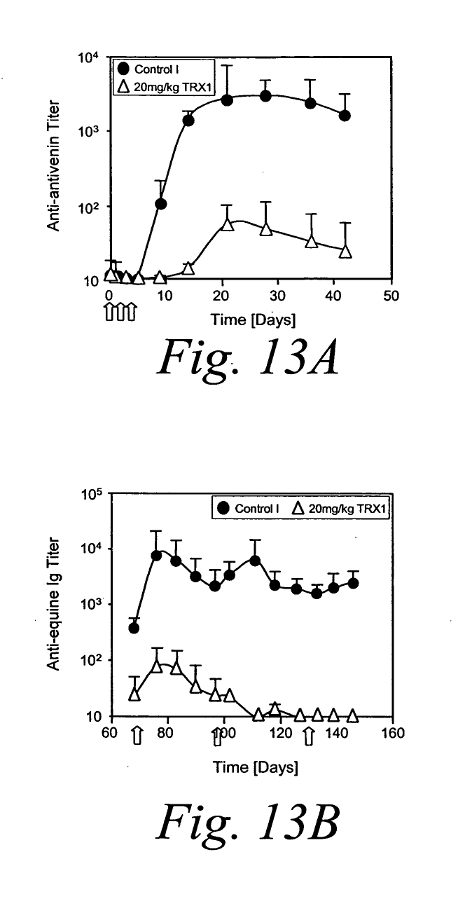

A second study (3 animals/group) was performed with the 20 mg/kg TRX1 dose reducing the number of TRX1 doses from 4 to 3 but administering them every other day on days −1, 1 and 3. A control group (control group I) was also included with animals receiving saline infusions in place of TRX1. Equine Ig dosing was unchanged with the animals receiving 3 doses of 10 mg/kg on days 0, 3 and 8. As in the first study, TRX1 administration resulted in a suppression of the humoral response to equine Ig during induction and washout phases compared to control group I with one animal, #16224, accounting for essentially all of the detectable response (FIG. 13A). On day 68 with serum levels of TRX1 below detectable levels animals were challenged with equine Ig Control group I animals responded as expected with a rapid and robust rise in titer to a mean peak response of 7652. In the 20 mg/kg TRX1 treated group, animal #16224 showed a rapid rise in titer similar to control group animals with a maximum peak titer of 6139. However, two other animals in the group, #12093 and #16130, were hyporesponsive to challenge generating peak titers of 37 and 161, respectively. A second challenge on day 97 produced only a slight rise in titer to 20 and 26 for animals #12093 and #16130, respectively, which fell rapidly to baseline. These two animals showed no response to a third challenge with antigen. As in the previous study all animals responded to SRBC neoantigen immunization at the time of first challenge on day 68.

By increasing the TRX1 dose to 20 mg/kg hyporesponsiveness was induced in two of three animals with the maximum response titer diminishing after each subsequent challenge. At doses of 40 mg/kg two of three animals were completely non-responsivene to multiple antigen challenges and the third hyporesponsive to antigen with peak response titers again declining with each antigen-challenge. Studies in mice have demonstrated that 3 doses of 20-25 mg/kg of a non-depleting anti-CD4 antibody administered every other day were sufficient to induce tolerance, although the time required for tolerance to become evident was approximately 1 month after dose initiation. The 20 mg/kg TRX1 dosing was modified, administering 3 doses, one every other day, beginning one day before antigen administration. With this modification two of three animals became completely unresponsive to antigen challenge after an initial period of hyporesponsiveness.

In man reduced immunogenicity and improved pharmacokinetics may support a lower efficacious dose of TRX1. For example, while an immune response against TRX1 has been detected in all baboons receiving only a single dose of the antibody (n=9), no immune response to TRX1 after a single dose of the antibody has been detected in man (n=9). Furthermore, a 2.5 fold increase in the serum half-life of TRX1 in man should allow for sustained CD4 coverage with less antibody compared to baboon.

No acute adverse events were associated with any dose of TRX1, and those dosing regimens that resulted in hyporesponsiveness and tolerance, while clearly immunosuppressive during the induction phase, were not associated with any clinical or histopathologic side effects. TRX1 treated animals were not housed in isolation or in germ free or specific pathogen free conditions. Despite virtually complete saturation of CD4 sites on peripheral lymphocytes for at least 21 days, no evidence for increased prevalence of enteric parasites or opportunistic bacterial, fungal, or viral infections or recrudescence of endogenous virus was found during TRX1 treatment or at any time thereafter.

It is noted that the failure of TRX1 to induce self-tolerance in the control group II animal #16313 may be due to acute infection during the tolerance induction phase with SA8 virus, an alphaherpesvirus prevalent in the baboon colony from which all animals in the study were obtained. Animal #16313 became seropositve to SA8 during the induction phase, while all other animals were either seropositive before the study, or remained seronegative throughout the study.

### Example 8

### Anti-CD4 Antibody has Effects on Monocytes/Macrophages

In this example human peripheral blood monocytes were incubated with nothing, anti-CD4 (TRX1) human IgG or aglycosyl CD8 antibody for 3, 4, or 5 days. RNA was analyzed by qualitative PCR for the levels of FcγRIIa and FγgRIIB message. TRX1 incubation was found to increase the level of FcγRIIa and FcγRIIb message.

TRX1-treated human monocyte/macrophages were also stained for CD14, CD83, CD16, CD32, CD80, CD86, MHCII, CD11b, CD62L, CCR2, and CXCR4. The phenotype of the treated cells was determined to be CD14 dim with reduced expression of CD86, CD11b, CCR2 and CXCR4. The cells had increased expression of CC16 (FcγRIII), CD32 (FcγRII) and MHC class II. The maximum effect on expression of FcγRIIb (which is known to inhibit inflammatory signals delivered by FcγRIIa and FcγRIII) was observed after 4 to 7 days.

CD4 treated murine monocyte/macrophages also made lower amounts of inflammatory cytokines (e.g., IL4, IFNγ, GM-CSF) and more cytokines associated with development of Treg cells (e.g., IL-10 and TGFβ).

Human monocytes were prepared from whole blood by isolating PBMC on Ficoll and then separating the monocytes from the lymphocytes using a negative selection kit composed of magnetic beads coupled with antibodies recognizing all cells types but monocytes and then removal of the tagged cells on a MACS cell sorting machine. Purified monocytes were incubated with nothing, human IgG (100 ug/ml or 50 ug/ml), TRX1 (50 ug/ml or 10 ug/ml) or aglycosyl anti-human CD8 antibody (50 ug/ml or 10 ug/ml) for 5 days. At that point, the cells were washed 3 times in fresh medium to remove any residual antibody and plated with allogenic purified human T cells (also purified using magnetic beads in a negative selection process) at a ratio of 2 T cells: 1 monocyte. After 5 days the cultures were fed with medium containing ³H-thymidine to measure dividing cells and cultures were harvested 18 hrs later. Data were expressed as the percent of thymidine incorporated in wells containing untreated monocytes and T cells. While anti-CD8 antibody had no effect on the MLR response (or slightly increased the response) anti-TRX1 at both 50 and 10 ug/ml reduced the response to levels below 20% of control. The human IgG at 50 ug/ml response was about 90% of control.

Numerous modifications and variations of the invention are possible in light of the above teachings; therefore, within the scope of the appended claims, the invention may be practiced otherwise than as particularly described.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

What is claimed is:

1. A method for treating a primate to induce tolerance to at least one foreign antigen comprising, administering to the primate at least one anti-CD4 antibody or CD4 binding fragment thereof, wherein the at least one anti-CD4 antibody is administered at a dose of between about 20 mg/kg to 40 mg/kg in at least three separate doses.

2. The method of claim 1, wherein the at least one anti-CD4 antibody is administered at a dose of about 20 mg/kg.

3. The method of claim 1, wherein the at least one anti-CD4 antibody is administered in at least four separate doses.

4. The method of claim 1, wherein the at least one anti-CD4 antibody is administered in at least five separate doses.

5. The method of claim 1, wherein the at least one anti-CD4 antibody is administered on at least days −1, 3 or 4, 8 and 12 relative to administration of the foreign antigen.

6. The method of claim 1, wherein the at least one anti-CD4 antibody is administered on at least days −1, 1, and 3 relative to administration of the foreign antigen.

7. The method of claim 1, wherein the foreign antigen is a soluble antigen.

8. The method of claim 1, wherein the first dose of the at least one anti-CD4 antibody is administered prior to administration of the foreign antigen.

9. The method of claim 1, wherein the at least one anti-CD4 antibody is humanized and modified to reduce Fc receptor and complement binding.

10. A method for treating a primate to induce tolerance to at least one foreign antigen comprising, administering to the primate at least one anti-CD4 antibody or CD4 binding fragment thereof, wherein at least one dose of the at least one anti-CD4 antibody is administered at least one day prior to administration of the foreign antigen.

11. A method for treating a primate to induce tolerance to at least one foreign antigen comprising, administering to the primate at least one anti-CD4 antibody or CD4 binding fragment thereof, wherein the at least one anti-CD4 antibody is administered at a dose sufficient to maintain a serum concentration of anti-CD4 antibody at a level of about 20 μg/ml during the tolerance induction phase.

12. The method of claim 11, wherein at least one dose of the at least one anti-CD4 antibody is administered one day prior to administration of the foreign antigen.

13. The method of claim 11, wherein the at least one anti-CD4 antibody is administered at a dose of between about 20 mg/kg and 40 mg/kg.

14. A method for treating a primate to induce tolerance to at least one foreign antigen comprising, administering to the primate at least one anti-CD4 antibody or CD4 binding fragment thereof, wherein the at least one anti-CD4 antibody is administered at a dose sufficient to achieve about 85% saturation of CD4 sites on T cells in the primate during the tolerance induction phase.

15. The method of claim 14, wherein the at least one anti-CD4 antibody is not administered for more than about two weeks.

16. The method of claim 14, wherein at least one dose of the at least one anti-CD4 antibody is administered one day prior to administration of the foreign antigen.

17. The method of claim 14, wherein the at least one anti-CD4 antibody is administered at a dose of between about 20 mg/kg and 40 mg/kg.

18. The method of claim 14, wherein the at least one anti-CD4 antibody is humanized and modified to reduce Fc receptor and complement binding.

19. A method for inducing tolerance in a primate to a soluble antigen, comprising, administering to a primate at least one anti-CD4 antibody or CD4 binding fragment thereof, wherein the at least one anti-CD4 antibody is administered at a dose of between about 20-40 mg/kg in at least three separate doses, such that tolerance to the soluble antigen is induced.

20. The method of claim 19, wherein at least one dose of the at least one anti-CD4 antibody is administered one day prior to administration of the soluble antigen.

21. The method of claim 19, wherein the at least one anti-CD4 antibody is administered at a dose of about 20 mg/kg.


SEQUENCE LISTING (76 sequences)

SEQ ID NO: 1 - protein, 238 aa, Artificial Sequence
  Description of Artificial Sequence Synthetic TRX1 antibody light chain construct
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - DNA, 717 bp, Artificial Sequence
  Description of Artificial Sequence Synthetic TRX1 antibody light chain construct
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - protein, 238 aa, Artificial Sequence; identical to SEQ ID NO: 1
  Description of Artificial Sequence Synthetic TRX1 antibody light chain construct

SEQ ID NO: 4 - protein, 218 aa, Artificial Sequence
  Description of Artificial Sequence Synthetic TRX1 antibody light chain construct
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 467 aa, Artificial Sequence
  Description of Artificial Sequence Synthetic TRX1 antibody heavy chain construct
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1404 bp, Artificial Sequence
  Description of Artificial Sequence Synthetic TRX1 antibody heavy chain construct
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 7 - protein, 467 aa, Artificial Sequence; identical to SEQ ID NO: 5
  Description of Artificial Sequence Synthetic TRX1 antibody heavy chain construct

SEQ ID NO: 8 - protein, 448 aa, Artificial Sequence
  Description of Artificial Sequence Synthetic TRX1 antibody heavy chain construct
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 238 aa, Artificial Sequence
  Description of Artificial Sequence Synthetic TRX1 antibody light chain construct
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 717 bp, Artificial Sequence
  Description of Artificial Sequence Synthetic TRX1 antibody light chain construct
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcactatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - protein, 238 aa, Artificial Sequence; identical to SEQ ID NO: 9
  Description of Artificial Sequence Synthetic TRX1 antibody light chain construct

SEQ ID NO: 12 - protein, 218 aa, Artificial Sequence
  Description of Artificial Sequence Synthetic TRX1 antibody light chain construct
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 467 aa, Artificial Sequence
  Description of Artificial Sequence Synthetic TRX1 antibody heavy chain construct
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1404 bp, Artificial Sequence
  Description of Artificial Sequence Synthetic TRX1 antibody heavy chain construct
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 467 aa, Artificial Sequence; identical to SEQ ID NO: 13
  Description of Artificial Sequence Synthetic TRX1 antibody heavy chain construct

SEQ ID NO: 16 - protein, 448 aa, Artificial Sequence
  Description of Artificial Sequence Synthetic TRX1 antibody heavy chain construct
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 238 aa, Artificial Sequence; identical to SEQ ID NO: 9
  Description of Artificial Sequence Synthetic TRX1 antibody light chain sequence

SEQ ID NO: 18 - DNA, 717 bp, Artificial Sequence; identical to SEQ ID NO: 10
  Description of Artificial Sequence Synthetic TRX1 antibody light chain sequence

SEQ ID NO: 19 - protein, 238 aa, Artificial Sequence; identical to SEQ ID NO: 9
  Description of Artificial Sequence Synthetic TRX1 antibody light chain construct

SEQ ID NO: 20 - protein, 218 aa, Artificial Sequence; identical to SEQ ID NO: 12
  Description of Artificial Sequence Synthetic TRX1 antibody light chain construct

SEQ ID NO: 21 - protein, 467 aa, Artificial Sequence; identical to SEQ ID NO: 5
  Description of Artificial Sequence Synthetic TRX1 heavy chain construct

SEQ ID NO: 22 - DNA, 1404 bp, Artificial Sequence; identical to SEQ ID NO: 6
  Description of Artificial Sequence Synthetic TRX1 heavy chain construct

SEQ ID NO: 23 - protein, 467 aa, Artificial Sequence; identical to SEQ ID NO: 5
  Description of Artificial Sequence Synthetic TRX1 heavy chain construct

SEQ ID NO: 24 - protein, 448 aa, Artificial Sequence; identical to SEQ ID NO: 8
  Description of Artificial Sequence Synthetic TRX1 heavy chain construct

SEQ ID NO: 25 - protein, 238 aa, Artificial Sequence; identical to SEQ ID NO: 1
  Description of Artificial Sequence Synthetic TRX1 antibody light chain construct

SEQ ID NO: 26 - DNA, 717 bp, Artificial Sequence; identical to SEQ ID NO: 2
  Description of Artificial Sequence Synthetic TRX1 antibody light chain construct

SEQ ID NO: 27 - protein, 238 aa, Artificial Sequence; identical to SEQ ID NO: 1
  Description of Artificial Sequence Synthetic TRX1 light chain construct

SEQ ID NO: 28 - protein, 218 aa, Artificial Sequence; identical to SEQ ID NO: 4
  Description of Artificial Sequence Synthetic TRX1 light chain construct

SEQ ID NO: 29 - protein, 467 aa, Artificial Sequence; identical to SEQ ID NO: 13
  Description of Artificial Sequence Synthetic TRX1 heavy chain construct

SEQ ID NO: 30 - DNA, 1404 bp, Artificial Sequence; identical to SEQ ID NO: 14
  Description of Artificial Sequence Synthetic TRX1 heavy chain construct

SEQ ID NO: 31 - protein, 467 aa, Artificial Sequence; identical to SEQ ID NO: 13
  Description of Artificial Sequence Synthetic TRX1 heavy chain construct

SEQ ID NO: 32 - protein, 448 aa, Artificial Sequence; identical to SEQ ID NO: 16
  Description of Artificial Sequence Synthetic TRX1 heavy chain construct

SEQ ID NO: 33 - protein, 451 aa, Artificial Sequence
  Description of Artificial Sequence Humanized CD8 antibody heavy chain construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, Artificial Sequence
  Description of Artificial Sequence Humanized CD8 antibody light chain construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 135 aa, Homo sapiens
MVLQTQVFISLLLWISGAYGDIVMTQSPDSLAVSLGERATINCKSSQSLLYSSNNKNYLAWYQQKPGQPPKLLIYWASTRESGVPDRFSGSGSGTDFTLTISSLQAEDVAVYYCQQYYSTPPMFGQGTKVEIKRT

SEQ ID NO: 36 - protein, 142 aa, Homo sapiens
LLAVAPGAHSQVQLVQSGAEVKKPGASVKVSCKASGYTFTNYYMHWVRQAPGQGLEWMGIINPSGNSTNYAQKFQGRVTMTRDTSTSTVYMELSSLRSEDTAVYYCAREKLATTIFGVLIITGMDYWGQGTLVTVSSGSASA

SEQ ID NO: 37 - DNA, 76 bp, Artificial Sequence
  Description of Artificial Sequence Synthetic primer
tgacattgtgatgacccaatctccagattctttggctgtgtctctaggtgagagggccaccatcaactgcaaggcc

SEQ ID NO: 38 - DNA, 29 bp, Artificial Sequence
  Description of Artificial Sequence Synthetic primer
tgaactggtatcaacagaaaccaggacag

SEQ ID NO: 39 - DNA, 28 bp, Artificial Sequence
  Description of Artificial Sequence Synthetic primer
agagtctggggtcccagacaggtttagt

SEQ ID NO: 40 - DNA, 42 bp, Artificial Sequence
  Description of Artificial Sequence Synthetic primer
gtcttcaggaccctccgacgttcggtggaggtaccaagctgg

SEQ ID NO: 41 - DNA, 52 bp, Artificial Sequence
  Description of Artificial Sequence Synthetic primer
caccctcaccatcagttctctgcaggcggaggatgttgcagtctattagtgt

SEQ ID NO: 42 - DNA, 24 bp, Artificial Sequence
  Description of Artificial Sequence Synthetic primer
agctttacagttactgagcacaca

SEQ ID NO: 43 - DNA, 24 bp, Artificial Sequence
  Description of Artificial Sequence Synthetic primer
tcgatgtgtgctcagtaactgtaa

SEQ ID NO: 44 - DNA, 75 bp, Artificial Sequence
  Description of Artificial Sequence Synthetic primer
ggttcagctggtgcagtctggagctgaagtgaagaagcctggggcttcagtgaaggtgtcctgtaaggcttctgg

SEQ ID NO: 45 - DNA, 52 bp, Artificial Sequence
  Description of Artificial Sequence Synthetic primer
agctgggtgaggcaggcacctggacagggccttgagtggatgggagagattt

SEQ ID NO: 46 - DNA, 60 bp, Artificial Sequence
  Description of Artificial Sequence Synthetic primer
caagggcagggtcacaatgactagagacacatccaccagcacagtctacatggaactcag

SEQ ID NO: 47 - DNA, 43 bp, Artificial Sequence
  Description of Artificial Sequence Synthetic primer
cagcctgaggtctgaggacactgcggtctattactgtgcaaga

SEQ ID NO: 48 - DNA, 24 bp, Artificial Sequence
  Description of Artificial Sequence Synthetic primer
gccaagggacactagtcactgtgt

SEQ ID NO: 49 - DNA, 39 bp, Artificial Sequence
  Description of Artificial Sequence Synthetic primer
actctaaccatggaatggatctggatctttctcctcatc

SEQ ID NO: 50 - DNA, 39 bp, Artificial Sequence
  Description of Artificial Sequence Synthetic primer
tcactgcctatgttataagctgggtgaggcaggcacctg

SEQ ID NO: 51 - DNA, 21 bp, Artificial Sequence
  Description of Artificial Sequence Synthetic primer
actagtcacagtctcctcagc

SEQ ID NO: 52 - DNA, 22 bp, Artificial Sequence
  Description of Artificial Sequence Synthetic primer
gaattcatttacccggagacag

SEQ ID NO: 53 - DNA, 49 bp, Artificial Sequence
  Description of Artificial Sequence Synthetic primer
ccgtgcccagcacctgaactcgcgggggcaccgtcagtcttcctccccc

SEQ ID NO: 54 - DNA, 26 bp, Artificial Sequence
  Description of Artificial Sequence Synthetic primer
ggtaccaaggtggaaatcaaacgaac

SEQ ID NO: 55 - DNA, 25 bp, Artificial Sequence
  Description of Artificial Sequence Synthetic primer
aagcttctaacactctcccctgttg

SEQ ID NO: 56 - DNA, 78 bp, Artificial Sequence
  Description of Artificial Sequence Synthetic primer
aagcttatggaatggatctggatctttctcctcatcctgtcaggaactcgaggtgtccagtcccaggttcagctggtg

SEQ ID NO: 57 - DNA, 74 bp, Artificial Sequence
  Description of Artificial Sequence Synthetic primer
ctgtaaggcttctggatacacattcactgcctatgttataagctgggtgaggcaggcacctggacagggccttg

SEQ ID NO: 58 - DNA, 70 bp, Artificial Sequence
  Description of Artificial Sequence Synthetic primer
ggtagtagttattataatgagaagttcaagggcagggtcacaatgactagagacacatccaccagcacag

SEQ ID NO: 59 - DNA, 77 bp, Artificial Sequence
  Description of Artificial Sequence Synthetic primer
gaggacactgcggtctattactgtgcaagatccggggacggcagtcggtttgtttactggggccaagggacactagt

SEQ ID NO: 60 - DNA, 90 bp, Artificial Sequence
  Description of Artificial Sequence Synthetic primer
gtgtatccagaagccttacaggacaccttcactgaagccccaggcttcttcacttcagctccagactgcaccagctgaacctgggactgg

SEQ ID NO: 61 - DNA, 77 bp, Artificial Sequence
  Description of Artificial Sequence Synthetic primer
cttctcattataataactactaccgcttccaggataaatctctcccatccactcaaggccctgtccaggtgcctgcc

SEQ ID NO: 62 - DNA, 71 bp, Artificial Sequence
  Description of Artificial Sequence Synthetic primer
gtaatagaccgcagtgtcctcagacctcaggctgctgagttccatgtagactgtgctggtggatgtgtctc

SEQ ID NO: 63 - DNA, 69 bp, Artificial Sequence
  Description of Artificial Sequence Synthetic primer
gaattcatggagacagacacaatcctgctatgggtgctgctgctctgggttccaggctccactggtgac

SEQ ID NO: 64 - DNA, 66 bp, Artificial Sequence
  Description of Artificial Sequence Synthetic primer
ggctgtgtctctaggtgagagggccaccatcaactgcaaggccagccaaagtgttgattatgatgg

SEQ ID NO: 65 - DNA, 65 bp, Artificial Sequence
  Description of Artificial Sequence Synthetic primer
cagaaaccaggacagccacccaaactcctcatctatgttgcatccaatctagagtctggggtccc

SEQ ID NO: 66 - DNA, 66 bp, Artificial Sequence
  Description of Artificial Sequence Synthetic primer
ggacagacttcaccctcaccatcagttctctgcaggcggaggatgttgcagtctattactgtcagc

SEQ ID NO: 67 - DNA, 67 bp, Artificial Sequence
  Description of Artificial Sequence Synthetic primer
cacctagagacacagccaaagaatctggagattgggtcatcacaatgtcaccagtggagcctggaac

SEQ ID NO: 68 - DNA, 66 bp, Artificial Sequence
  Description of Artificial Sequence Synthetic primer
ggtggctgtcctggtttctgttgataccagttcatataactatcaccatcataatcaacactttgg

SEQ ID NO: 69 - DNA, 67 bp, Artificial Sequence
  Description of Artificial Sequence Synthetic primer
ggtgagggtgaagtctgtcccagacccactgccactaaacctgtctgggaccccagactctagattg

SEQ ID NO: 70 - DNA, 60 bp, Artificial Sequence
  Description of Artificial Sequence Synthetic primer
ggtacctccaccgaacgtcggagggtcctgaagactttgctgacagtaatagactgcaac

SEQ ID NO: 71 - DNA, 1404 bp, Artificial Sequence
  Description of Artificial Sequence Synthetic TRX1 antibody heavy chain construct
atggaatggatctggatctttctcctcatcctgtcaggaactgcaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 72 - protein, 467 aa, Artificial Sequence
  Description of Artificial Sequence Synthetic TRX1 antibody heavy chain construct
MEWIWIFLLILSGTAGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 717 bp, Artificial Sequence; identical to SEQ ID NO: 10
  Description of Artificial Sequence Synthetic TRX1 antibody light chain construct

SEQ ID NO: 74 - protein, 238 aa, Artificial Sequence; identical to SEQ ID NO: 9
  Description of Artificial Sequence Synthetic TRX1 antibody light chain construct

SEQ ID NO: 75 - DNA, 1356 bp, Artificial Sequence
  Description of Artificial Sequence Humanized CD8 antibody heavy chain construct
caggttcaattggtggagtctggaggaggcgttgtacagcctggaaggtccctgagactctcatgtgcagcttctggattcactttcagtgactttggcatgaactgggttcgacaggctcccgggaaggggctggaatgggtggcactgatttactatgatggtagtaacaagttctatgcagactctgtgaagggtcgattcaccatctccagggacaattctaagaacaccctatacctgcaaatgaacagcctgagagctgaggacacagccgtgtattactgtgcaaaaccccactatgatggttattatcacttctttgattcctggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 76 - DNA, 642 bp, Artificial Sequence
  Description of Artificial Sequence Humanized CD8 antibody light chain construct
gacatccagatgacccagagcccaagcagcctgagcgccagcgtgggtgacagagtgaccatcacctgtaaaggaagtcaggatattaacaattacttagcctggtaccagcagaagccaggtaaggctccaaagctgctgatctacaatacagacattttgcacacgggtgtgccaagcagattcagcggtagcggtagcggtaccgacttcaccttcaccatcagcagcctccagccagaggacatcgccacctactactgctatcagtataacaacgggtacacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag